(19) United States
(12) Patent Application Publication
(10) Pub. No.: US 2010/0190764 A1
(43) Pub. Date: Jul. 29, 2010

(54) Novel compounds

(75) Inventors: Emiliano Castiglioni, Verona (IT); Romano Di Fabio, Verona (IT); Massimo Gianotti, Verona (IT); Milan Mesic, Zagreb (HR); Francesca Pavone, Verona (IT); Slavko Rast, Zagreb (HR); Luigi Piero Stasi, Verona (IT)

Correspondence Address: GlaxoSmithKline;GLOBAL PATENTS -US, UW2220, P. O. BOX 1539, KING OF PRUSSIA, PA, 19406-0939, US

(73) Assignee: Glaxo Group Limited

(21) Appl. No.: 12/670,712

(22) PCT Filed: Jul. 24, 2008
(86) PCT No.: PCT/EP2008/059682

§ 371 Date: Jan. 26, 2010

(30) Foreign Application Priority Data
Jul. 27, 2007 (GB) 0714712.7
Nov. 28, 2007 (GB) 0723318.2
Apr. 7, 2008 (GB) 0806289.5

(51) Int. Cl. C07C 211/42; A61K 31/397; A61K 31/5375; A61K 31/495; A61K 31/44; A61K 31/403; A61K 31/195; A61K 31/135; C07D 265/30; C07D 241/04; C07D 213/04; C07D 209/54; C07D 205/04; C07C 229/34; A61P 25/20

(52) U.S. Cl. 514/210.17; 514/237.5; 514/255.01; 514/278; 514/409; 514/567; 514/647; 544/70; 544/230; 546/15; 548/407; 548/953; 562/457; 564/308

(57) ABSTRACT

This invention relates to novel Spiro cyclopentane derivatives of formula (I) or a pharmaceutically acceptable salt thereof, for treating diseases and conditions of the central nervous system (CNS), in particular sleep disorders.

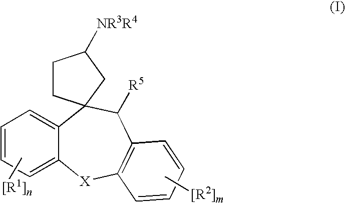

This invention relates to novel spiro cyclopentane derivatives. The invention also relates to the use of the derivatives in treating diseases and conditions of the central nervous system (CNS), in particular sleep disorders. In addition, the invention relates to compositions containing the derivatives and processes for their preparation.

Common symptoms of those suffering with a sleep disorder include abnormal sleep behaviour and difficulties in one or more of falling asleep, remaining asleep, sleeping for adequate lengths of time and achievement of restorative sleep.

Available treatments for sleep disorders include the use of prescription hypnotics, e.g., benzodiazepines. However, these may be habit-forming, lose their effectiveness after extended use, and metabolise more slowly for certain designated groups, resulting in persisting medicative effects.

Other treatments include over-the-counter antihistamines, e.g., diphenhydramine and dimenhydrinate. These are not designed to be strictly sedative in their activity and as such, this method of treatment has been associated with a number of adverse side effects, e.g., persistence of the sedating medication after the prescribed time of treatment, or the so-called “hangover effect”. Many of these side effects result from non-specific activity in both the periphery as well as the CNS during this period of extended medication.

It has been suggested that brain histamine is involved in the regulation of the sleep-wake cycle, arousal, cognition and memory mainly through H₁ receptors, producing a reduction of the sleep latency in both preclinical (Shigemoto et al., (2004), Eur J Pharmacol., 494(2-3):161-5) and clinical studies (Simons et al., (1996), Clin Exp Allergy, 26(9):1092-7).

In parallel, selective blockade of the 5-HT_{2A} receptor has been proved in both preclinical studies (Popa et al., (2005), J. Nuerosc., 25(49): 11231-8) and clinical studies (Viola A. et al, (2002), Clin. Neurophysiol., 113(3) 429-434) to be efficacious in reducing Wake After Sleep Onset, increasing Slow Wave Sleep and Total Sleep Time therefore providing consolidation of sleep.

Therefore, a need exists for the development of improved drug therapies useful for the treatment of sleep disorders.

In a first aspect, the invention provides a compound of formula (I), or a pharmaceutically acceptable salt thereof

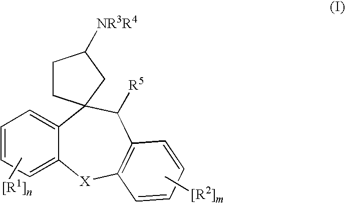

wherein

X is CH₂, C═O, O, or S;

n is 0, 1 or 2;

m is 0, 1 or 2;

when present, R¹ is independently selected from the list consisting of C₁₋₄alkyl, C₁₋₄alkoxy and halogen;

when present, R² is independently selected from the list consisting of C₁₋₄alkyl, C₁₋₄alkoxy and halogen;

R³ and R⁴ are independently selected from the list consisting of hydrogen, C₁₋₆alkyl, carboxy and carboxyC₁₋₆alkyl; or

R³ and R⁴, together with the nitrogen to which they are attached, form a 4-7 membered saturated or partially unsaturated ring optionally containing one or more additional heteroatoms independently selected from N, S and O, the ring being optionally substituted by one or more groups independently selected from halogen, C₁₋₃alkoxycarbonyl, carboxy, C₁₋₆alkyl, carboxyC₁₋₆alkyl, hydroxy and —C(O)NR^{a}R^{b}; or

R³ and R⁴, together with the nitrogen to which they are attached, form a 6 membered azabicyclic ring optionally substituted by one or more groups independently selected from halogen, C₁₋₃alkoxycarbonyl, carboxy, C₁₋₆alkyl, carboxyC₁₋₆alkyl, hydroxy and —C(O)NR^{a}R^{b};

R^{a} and R^{b} are independently selected from the list consisting of hydrogen, C₁₋₃alkyl and C₁₋₃alkoxy; and

R⁵ is hydrogen or oxo.

The term “halogen” and its abbreviation “halo” refer to fluorine, chlorine, bromine or iodine. In an embodiment unless otherwise indicated such a halo substituents is fluoro or chloro.

As used herein, a C₁₋₆alkyl substituent is a univalent radical derived by removal of a hydrogen atom from an acyclic C₁₋₆alkane. Such C₁₋₆alkyl substituents include methyl and ethyl, may be straight chain (i.e. n-propyl, n-butyl, n-pentyl and n-hexyl) or branched chain (for example, isopropyl, isobutyl, secbutyl, tert-butyl, isopentyl and neopentyl). In an embodiment, unless otherwise indicated, any C₁₋₆alkyl substituent is methyl, ethyl, n-propyl or isopropyl.

As used herein, a C₁₋₄alkyl substituent is a univalent radical derived by removal of a hydrogen atom from an acyclic C₁₋₄alkane. Such C₁₋₄alkyl substituents include methyl and ethyl, may be straight chain (i.e. n-propyl, n-butyl) or branched chain (for example, isopropyl, isobuty). In an embodiment, unless otherwise indicated, any C₁₋₄alkyl substituent is methyl, ethyl, n-propyl or isopropyl.

As used herein, a C₁₋₄alkoxy substituent is group of formula “R—O-” where R is C₁₋₄alkyl as defined above. Such alkoxy substituents include methoxy and ethoxy and may be straight chain (i.e. n-propoxy and n-butoxy) or branched chain (for example, isopropoxy and isobutoxy). In an embodiment, unless otherwise indicated, any C₁₋₄alkoxy substituent is methoxy, ethoxy, n-propoxy or isopropoxy.

As used herein unless otherwise indicated, a carboxy substituent is —CO(O)—OH.

As used herein unless otherwise indicated, a carboxyC₁₋₆alkyl substituent is group of formula HO—C(O)-alkylene-. For example, HO—C(O)—CH₂—.

As used herein, unless otherwise indicated, a C₁₋₃alkoxycarbonyl substituent is R—O—C(O)—, wherein R is C₁₋₃alkyl. For example, CH₃—O—C(O)—.

As used herein, the term “oxo” is the bivalent radical ═O.

As used herein, a 4-7 membered saturated or partially unsaturated ring is a monocyclic ring which may be saturated or partially unsaturated containing at least one nitrogen atom and optionally containing from 1 to 4 additional heteroatoms selected from oxygen, nitrogen or sulphur. In an embodiment the ring is selected from pyrrolinyl, pyrrolidinyl, azetidinyl, pyrazolidinyl, oxazolidinyl, piperidinyl, piperazinyl, morpholinyl, thiomorpholinyl, thiazolidinyl, hydantoinyl, dihydropyranyl, tetrahydropyranyl, tetrahydropyridinyl, tetrahydropyrimidinyl, diazepanyl and azepanyl.

As used herein, a 6 membered saturated or partially unsaturated ring is a monocyclic ring which may be saturated or partially unsaturated containing at least one nitrogen atom and optionally containing from 1 to 4 additional heteroatoms selected from oxygen, nitrogen or sulphur. In an embodiment the ring is selected from piperidinyl, piperazinyl, morpholinyl, thiomorpholinyl, tetrahydropyridinyl, tetrahydropyrimidinyl, and tetrahydrothiopyranyl.

As used herein, a 6 membered azabicyclic ring is a 6 membered saturated bicyclic ring containing only 1 nitrogen atom. In an embodiment the the ring is 3-azabicyclo[3.1.0]hexane.

In a second aspect, the invention provides a compound of formula (I) or a pharmaceutically acceptable salt thereof

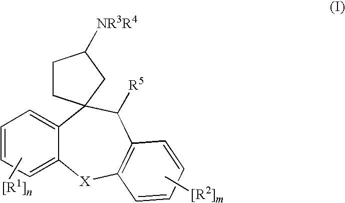

wherein

X is CH₂, C═O, O, or S;

n is 0, 1 or 2;

m is 0, 1 or 2;

when present, R¹ is independently selected from the list consisting of C₁₋₄olkyl, C₁₋₄alkoxy and halogen;

when present, R² is independently selected from the list consisting of C₁₋₄alkyl, C₁₋₄alkoxy and halogen;

R³ and R⁴ are independently selected from the list consisting of hydrogen, C₁₋₆alkyl, carboxy and carboxyC₁₋₆alkyl; or

R³ and R⁴, together with the nitrogen to which they are attached, form a 4-7 membered saturated or partially unsaturated ring optionally containing one or more additional heteroatoms independently selected from N, S and O, the ring being optionally substituted by one or more groups independently selected from halogen, C₁₋₃alkoxycarbonyl, carboxy and C₁₋₆alkyl; or

R³ and R⁴, together with the nitrogen to which they are attached, form a 6 membered azabicyclic ring optionally substituted by one or more groups independently selected from halogen, C₁₋₃alkoxycarbonyl, carboxy and C₁₋₆alkyl; and

R⁵ is selected from the list consisting of hydrogen and oxo.

In an embodiment, X is CH₂, O or S. In a further embodiment, X is CH₂ or O.

In an embodiment, n is 0 or 1. In a further embodiment, when present, R¹ is fluoro or chloro.

In an embodiment, m is 0 or 1. In a further embodiment, when present, R² is fluoro or chloro.

In an embodiment, n is 0, m is 1 and R² is halogen. In a further embodiment, n is 0, m is 1 and R² is either fluoro or chloro.

In an embodiment, R³ is hydrogen or C₁₋₄alkyl and R⁴ is carboxy or carboxyC₁₋₄alkyl.

In an embodiment, R³ and R⁴, together with the nitrogen to which they are attached, form a 5-6 membered saturated or partially unsaturated ring optionally containing one additional heteroatom selected from N, S and O, the ring being optionally substituted by one or more groups selected independently from halogen, C₁₋₃alkoxycarbonyl, carboxy and C₁₋₆alkyl. In an embodiment the ring is selected from tetrahydropyridinyl, oxazolidinyl, morpholinyl, piperazinyl, pyrrolidinyl, piperidinyl and thiomorpholinyl.

In an embodiment, R³ and R⁴, together with the nitrogen to which they are attached, form a 5-6 membered saturated or partially unsaturated ring optionally containing one additional heteroatom selected from N, S and O, the ring being optionally substituted by one or more groups selected independently from halogen and carboxy. In an embodiment the ring is selected from tetrahydropyridinyl, oxazolidinyl, morpholinyl, piperazinyl, pyrrolidinyl, piperidinyl and thiomorpholinyl.

In an embodiment, R³ and R⁴, together with the nitrogen to which they are attached, form a 5-6 membered saturated or partially unsaturated ring optionally containing one additional heteroatom selected from N, S and O, the ring being optionally substituted by carboxy. In an embodiment the ring is selected from tetrahydropyridinyl, oxazolidinyl, morpholinyl, piperazinyl, pyrrolidinyl, piperidinyl and thiomorpholinyl.

In an embodiment, R³ and R⁴, together with the nitrogen to which they are attached, form a 5-6 membered saturated or partially unsaturated ring optionally containing one additional heteroatom selected from N, S and O, the ring being substituted by one or more groups selected independently from halogen, C₁₋₃alkoxycarbonyl, carboxy and C₁₋₆alkyl. In an embodiment, the ring is selected from tetrahydropyridinyl, oxazolidinyl, morpholinyl, piperazinyl, pyrrolidinyl, piperidinyl and thiomorpholinyl.

In an embodiment, R³ and R⁴, together with the nitrogen to which they are attached, form a 5-6 membered saturated or partially unsaturated ring optionally containing one additional heteroatom selected from N, S and O, the ring being substituted by one or more groups selected independently from halogen and carboxy. In an embodiment the ring is selected from tetrahydropyridinyl, oxazolidinyl, morpholinyl, piperazinyl, pyrrolidinyl, piperidinyl and thiomorpholinyl.

In an embodiment, R³ and R⁴, together with the nitrogen to which they are attached, form a 5-6 membered saturated or partially unsaturated ring optionally containing one additional heteratom selected from N, S and O, the ring being substituted by carboxy. In an embodiment the ring is selected from tetrahydropyridinyl, oxazolidinyl, morpholinyl, piperazinyl, pyrrolidinyl, piperidinyl and thiomorpholinyl.

In a further embodiment the ring is selected from tetrahydropyridinyl, morpholinyl, piperazinyl and piperidinyl.

In a preferred embodiment, R³ and R⁴, together with the nitrogen to which they are attached, form an azetidinyl, the ring being optionally substituted by one or more groups selected independently from halogen, C₁₋₃alkoxycarbonyl, carboxy and C₁₋₆alkyl.

In a further preferred embodiment, R³ and R⁴, together with the nitrogen to which they are attached, form an azetidinyl, the ring being substituted by carboxy.

In a still further preferred embodiment, R³ and R⁴, together with the nitrogen to which they are attached, form a 3-carboxyazetidinyl.

In a further embodiment, R³ and R⁴, together with the nitrogen to which they are attached, form a 6 membered saturated or partially unsaturated ring optionally containing one additional heteroatom selected from N, S and O, the ring being optionally substituted by one or more groups selected independently from halogen, C₁₋₃alkoxycarbonyl, carboxy and C₁₋₆alkyl.

In a further embodiment, R³ and R⁴, together with the nitrogen to which they are attached, form a 6 membered saturated or partially unsaturated ring optionally containing one additional heteroatom selected from N, S and O, the ring being optionally substituted by one or more groups selected independently from halogen and carboxy.

In a further embodiment, R³ and R⁴, together with the nitrogen to which they are attached, form a 6 membered saturated or partially unsaturated ring optionally containing one additional heteroatom selected from N, S and O, the ring being optionally substituted by carboxy.

In a further embodiment, R³ and R⁴, together with the nitrogen to which they are attached, form a 6 membered saturated or partially unsaturated ring optionally containing one additional heteroatom selected from N, S and O, the ring being substituted by one or more groups selected independently from halogen, C₁₋₃alkoxycarbonyl, carboxy and C₁₋₆alkyl.

In a further embodiment, R³ and R⁴, together with the nitrogen to which they are attached, form a 6 membered saturated or partially unsaturated ring optionally containing one additional heteroatom selected from N, S and O, the ring being substituted by one or more groups selected independently from halogen and carboxy.

In a further embodiment, R³ and R⁴, together with the nitrogen to which they are attached, form a 6 membered saturated or partially unsaturated ring optionally containing one additional heteroatom selected from N, S and O, the ring being substituted by carboxy.

In a still further embodiment, R³ and R⁴, together with the nitrogen to which they are attached, form a 6 membered saturated or partially unsaturated ring containing no additional heteroatoms, the ring being optionally substituted by one or more groups selected independently from halogen, C₁₋₃alkoxycarbonyl, carboxy and C1-6alkyl.

In a still further embodiment, R³ and R⁴, together with the nitrogen to which they are attached, form a 6 membered saturated or partially unsaturated ring containing no additional heteroatoms, the ring being optionally substituted by one or more groups selected independently from halogen and carboxy.

In a still further embodiment, R³ and R⁴, together with the nitrogen to which they are attached, form a 6 membered saturated or partially unsaturated ring containing no additional heteroatoms, the ring being optionally substituted by carboxy. In a still further embodiment, R³ and R⁴, together with the nitrogen to which they are attached, form a 6 membered saturated or partially unsaturated ring containing no additional heteroatoms, the ring being substituted by one or more groups selected independently from halogen, C₁₋₃alkoxycarbonyl, carboxy and C₁₋₆alkyl.

In a still further embodiment, R³ and R⁴, together with the nitrogen to which they are attached, form a 6 membered saturated or partially unsaturated ring containing no additional heteroatoms, the ring being substituted by one or more groups selected independently from halogen and carboxy.

In a still further embodiment, R³ and R⁴, together with the nitrogen to which they are attached, form a 6 membered saturated or partially unsaturated ring containing no additional heteroatoms, the ring being substituted by carboxy.

In an embodiment, R³ and R⁴, together with the nitrogen to which they are attached, form a 6 membered azabicyclic ring optionally substituted by one or more groups independently selected from halogen, carboxy and C₁₋₆alkyl. In an embodiment, the azabicyclic ring is 3-azabicyclo[3.1.0]hexane.

In an embodiment, R³ and R⁴, together with the nitrogen to which they are attached, form a 6 membered azabicyclic ring substituted by one or more groups independently selected from halogen, carboxy and C₁₋₆alkyl. In an embodiment, the azabicyclic ring is 3-azabicyclo[3.1.0]hexane.

In an embodiment, R⁵ is hydrogen.

In an embodiment, the compound of formula (I) is selected from the list consisting of:

N-(5′,11′-Dihydrospiro[cyclopentane-1,10′-dibenzo[a,d]cyclohepten]-3-yl)-b-alanine formate salt (diastereomeric mixture 1);

N-(5′,11′-Dihydrospiro[cyclopentane-1,10′-dibenzo[a,d]cyclohepten]-3-yl)-b-alanine hydrochloride salt (diastereomeric mixture 2);

1-(5′,11′-Dihydrospiro[cyclopentane-1,10′-dibenzo[a,d]cyclohepten]-3-yl)-1,2,5,6-tetrahydro-3-pyridinecarboxylic acid hydrochloride salt (diastereomeric mixture 1);

N-(5′,11′-Dihydrospiro[cyclopentane-1,10′-dibenzo[a,d]cyclohepten]-3-yl)-N-methyl-β-alanine (diastereomeric mixture 1);

N-(5′,11′-Dihydrospiro[cyclopentane-1,10′-dibenzo[a,d]cyclohepten]-3-yl)-N-methyl-β-alanine (diastereomeric mixture 2);

4-[5′,11′-Dihydrospiro[cyclopentane-1,10′-dibenzo[a,d]cyclohepten]-3-yl(methyl)amino]butanoic acid (diastereomeric mixture 1);

1-(5′,11′-Dihydrospiro[cyclopentane-1,10′-dibenzo[a,d]cyclohepten]-3-yl)-1,2,5,6-tetrahydro-3-pyridinecarboxylic acid (isomer 2);

1-(5′,11′-Dihydrospiro[cyclopentane-1,10′-dibenzo[a,d]cyclohepten]-3-yl)-1,2,5,6-tetrahydro-3-pyridinecarboxylic acid (isomer 4);

1-(5′,11′-Dihydrospiro[cyclopentane-1,10′-dibenzo[a,d]cyclohepten]-3-yl)-4-piperidinecarboxylic acid (isomer 4);

(−) 1-(5′,11′-Dihydrospiro[cyclopentane-1,10′-dibenzo[a,d]cyclohepten]-3-yl)-4-piperidinecarboxylic acid;

1-(5′,11′-Dihydrospiro[cyclopentane-1,10′-dibenzo[a,d]cyclohepten]-3-yl)-3-piperidinecarboxylic acid (isomer 1);

1-(5′,11′-Dihydrospiro[cyclopentane-1,10′-dibenzo[a,d]cyclohepten]-3-yl)-3-piperidinecarboxylic acid (isomer 2);

1-(2′-Fluoro-11′-oxo-11′H-spiro[cyclopentane-1,10′-dibenzo[b,f]thiepin]-3-yl)-4-piperidinecarboxylic acid;

3-(5′,11′-Dihydrospiro[cyclopentane-1,10′-dibenzo[a,d]cyclohepten]-3-yl)-3-azabicyclo[3.1.0]hexane-1-carboxylic acid (isomer 1);

3-(5′,11′-Dihydrospiro[cyclopentane-1,10′-dibenzo[a,d]cyclohepten]-3-yl)-3-azabicyclo[3.1.0]hexane-1-carboxylic acid (isomer 2);

N-methyl-5′,11′-dihydrospiro[cyclopentane-1,10′-dibenzo[a,d]cyclohepten]-3-amine (diastereomeric mixture 1);

N-methyl-5′,11′-dihydrospiro[cyclopentane-1,10′-dibenzo[a,d]cyclohepten]-3-amine (diastereomeric mixture 2);

1-(5′,11′-Dihydrospiro[cyclopentane-1,10′-dibenzo[a,d]cyclohepten]-3-yl)-4-fluoro-piperidinecarboxylic acid (isomer 2);

4-(5′,11′-Dihydrospiro[cyclopentane-1,10′-dibenzo[a,d]cyclohepten]-3-yl)-2-morpholinecarboxylic acid (isomer 1);

4-(5′,11′-Dihydrospiro[cyclopentane-1,10′-dibenzo[a,d]cyclohepten]-3-yl)-2-morpholinecarboxylic acid (isomer 2);

Methyl 1-(5′,11′-dihydrospiro[cyclopentane-1,10′-dibenzo[a,d]cyclohepten]-3-yl)-1,2,5,6-tetrahydro-3-pyridinecarboxylate (diastereomeric mixture 1);

Methyl 1-(5′,11′-dihydrospiro[cyclopentane-1,10′-dibenzo[a,d]cyclohepten]-3-yl)-1,2,5,6-tetrahydro-3-pyridinecarboxylate (diastereomeric mixture 2);

Methyl 1-(5′,11′-dihydrospiro[cyclopentane-1,10′-dibenzo[a,d]cyclohepten]-3-yl)-1,2,5,6-tetrahydro-3-pyridinecarboxylate (isomer 2);

Methyl 1-(5′,11′-dihydrospiro[cyclopentane-1,10′-dibenzo[a,d]cyclohepten]-3-yl)-1,2,5,6-tetrahydro-3-pyridinecarboxylate (isomer 4);

Ethyl 1-(5′,11′-dihydrospiro[cyclopentane-1,10′-dibenzo[a,d]cyclohepten]-3-yl)-4-piperidinecarboxylate (diastereomeric mixture 2);

Ethyl 1-(5′,11′-dihydrospiro[cyclopentane-1,10′-dibenzo[a,d]cyclohepten]-3-yl)-4-piperidinecarboxylate (isomer 2);

Ethyl 1-(5′,11′-dihydrospiro[cyclopentane-1,10′-dibenzo[a,d]cyclohepten]-3-yl)-4-piperidinecarboxylate (isomer 4);

Ethyl 1-(5′,11′-dihydrospiro[cyclopentane-1,10′-dibenzo[a,d]cyclohepten]-3-yl)-3-piperidinecarboxylate;

Ethyl 1-(5′,11′-dihydrospiro[cyclopentane-1,10′-dibenzo[a,d]cyclohepten]-3-yl)-3-piperidinecarboxylate (isomer 3);

Ethyl 1-(5′,11′-dihydrospiro[cyclopentane-1,10′-dibenzo[a,d]cyclohepten]-3-yl)-3-piperidinecarboxylate (isomer 4);

Ethyl 1-(5′,11′-dihydrospiro[cyclopentane-1,10′-dibenzo[a,d]cyclohepten]-3-yl)-3-piperidinecarboxylate (isomer 1);

Ethyl 1-(5′,11′-dihydrospiro[cyclopentane-1,10′-dibenzo[a,d]cyclohepten]-3-yl)-3-piperidinecarboxylate (isomer 2);

Ethyl 1-(2′-fluoro-11′-oxo-11′H-spiro[cyclopentane-1,10′-dibenzo[b,f]thiepin]-3-yl)-4-piperidinecarboxylate;

Ethyl 1-(5′,11′-dihydrospiro[cyclopentane-1,10′-dibenzo[a,d]cyclohepten]-3-yl)-4-fluoro-4-piperidinecarboxylate (diastereomeric mixture 2);

Ethyl 1-(5′,11′-dihydrospiro[cyclopentane-1,10′-dibenzo[a,d]cyclohepten]-3-yl)-4-fluoro-4-piperidinecarboxylate (isomer 4);

Ethyl 1-(5′,11′-dihydrospiro[cyclopentane-1,10′-dibenzo[a,d]cyclohepten]-3-yl)-4-fluoro-4-piperidinecarboxylate (isomer 2);

Methyl 1-(5′,11′-dihydrospiro[cyclopentane-1,10′-dibenzo[a,d]cyclohepten]-3-yl)-4-fluoro-4-piperidinecarboxylate (diastereomeric mixture 2);

Methyl 1-(5′,11′-dihydrospiro[cyclopentane-1,10′-dibenzo[a,d]cyclohepten]-3-yl)-4-fluoro-4-piperidinecarboxylate (isomer 2);

Methyl 1-(5′,11′-dihydrospiro[cyclopentane-1,10′-dibenzo[a,d]cyclohepten]-3-yl)-4-fluoro-4-piperidinecarboxylate (isomer 4);

Ethyl 4-(5′,11′-dihydrospiro[cyclopentane-1,10′-dibenzo[a,d]cyclohepten]-3-yl)-2-morpholinecarboxylate;

Ethyl 4-(5′,11′-dihydrospiro[cyclopentane-1,10′-dibenzo[a,d]cyclohepten]-3-yl)-2-morpholinecarboxylate (isomer 1);

Ethyl 4-(5′,11′-dihydrospiro[cyclopentane-1,10′-dibenzo[a,d]cyclohepten]-3-yl)-2-morpholinecarboxylate (isomer 4);

Ethyl 4-(5′,11′-dihydrospiro[cyclopentane-1,10′-dibenzo[a,d]cyclohepten]-3-yl)-2-morpholinecarboxylate (isomer 3);

Ethyl 4-(5′,11′-dihydrospiro[cyclopentane-1,10′-dibenzo[a,d]cyclohepten]-3-yl)-2-morpholinecarboxylate (isomer 2);

Ethyl 3-(5′,11′-dihydrospiro[cyclopentane-1,10′-dibenzo[a,d]cyclohepten]-3-yl)-3-azabicyclo[3.1.0]hexane-1-carboxylate;

Ethyl 3-(5′,11′-dihydrospiro[cyclopentane-1,10′-dibenzo[a,d]cyclohepten]-3-yl)-3-azabicyclo[3.1.0]hexane-1-carboxylate (isomer 3);

Ethyl 3-(5′,11′-dihydrospiro[cyclopentane-1,10′-dibenzo[a,d]cyclohepten]-3-yl)-3-azabicyclo[3.1.0]hexane-1-carboxylate (isomer 4);

Ethyl 3-(5′,11′-dihydrospiro[cyclopentane-1,10′-dibenzo[a,d]cyclohepten]-3-yl)-3-azabicyclo[3.1.0]hexane-1-carboxylate (isomer 1);

Ethyl 3-(5′,11′-dihydrospiro[cyclopentane-1,10′-dibenzo[a,d]cyclohepten]-3-yl)-3-azabicyclo[3.1.0]hexane-1-carboxylate (isomer 2);

Methyl 1-(11′H-spiro[cyclopentane-1,10′-dibenzo[b,f]oxepin]-3-yl)-1,2,5,6-tetrahydro-3-pyridinecarboxylate;

Methyl 1-(11′H-spiro[cyclopentane-1,10′-dibenzo[b,f]oxepin]-3-yl)-1,2,5,6-tetrahydro-3-pyridinecarboxylate (isomer 1);

Methyl 1-(11′H-spiro[cyclopentane-1,10′-dibenzo[b,f]oxepin]-3-yl)-1,2,5,6-tetrahydro-3-pyridinecarboxylate (isomer 2);

1-(11′H-spiro[cyclopentane-1,10′-dibenzo[b,t]oxepin]-3-yl)-1,2,5,6-tetrahydro-3-pyridinecarboxylic acid (isomer 1);

1-(11′H-spiro[cyclopentane-1,10′-dibenzo[b,t]oxepin]-3-yl)-1,2,5,6-tetrahydro-3-pyridinecarboxylic acid (isomer 2);

Methyl 1-(11′H-spiro[cyclopentane-1,10′-dibenzo[b,f]oxepin]-3-yl)-1,2,3,6-tetrahydro-4-pyridinecarboxylate;

Methyl 1-(11′H-spiro[cyclopentane-1,10′-dibenzo[b,f]oxepin]-3-yl)-1,2,3,6-tetrahydro-4-pyridinecarboxylate (isomer 1);

Methyl 1-(11′H-spiro[cyclopentane-1,10′-dibenzo[b,f]oxepin]-3-yl)-1,2,3,6-tetrahydro-4-pyridinecarboxylate (isomer 2);

1-(11′H-spiro[cyclopentane-1,10′-dibenzo[b,t]oxepin]-3-yl)-1,2,3,6-tetrahydro-4-pyridinecarboxylic acid (isomer 1);

1-(11′H-spiro[cyclopentane-1,10′-dibenzo[b,t]oxepin]-3-yl)-1,2,3,6-tetrahydro-4-pyridinecarboxylic acid (isomer 2);

Methyl 4-fluoro-1-(11′H-spiro[cyclopentane-1,10′-dibenzo[b,f]oxepin]-3-yl)-4-piperidinecarboxylate;

Methyl 4-fluoro-1-(11′H-spiro[cyclopentane-1,10′-dibenzo[b,f]oxepin]-3-yl)-4-piperidinecarboxylate (isomer 1);

Methyl 4-fluoro-1-(11′H-spiro[cyclopentane-1,10′-dibenzo[b,f]oxepin]-3-yl)-4-piperidinecarboxylate (isomer 2);

4-Fluoro-1-(11′H-spiro[cyclopentane-1,10′-dibenzo[b,f]oxepin]-3-yl)-4-piperidinecarboxylic acid (isomer 1);

4-Fluoro-1-(11′H-spiro[cyclopentane-1,10′-dibenzo[b,f]oxepin]-3-yl)-4-piperidinecarboxylic acid (isomer 2);

Methyl 1-(5′,11′-dihydrospiro[cyclopentane-1,10′-dibenzo[a,d]cyclohepten]-3-yl)-1,2,3,6-tetrahydro-4-pyridinecarboxylate;

Methyl 1-(5′,11′-dihydrospiro[cyclopentane-1,10′-dibenzo[a,d]cyclohepten]-3-yl)-1,2,3,6-tetrahydro-4-pyridinecarboxylate (isomer 1);

Methyl 1-(5′,11′-dihydrospiro[cyclopentane-1,10′-dibenzo[a,d]cyclohepten]-3-yl)-1,2,3,6-tetrahydro-4-pyridinecarboxylate (isomer 2);

1-(5′,11′-Dihydrospiro[cyclopentane-1,10′-dibenzo[a,d]cyclohepten]-3-yl)-1,2,3,6-tetrahydro-4-pyridinecarboxylic acid (isomer 2);

1-(5′,11′-Dihydrospiro[cyclopentane-1,10′-dibenzo[a,d]cyclohepten]-3-yl)-1,2,3,6-tetrahydro-4-pyridinecarboxylic acid (isomer 1);

Methyl 1-(5′,11′-dihydrospiro[cyclopentane-1,10′-dibenzo[a,d]cyclohepten]-3-yl)-3-azetidinecarboxylate;

Methyl 1-(5′,11′-dihydrospiro[cyclopentane-1,10′-dibenzo[a,d]cyclohepten]-3-yl)-3-azetidinecarboxylate (isomer 1);

Methyl 1-(5′,11′-dihydrospiro[cyclopentane-1,10′-dibenzo[a,d]cyclohepten]-3-yl)-3-azetidinecarboxylate (isomer 4);

Methyl 1-(5′,11′-dihydrospiro[cyclopentane-1,10′-dibenzo[a,d]cyclohepten]-3-yl)-3-azetidinecarboxylate (isomer 2);

Methyl 1-(5′,11′-dihydrospiro[cyclopentane-1,10′-dibenzo[a,d]cyclohepten]-3-yl)-3-azetidinecarboxylate (isomer 3);

1-(5′,11′-Dihydrospiro[cyclopentane-1,10′-dibenzo[a,d]cyclohepten]-3-yl)-3-azetidinecarboxylic acid formate salt (isomer 1);

1-(5′,11′-dihydrospiro[cyclopentane-1,10′-dibenzo[a,d]cyclohepten]-3-yl)-3-azetidinecarboxylic acid (isomer 1);

1-(5′,11′-Dihydrospiro[cyclopentane-1,10′-dibenzo[a,d]cyclohepten]-3-yl)-3-azetidinecarboxylic acid (isomer 2);

Methyl 1-(5′,11′-dihydrospiro[cyclopentane-1,10′-dibenzo[a,d]cyclohepten]-3-yl)-3-methyl-3-pyrrolidinecarboxylate;

Methyl 1-(5′,11′-dihydrospiro[cyclopentane-1,10′-dibenzo[a,d]cyclohepten]-3-yl)-3-methyl-3-pyrrolidinecarboxylate (isomer 1);

Methyl 1-(5′,11′-dihydrospiro[cyclopentane-1,10′-dibenzo[a,d]cyclohepten]-3-yl)-3-methyl-3-pyrrolidinecarboxylate (diastereomeric mixture 4);

1-(5′,11′-Dihydrospiro[cyclopentane-1,10′-dibenzo[a,d]cyclohepten]-3-yl)-3-methyl-3-pyrrolidinecarboxylic acid (isomer 1);

1-(5′,11′-Dihydrospiro[cyclopentane-1,10′-dibenzo[a,d]cyclohepten]-3-yl)-3-methyl-3-pyrrolidinecarboxylic acid (diastereomeric mixture 4);

Methyl 1-(5′,11′-dihydrospiro[cyclopentane-1,10′-dibenzo[a,d]cyclohepten]-3-yl)-3-pyrrolidinecarboxylate;

Methyl 1-(5′,11′-dihydrospiro[cyclopentane-1,10′-dibenzo[a,d]cyclohepten]-3-yl)-3-pyrrolidinecarboxylate (isomer 3);

Methyl 1-(5′,11′-dihydrospiro[cyclopentane-1,10′-dibenzo[a,d]cyclohepten]-3-yl)-3-pyrrolidinecarboxylate (isomer 4);

Methyl 1-(5′,11′-dihydrospiro[cyclopentane-1,10′-dibenzo[a,d]cyclohepten]-3-yl)-3-pyrrolidinecarboxylate (isomer 1);

Methyl 1-(5′,11′-dihydrospiro[cyclopentane-1,10′-dibenzo[a,d]cyclohepten]-3-yl)-3-pyrrolidinecarboxylate (isomer 2);

1-(5′,11′-Dihydrospiro[cyclopentane-1,10′-dibenzo[a,d]cyclohepten]-3-yl)-3-pyrrolidinecarboxylic acid (isomer 1);

1-(5′,11′-Dihydrospiro[cyclopentane-1,10′-dibenzo[a,d]cyclohepten]-3-yl)-3-pyrrolidinecarboxylic acid (isomer 2);

4-[5′,11′-Dihydrospiro[cyclopentane-1,10′-dibenzo[a,d]cyclohepten]-3-yl(methyl)amino]-2,2-dimethylbutanoic acid (diastereoisomer 1);

Methyl 4-(5′,11′-dihydrospiro[cyclopentane-1,10′-dibenzo[a,d]cyclohepten]-3-yl)-2-piperazinecarboxylate;

Methyl 4-(5′,11′-dihydrospiro[cyclopentane-1,10′-dibenzo[a,d]cyclohepten]-3-yl)-2-piperazinecarboxylate (isomer 1);

Methyl 4-(5′,11′-dihydrospiro[cyclopentane-1,10′-dibenzo[a,d]cyclohepten]-3-yl)-2-piperazinecarboxylate (isomer 2);

4-(5′,11′-Dihydrospiro[cyclopentane-1,10′-dibenzo[a,d]cyclohepten]-3-yl)-2-piperazinecarboxylic acid (isomer 1);

4-(5′,11′-Dihydrospiro[cyclopentane-1,10′-dibenzo[a,d]cyclohepten]-3-yl)-2-piperazinecarboxylic acid (isomer 2);

Methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (diastereomeric mixture 1);

Methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (diastereomeric mixture 2);

Methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (isomer 2);

Methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (isomer 4);

1-(2′-Fluoro-5′,11′-dihydrospiro[cyclopentane-1,10′-dibenzo[a,d]cyclohepten]-3-yl)-1,2,5,6-tetrahydro-3-pyridinecarboxylic acid (diastereomeric mixture 1);

1-(2′-Fluoro-5′,11-dihydrospiro[cyclopentane-1,10′-dibenzo[a,d]cyclohepten]-3-yl)-1,2,5,6-tetrahydro-3-pyridinecarboxylic acid (isomer 2);

Ethyl 1-(2′-chloro-11′-oxo-11′H-spiro[cyclopentane-1,10′-dibenzo[b,f]oxepin]-3-yl)-3-piperidinecarboxylate;

1-(2′-Chloro-11′-oxo-11′H-spiro[cyclopentane-1,10′-dibenzo[b,f]oxepin]-3-yl)-4-piperidinecarboxylic acid;

Ethyl 1-(2′-chloro-11′-oxo-11′H-spiro[cyclopentane-1,10′-dibenzo[b,f]oxepin]-3-yl)-3-piperidinecarboxylate;

1-(2′-Chloro-11′-oxo-11H-spiro[cyclopentane-1,10′-dibenzo[b,t]oxepin]-3-yl)-3-piperidinecarboxylic acid-hydrochloride;

Ethyl 1-(11′H-spiro[cyclopentane-1,10′-dibenzo[b,f]oxepin]-3-yl)-4-piperidinecarboxylate;

1-(11′H-Spiro[cyclopentane-1,10′-dibenzo[b,f]oxepin]-3-yl)-4-piperidinecarboxylic acid;

Ethyl (3R)-1-(11′H-spiro[cyclopentane-1,10′-dibenzo[b,f]oxepin]-3-yl)-3-piperidinecarboxylate;

(3R)-1-(11′H-spiro[cyclopentane-1,10′-dibenzo[b,f]oxepin]-3-yl)-3-piperidinecarboxylic acid;

Methyl 1-(5′,11′-dihydrospiro[cyclopentane-1,10′-dibenzo[a,d]cyclohepten]-3-yl)-4-hydroxy-4-piperidinecarboxylate;

Methyl 1-(5′,11′-dihydrospiro[cyclopentane-1,10′-dibenzo[a,d]cyclohepten]-3-yl)-4-hydroxy-4-piperidinecarboxylate (isomer 1);

Methyl 1-(5′,11′-dihydrospiro[cyclopentane-1,10′-dibenzo[a,d]cyclohepten]-3-yl)-4-hydroxy-4-piperidinecarboxylate (isomer 2);

Methyl 1-(5′,11′-dihydrospiro[cyclopentane-1,10′-dibenzo[a,d]cyclohepten]-3-yl)-4-hydroxy-4-piperidinecarboxylate (isomer 3);

Methyl 1-(5′,11′-dihydrospiro[cyclopentane-1,10′-dibenzo[a,d]cyclohepten]-3-yl)-4-hydroxy-4-piperidinecarboxylate (isomer 4);

1-(5′,11′-dihydrospiro[cyclopentane-1,10′-dibenzo[a,d]cyclohepten]-3-yl)-4-hydroxy-4-piperidinecarboxylic acid (isomer 1);

1-(5′,11′-Dihydrospiro[cyclopentane-1,10′-dibenzo[a,d]cyclohepten]-3-yl)-4-hydroxy-4-piperidinecarboxylic acid (isomer 2);

1-(5′,11′-Dihydrospiro[cyclopentane-1,10′-dibenzo[a,d]cyclohepten]-3-yl)-3-piperidinecarboxamide (isomer 2);

Ethyl-3-(5′,11′-dihydrospiro[cyclopentane-1,10′-dibenzo[a,d]cyclohepten]-3-yl)-3-azabicyclo[3.1.0]hexane-6-carboxylate (exo);

Ethyl-3-(5′,11′-dihydrospiro[cyclopentane-1,10′-dibenzo[a,d]cyclohepten]-3-yl)-3-azabicyclo[3.1.0]hexane-6-carboxylate (exo) (isomer 1);

Ethyl-3-(5′,11′-dihydrospiro[cyclopentane-1,10′-dibenzo[a,d]cyclohepten]-3-yl)-3-azabicyclo[3.1.0]hexane-6-carboxylate (exo) (isomer 2);

3-(5′,11′-Dihydrospiro[cyclopentane-1,10′-dibenzo[a,d]cyclohepten]-3-yl)-3-azabicyclo[3.1.0]hexane-6-carboxylic acid (exo) (isomer 1);

3-(5′,11′-Dihydrospiro[cyclopentane-1,10′-dibenzo[a,d]cyclohepten]-3-yl)-3-azabicyclo[3.1.0]hexane-6-carboxylic acid hydrochloride (exo) (isomer 2);

[1-(5′,11′-Dihydrospiro[cyclopentane-1,10′-dibenzo[a,d]cyclohepten]-3-yl)-3-pyrrolidinyl]acetic acid (diastereomeric mixture 3);

[1-(5′,11′-Dihydrospiro[cyclopentane-1,10′-dibenzo[a,d]cyclohepten]-3-yl)-3-pyrrolidinyl]acetic acid (isomer 2);

Ethyl 1-(5′,11′-dihydrospiro[cyclopentane-1,10′-dibenzo[a,d]cyclohepten]-3-yl)-4-methyl-4-piperidinecarboxylate;

Ethyl 1-(5′,11′-dihydrospiro[cyclopentane-1,10′-dibenzo[a,d]cyclohepten]-3-yl)-4-methyl-4-piperidinecarboxylate (isomer 1);

Ethyl 1-(5′,11′-dihydrospiro[cyclopentane-1,10′-dibenzo[a,d]cyclohepten]-3-yl)-4-methyl-4-piperidinecarboxylate (isomer 2);

1-(5′,11′-Dihydrospiro[cyclopentane-1,10′-dibenzo[a,d]cyclohepten]-3-yl)-4-methyl-4-piperidinecarboxylic acid (isomer 1);

1-(5′,11′-Dihydrospiro[cyclopentane-1,10′-dibenzo[a,d]cyclohepten]-3-yl)-4-methyl-4-piperidinecarboxylic acid (isomer 2);

Methyl 1-(11′H-spiro[cyclopentane-1,10′-dibenzo[b,f]oxepin]-3-yl)-3-azetidinecarboxylate;

Methyl 1-(11′H-spiro[cyclopentane-1,10′-dibenzo[b,f]oxepin]-3-yl)-3-azetidinecarboxylate (isomer 1);

Methyl 1-(11′H-spiro[cyclopentane-1,10′-dibenzo[b,f]oxepin]-3-yl)-3-azetidinecarboxylate (isomer 3);

Methyl 1-(11′H-spiro[cyclopentane-1,10′-dibenzo[b,f]oxepin]-3-yl)-3-azetidinecarboxylate (isomer 2);

1-(11′H-spiro[cyclopentane-1,10′-dibenzo[b,t]oxepin]-3-yl)-3-azetidinecarboxylic acid formic acid salt (isomer 1);

1-(11′H-Spiro[cyclopentane-1,10′-dibenzo[b,t]oxepin]-3-yl)-3-azetidinecarboxylic acid formic acid salt (isomer 2);

Methyl [1-(5′,11′-dihydrospiro[cyclopentane-1,10′-dibenzo[a,d]cyclohepten]-3-yl)-3-azetidinyl]acetate;

Methyl [1-(5′,11′-dihydrospiro[cyclopentane-1,10′-dibenzo[a,d]cyclohepten]-3-yl)-3-azetidinyl]acetate (isomer 1);

Methyl [1-(5′,11′-dihydrospiro[cyclopentane-1,10′-dibenzo[a,d]cyclohepten]-3-yl)-3-azetidinyl]acetate (isomer 2);

[1-(5′,11′-Dihydrospiro[cyclopentane-1,10′-dibenzo[a,d]cyclohepten]-3-yl)-3-azetidinyl]acetic acid formic acid salt (isomer 1);

[1-(5′,11′-Dihydrospiro[cyclopentane-1,10′-dibenzo[a,d]cyclohepten]-3-yl)-3-azetidinyl]acetic acid formic acid salt (isomer 2);

Methyl [1-(5′,11′-dihydrospiro[cyclopentane-1,10′-dibenzo[a,d]cyclohepten]-3-yl)-4-piperidinyl]acetate;

Methyl [1-(5′,11′-dihydrospiro[cyclopentane-1,10′-dibenzo[a,d]cyclohepten]-3-yl)-4-piperidinyl]acetate (isomer 1);

Methyl [1-(5′,11′-dihydrospiro[cyclopentane-1,10′-dibenzo[a,d]cyclohepten]-3-yl)-4-piperidinyl]acetate (isomer 3);

Methyl [1-(5′,11′-dihydrospiro[cyclopentane-1,10′-dibenzo[a,d]cyclohepten]-3-yl)-4-piperidinyl]acetate (isomer 2);

Methyl [1-(5′,11′-dihydrospiro[cyclopentane-1,10′-dibenzo[a,d]cyclohepten]-3-yl)-4-piperidinyl]acetate (isomer 4);

[1-(5′,11′-dihydrospiro[cyclopentane-1,10′-dibenzo[a,d]cyclohepten]-3-yl)-4-piperidinyl]acetic acid (isomer 1);

[1-(5′,11′-dihydrospiro[cyclopentane-1,10′-dibenzo[a,d]cyclohepten]-3-yl)-4-piperidinyl]acetic acid (isomer 2);

Methyl 1-(5′,11′-dihydrospiro[cyclopentane-1,10′-dibenzo[a,d]cyclohepten]-3-yl)-3-fluoro-3-piperidinecarboxylate (diastereomeric mixture 2);

Methyl 1-(5′,11′-dihydrospiro[cyclopentane-1,10′-dibenzo[a,d]cyclohepten]-3-yl)-3-fluoro-3-piperidinecarboxylate (isomer 2);

Methyl 1-(5′,11′-dihydrospiro[cyclopentane-1,10′-dibenzo[a,d]cyclohepten]-3-yl)-3-fluoro-3-piperidinecarboxylate (isomer 4);

(−) 1-(5′,11-Dihydrospiro[cyclopentane-1,10′-dibenzo[a,d]cyclohepten]-3-yl)-3-azetidinecarboxylic acid hydrochloride salt;

(−) 1-(5′,11-Dihydrospiro[cyclopentane-1,10′-dibenzo[a,d]cyclohepten]-3-yl)-3-azetidinecarboxylic acid; and

1-(5′,11′-dihydrospiro[cyclopentane-1,10′-dibenzo[a,d]cyclohepten]-3-yl)-3-fluoro-3- piperidinecarboxylic acid (isomer 2);

or a pharmaceutically acceptable salt thereof.

In a further embodiment, the compound of formula (I) is selected from the list consisting of:

(−) 1-(5′,11-Dihydrospiro[cyclopentane-1,10′-dibenzo[a,d]cyclohepten]-3-yl)-4-piperidinecarboxylic acid;

3-(5′,11′-Dihydrospiro[cyclopentane-1,10′-dibenzo[a,d]cyclohepten]-3-yl)-3-azabicyclo[3.1.0]hexane-1-carboxylic acid (isomer 2);

1-(5′,11′-Dihydrospiro[cyclopentane-1,10′-dibenzo[a, d]cyclohepten]-3-yl)-3-azetidinecarboxylic acid formate salt (isomer 1);

(−) 1-(5′,11-Dihydrospiro[cyclopentane-1,10′-dibenzo[a,d]cyclohepten]-3-yl)-3-azetidinecarboxylic acid hydrochloride salt;

(−) 1-(5′,11-Dihydrospiro[cyclopentane-1,10′-dibenzo[a,d]cyclohepten]-3-yl)-3-azetidinecarboxylic acid; and

1-(11′H-Spiro[cyclopentane-1,10′-dibenzo[b,f]oxepin]-3-yl)-3-azetidinecarboxylic acid formic acid salt (isomer 2);

or a pharmaceutically acceptable salt thereof.

In a preferred embodiment, the compound is 1-(5′,11′-Dihydrospiro[cyclopentane-1,10′-dibenzo[a,d]cyclohepten]-3-yl)-3-azetidinecarboxylic acid or a pharmaceutically acceptable salt thereof.

In a further preferred embodiment, the compound is (−) 1-(5′,11-Dihydrospiro[cyclopentane-1,10′-dibenzo[a,d]cyclohepten]-3-yl)-3-azetidinecarboxylic acid or a pharmaceutically acceptable salt thereof.

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents may be the same or different.

The compounds of formula (I) may form pharmaceutically or veterinarily acceptable salts, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, Hl, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. In addition, pharmaceutically acceptable base addition salts can be formed with a suitable inorganic or organic base such as triethylamine, ethanolamine, triethanolamine, choline, arginine, lysine or histidine, optionally in a suitable solvent such as an organic solvent, to give the base addition salt which is usually isolated for example by crystallisation and filtration. Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of one or more carboxylic acid moieties that may be present in the compound of formula (I). For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

Hereinafter, the compounds of formula (I) and their pharmaceutically acceptable salts, are referred to as “ the compounds of the invention”.

It will be appreciated by those skilled in the art that certain protected derivatives of the compounds of the invention, which may be made prior to a final deprotection stage, may not possess pharmacological activity as such, but may, in certain instances, be administered orally or parenterally and thereafter metabolised in the body to form compounds defined in the first aspect which are pharmacologically active. Such derivatives may therefore be described as “prodrugs”. All protected derivatives and prodrugs of compounds defined in the first aspect are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compound defined in the first aspect.

The compounds of the invention may exist in solvated or hydrated form.

The compounds of the invention or solvates/hydrates of the compounds or salts, may exist in one or more polymorphic forms.

Therefore, according to a further aspect, the invention includes a solvate, hydrate or prodrug of the compounds of the invention.

The compounds of the invention may exist in zwitterionic form.

Certain compounds of the invention may exist in one or more tautomeric forms. All tautomers and mixtures thereof are included in the scope of the present invention.

The compounds of the invention possess one or more chiral centres and so exist in a number of stereoisomeric forms. Compounds having one chiral centre may exist as enantiomers or a racemic mixture containing enantiomers. Compounds having two or more chiral centres may exist as diastereoismomers or enantiomers. All stereoisomers (for example enantiomers and diastereoisomers) and mixtures thereof are included in the scope of the present invention. Racemic mixtures may be separated to give their individual enantiomer using preparative HPLC using a column with a chiral stationary phase or resolved to yield individual enantiomers utilising methods known to those skilled in the art. In addition, chiral intermediate compounds may be resolved and used to prepare individual enantiomers.

The invention also includes all suitable isotopic variations of the compounds of the invention. An isotopic variation of the compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, sulphur, fluorine and chlorine such as ²H, ³H, ¹³C, ¹⁴C, ¹⁵N, ¹⁷O, ¹⁸O, ³⁵S, ¹⁸F and ³⁶Cl respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

Compounds of the invention may be prepared in a variety of ways. In the following reaction schemes and hereinafter, unless otherwise stated R¹ to R⁵, X, n and m are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic), etc . . . (IVa), (IVb), (IVc) etc.

Compounds of formula (Ia), i.e. compounds of general formula (I) where R⁵ is H and compounds of formula (Ib) where R⁵ is oxo, may be prepared according to reaction scheme 1 by reacting compounds of formula (II) or (IV) with compounds of formula NHR³R⁴ (III) in the presence of a suitable reducing agent (eg NaBH(OAc)₃) in an organic solvent (eg DCE) at room temperature for approximately 12 hours.

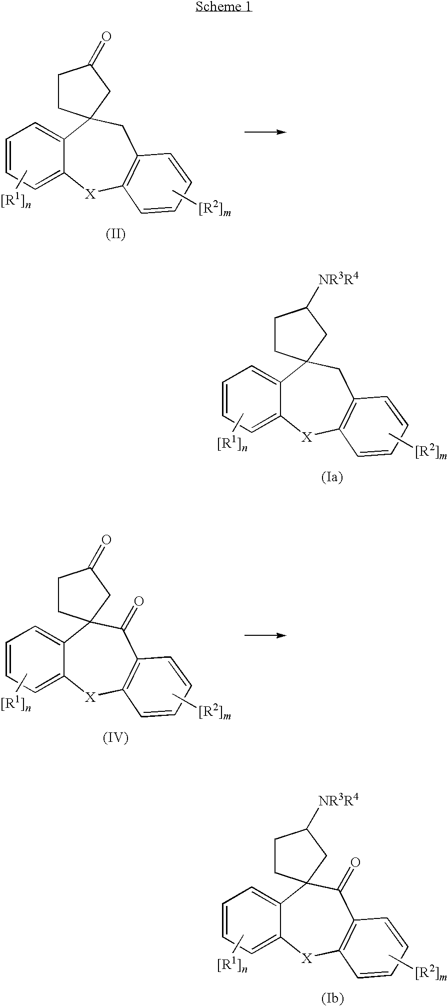

Compounds of formula (III) are either commercially available or may be prepared by procedures known to the skilled person.

Compounds of formula (II) may be prepared from compounds of formula (IV) according to reaction scheme 2 by reacting compounds of formula (IV) with gaseous hydrogen over a suitable catalyst (eg Pd/C) in a suitable organic solvent (eg THF/AcOH).

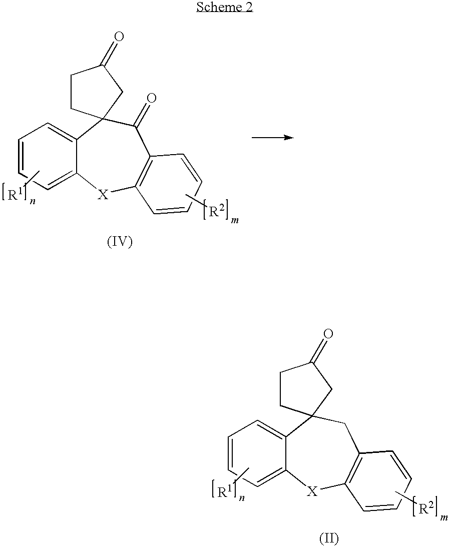

Compounds of formula (IV) may be prepared in two steps according to reaction scheme 3. Firstly compounds of formula (VI) are reacted with BH₃-THF followed by H₂O₂ oxidation in organic solvent (eg THF) at 0° C., to give compounds of formula (V). This mixture is then reacted with a suitable oxidizing agent (eg Dess Martin periodinane) at room temperature in DCM to give compounds of formula (IV).

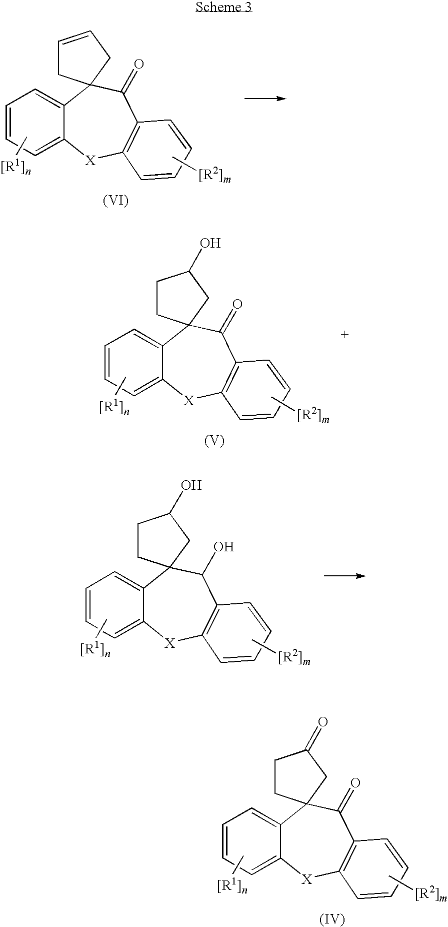

Compounds of formula (VI) may be prepared according to reaction scheme 4 by reacting compounds of formula (VII) with Grubb's 2^{nd} generation catalyst in organic solvent (eg DCM) at room temperature for approximately 6 hours.

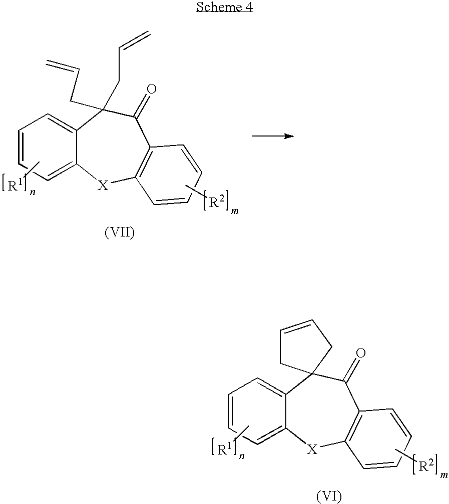

Compounds of formula (VII) may be prepared according to reaction scheme 5 by reacting compounds of formula (VIII) with a suitable base (eg potassium tert-butoxide) and an allyl halide in a suitable organic solvent (eg tBuOH) at 50° C. for approximately 6 hours.

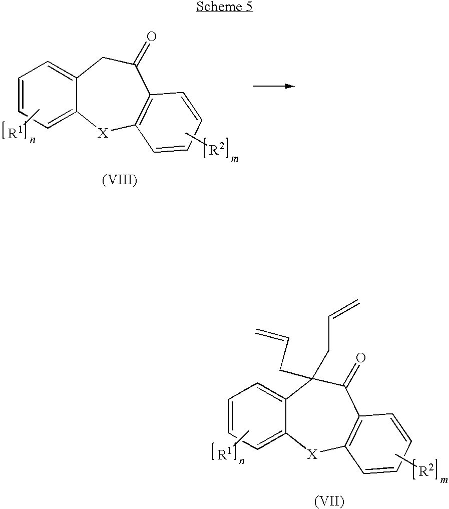

Compounds of formula (VIII) are either commercially available or may be prepared by procedures known to the skilled person (Lucini, V. et al., Journal of Medicinal Chemistry (2004), 47(17), 4202-4212; Trabanco, A. et Al., Chemical & Pharmaceutical Bulletin (2004), 52(2), 262-265).

The compounds of the invention are antagonists of the H₁ receptor . In addition, some of the compounds of the invention are antagonists of the 5HT_{2A} receptor.

The compounds of the invention are useful for the treatment of diseases and conditions mediated by antagonism of the H₁ receptor and optionally by antagonism of the 5HT_{2A} receptor.

Therefore, according to an embodiment, the invention provides the compounds of the invention for use as a medicament, preferably a human medicament.

The compounds of the invention may treat diseases or conditions selected from the list consisting of: [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Psychotic disorders for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9).

ii) Depression and mood disorders for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90).

iii) Anxiety disorders for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00).

iv) Substance-related disorders for example Substance Use Disorders (including Substance Dependence, Substance Craving and Substance Abuse); Substance-Induced Disorders (including Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders (including Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9)); Amphetamine (or Amphetamine-Like)-Related Disorders (for example Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9)); Caffeine Related Disorders (including Caffeine Intoxication (305.90), Caffeine-Induced Anxiety Disorder, Caffeine-Induced Sleep Disorder and Caffeine-Related Disorder Not Otherwise Specified (292.9)); Cannabis-Related Disorders (including Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9)); Cocaine-Related Disorders (including Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9)); Hallucinogen-Related Disorders (including Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9)); Inhalant-Related Disorders (including Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9)); Nicotine-Related Disorders (including Nicotine Dependence (305.1), Nicotine Withdrawal (292.0) and Nicotine-Related Disorder Not Otherwise Specified (292.9)); Opioid-Related Disorders (including Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9)); Phencyclidine (or Phencyclidine-Like)-Related Disorders (including Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9)); Sedative-, Hypnotic-, or Anxiolytic-Related Disorders (including Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic- Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9)); Polysubstance-Related Disorder (including Polysubstance Dependence (304.80)); and Other (or Unknown) Substance-Related Disorders (including Anabolic Steroids, Nitrate Inhalants and Nitrous Oxide).

v) Sexual dysfunction for example Sexual Desire Disorders (including Hypoactive Sexual Desire Disorder (302.71) and Sexual Aversion Disorder (302.79)); sexual arousal disorders (including Female Sexual Arousal Disorder (302.72) and Male Erectile Disorder (302.72)); orgasmic disorders (including Female Orgasmic Disorder (302.73), Male Orgasmic Disorder (302.74) and Premature Ejaculation (302.75)); sexual pain disorder (including Dyspareunia (302.76) and Vaginismus (306.51)); Sexual Dysfunction Not Otherwise Specified (302.70); paraphilias (including Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9)); gender identity disorders (including Gender Identity Disorder in Children (302.6) and Gender Identity Disorder in Adolescents or Adults (302.85)); and Sexual Disorder Not Otherwise Specified (302.9).

vi) Sleep disorder for example primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type).

vii) Eating disorders such as Anorexia Nervosa (307.1) (including the subtypes Restricting Type and Binge-Eating/Purging Type); Bulimia Nervosa (307.51) (including the subtypes Purging Type and Nonpurging Type); Obesity; Compulsive Eating Disorder; Binge Eating Disorder; and Eating Disorder Not Otherwise Specified (307.50).

viii) Autism Spectrum Disorders including Autistic Disorder (299.00), Asperger's Disorder, Rett's Disorder, Childhood Disintegrative Disorder and Pervasive Developmental Disorder Not Otherwise Specified.

ix) Attention-Deficit /Hyperactivity Disorder (including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9)); Hyperkinetic Disorder; Disruptive Behaviour Disorders such as Conduct Disorder (including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

x) Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

xi) Enhancement of cognition including the treatment of cognition impairment in other diseases such as schizophrenia, bipolar disorder, depression, other psychiatric disorders and psychotic conditions associated with cognitive impairment, e.g. Alzheimer's disease.

In an embodiment, the invention provides the use of the compounds of the invention in the manufacture of a medicament for treating or preventing sleep disorders.

In an embodiment the sleep disorder is selected from the list consisting of: primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type).

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with antidepressants to treat or prevent depression and mood disorders.

The compounds of the invention may be used in combination with the following agents to treat or prevent bipolar disease: i) mood stabilisers; ii) antipsychotics; and iii) antidepressants.

The compounds of the invention may be used in combination with the following agents to treat or prevent anxiety disorders: i) anxiolytics; and ii) antidepressants.

The compounds of the invention may be used in combination with the following agents to treat or prevent male sexual dysfunction: i) phosphodiesterase V inhibitors, for example vardenafil and sildenafil; ii) dopamine agonists/dopamine antagonists/dopamine transport inhibitors for example apomorphine and buproprion; iii) alpha adrenoceptor antagonists for example phentolamine; iv) prostaglandin agonists for example alprostadil; v) androgen receptor modulators such as testosterone; vi) serotonin agonists/antagonists/modulators/serotonin transporter inhibitors for example serotonin reuptake inhibitors; vii) noradrenaline transport inhibitors for example reboxetine; viii) oxytocin receptor antagonists; (ix) sodium and calcium channel inhibitors/blockers; and (x) opioid receptor antagonists.

The compounds of the invention may be used in combination with the same agents specified for male sexual dysfunction to treat or prevent female sexual dysfunction, and in addition an estrogen agonist such as estradiol.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine, sertraline femoxetine, fluvoxamine, indalpine and zimeldine); dual serotonin/noradrenaline reuptake inhibitors (such as venlafaxine, duloxetine and milnacipran); Noradrenaline reuptake inhibitors (such as reboxetine and venlafaxine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

It will be appreciated that the compound of the combination or composition may be administered simultaneously (either in the same or different pharmaceutical formulations), separately or sequentially.

It will be appreciated that references herein to “treatment” extend to prophylaxis, prevention of recurrence and suppression or amelioration of symptoms (whether mild, moderate or severe) as well as the treatment of established conditions.

The compound of the invention may be administered as the raw chemical but the active ingredient is suitably presented as a pharmaceutical formulation.

The compounds of the invention will normally, but not necessarily, be formulated into pharmaceutical compositions prior to administration to a patient by an appropriate route. Accordingly, in another aspect, the invention provides pharmaceutical compositions comprising a compound of the invention and one or more pharmaceutically-acceptable excipients.

As used herein, “pharmaceutically-acceptable excipient” means any pharmaceutically acceptable material present in the pharmaceutical composition or dosage form other than the compound or compounds of the invention. Typically the material gives form, consistency and performance to the pharmaceutical composition.

The pharmaceutical compositions of the invention typically contain one compound of the invention. However, in certain embodiments, the pharmaceutical compositions of the invention contain more than one compound of the invention. In addition, the pharmaceutical compositions of the invention may comprise one or more additional pharmaceutically active compounds.

Such pharmaceutical compositions of the invention may be prepared and packaged in bulk form wherein a safe and therapeutically effective amount of a compound of the invention can be dispensed and then given to the patient such as with powders or syrups. Alternatively, the pharmaceutical compositions of the invention may be prepared and packaged as dosage forms wherein each physically discrete dosage form contains a safe and effective amount of a compound of the invention. Accordingly, in another aspect, the invention provides dosage forms comprising pharmaceutical compositions of the invention.

A therapeutically effective amount of a compound of the present invention will depend upon a number of factors including, for example, the age and weight of the animal, the precise condition requiring treatment and its severity, the nature of the composition, and the route of administration, and will ultimately be at the discretion of the attendant physician or veterinarian. However, an effective amount of a compound of formula (I) for the treatment of disorders or diseases associated with H₁ antagonist activity will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt thereof may be determined as a proportion of the effective amount of the compound of formula (I) per se. It is envisaged that similar dosages would be appropriate for treatment of the other conditions referred to above.

It will be recognised by one of skill in the art that the optimal quantity and spacing of individual dosages of compounds of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of compounds of the invention given per day for a defined number of days, can be ascertained by those skilled in the art using conventional course of treatment determination tests.

The compositions of the invention will typically be formulated into dosage forms which are adapted for administration to the patient by the desired route of administration. For example, dosage forms include those adapted for (1) oral administration such as tablets, capsules, caplets, pills, lozenges, powders, syrups, elixirs, suspensions, solutions, emulsions, sachets and cachets; (2) parenteral administration such as sterile solutions, suspensions, implants and powders for reconstitution; (3) transdermal administration such as transdermal patches; (4) rectal and vaginal administration such as suppositories, pessaries and foams; (5) inhalation and intranasal such as dry powders, aerosols, suspensions and solutions (sprays and drops); (6) topical administration such as creams, ointments, lotions, solutions, pastes, drops, sprays, foams and gels; (7) ocular administration such as drops, ointment, sprays, suspensions and inserts; (8) buccal and sublingual administration such as lozenges, patches, sprays, drops, chewing gums and tablets.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance. Certain pharmaceutically-acceptable excipients may be chosen for their ability to facilitate the release of the compound of the invention at the appropriate rate to treat the condition.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavouring agents, flavour masking agents, colouring agents, anticaking agents, humectants, chelating agents, plasticizers, viscosity increasing agents, rate modifying agents, antioxidants, preservatives, stabilizers, surfactants and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to determine suitable pharmaceutically-acceptable excipients in appropriate amounts for use with the compounds of the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press). The pharmaceutical compositions of the invention may be prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch and pre-gelatinized starch), gelatin, acacia, sodium alginate, alginic acid, tragacanth, guar gum, povidone, and cellulose and its derivatives (e.g. hydroxypropyl methyl cellulose). The oral solid dosage form may further comprise a disintegrant. Suitable disintegrants include starches, crospovidone, sodium starch glycolate, cros-carmellose, alginic acid, and sodium carboxymethyl cellulose. The oral solid dosage form may further comprise a lubricant. Suitable lubricants include stearic acid, magnesium stearate, calcium stearate, and sodium dodecyl sulphate. The oral solid dosage form may further comprise a glidant such as talc and colloidal silicon dioxide. The oral solid dosage form may further comprise an outer coating which may have cosmetic or functional properties.

It will be appreciated that the invention includes the following further aspects. The diseases and conditions described above extend, where appropriate, to these further aspects.
i) A compound of the invention for use in treating or preventing sleep disorders.
ii) A method of treatment or prevention of sleep disorders in a mammal comprising administering an effective amount of a compound of the invention.

### SUPPORTING COMPOUNDS AND INTERMEDIATES

The invention is illustrated by the Compounds described below.

In the procedures that follow, after each starting material, reference to an intermediate is typically provided. This is provided merely for assistance to the skilled chemist. The starting material may not necessarily have been prepared from the batch referred to.

Compounds were named using ACD/Name PRO 6.02 chemical naming software (Advanced Chemistry Development Inc., Toronto, Ontario, M5H2L3, Canada).

Reagents were obtained from commercial suppliers (for example Sigma-Aldrich and Lancaster) and used without further purification. Solvents were obtained in dry form or were dried according to standard procedures. For example, DCM and DCE were dried over calcium hydride; THF, toluene and diethyl ether were dried over Na/benzophenone; and EtOH was dried over Mg/I₂. Anhydrous reactions were run under a positive pressure of dry N₂ or argon.

Proton Nuclear Magnetic Resonance (¹H NMR) spectra were recorded either on Varian instruments at 300, 400, 500 or 600 MHz, or on Bruker instruments at 300, 400 or 500 MHz. Chemical shifts are reported in ppm (δ) using the residual solvent line as internal standard. Splitting patterns are designated as: s, singlet; d, doublet; t, triplet; q, quartet; m, multiplet; b, broad. The NMR spectra were recorded at a temperature ranging from 25 to 90° C.

Mass spectra (MS) were run on a 4 II triple quadrupole Mass Spectrometer on an Agilent MSD 1100 Mass Spectrometer, operating in ES(+) and ES(−) ionization mode. The usage of this methodology is indicated by “MS”.

Optical rotations were measured by using a Jasco DIP-360 digital polarimeter with a path length of 10 cm recorded at the sodium D line.

HPLC-Mass spectra (HPLC-MS) were run on an Agilent LC/MSD 1100 Mass Spectrometer, operating in ES(+) and ES(−) ionization mode coupled with HPLC instrument Agilent 1100 Series [LC/MS−ES (+): analysis performed on a Supelcosil ABZ+Plus (33×4.6 mm, 3 m) (mobile phase: 100% [water+0.1% formic acid] for 1 min, then from 100% [water+0.1% formic acid] to 5% [water+0.1% formic acid] and 95% [acetonitrile] in 5 min, finally under these conditions for 2 min; T=40° C.; flow=1 mL/min; LC/MS−ES (−): analysis performed on a Supelcosil ABZ+Plus (33×4.6 mm, 3 m) (mobile phase: 100% [water+0.05% ammonia] for 1 min, then from 100% [water+0.05% ammonia] to 5% [water+0.05% ammonia] and 95% [acetonitrile] in 5 min, finally under these conditions for 2 min; T=40° C.; flow=1 mL/min]. In the mass spectra only one peak in the molecular ion cluster is reported. The usage of this methodology is indicated by “HPLC-MS” in the analytical characterization of the described compounds.

Alternatively mass directed analytical HPLC (Agilent technology HP1100) was carried out using a 19 mm×100 mm or 30 mm×100 mm, 5 μm, reversed phase Waters Atlantis column as the stationary phase and a gradient from water+0.1% formic acid to acetonitrile+0.1% formic acid as the eluent. The HPLC system was monitored by DAD array detector and an Agilent 110MSD mass spectrometer. The LC elution method (using Zorbax Eclipse XDB, 4.6×150 mm, 5 μm C8 column) was the following: 15 min method at 25° C., mobile phase composed of different CH3CN/H2O—HCOOH 0.1% mixtures at a flow rate of 1 mL/min (all solvent were HPLC grade, Fluka).

Alternatively HPLC spectra were performed using a reversed-phase liquid chromatography (ProStar 210/215 PrepStar218) and UV-Vis Detector (ProStar 325). The LC elution method (using Varian Polaris 5 C-18, 150×4.6 mm) was the following: 15 min method at 25° C., mobile phase composed of different CH₃CN/H₂O-HCOOH 0.1% mixtures at a flow rate of 1 mL/min (all solvent were HPLC grade, Fluka).

Alternatively HPLC spectra were performed using a Waters 2690 apparatus at 25° C. using a 3 mm×100 mm, 3.5 μm, reversed phase X-Terra C-18 column as the stationary phase and a gradient from water+0.1% formic acid 5% to acetonitrile+0.1% formic acid 90% during 19.5 min or water+0.1% formic acid 20% to acetonitrile+0.1% formic 95% during 19 min as the eluent. Flow rate was 0.5 mL/min (all solvents were HPLC grade, Merck). The HPLC system was monitored by DAD array detector at 254 nm and a Micromass Quattromicro mass spectrometer.

Total ion current (TIC) and DAD UV chromatographic traces together with MS and UV spectra associated with the peaks were taken also on a UPLC/MS Acquity™ system equipped with 2996 PDA detector and coupled to a Waters Micromass ZQ™ mass spectrometer operating in positive or negative electrospray ionisation mode. [LC/MS−ES (+/−): analyses performed using an Acquity™ UPLC BEH C18 column (50×21 mm, 1.7 μm particle size), column temperature 40° C. (mobile phase: A-water+0.1% formic acid/B−acetonitrile+0.075% formic acid, Flow rate: 1.0 mL/min, Gradient: t=0 min 3% B, t=0.05 min 6% B, t=0.57 min 70% B, t=1.4 min 99% B, t=1.45 min 3% B)]. The usage this methodology is indicated by “UPLC-MS” in the analytic characterization of the described compounds.

GC-MS (Varian Saturn 2000) was carried out using a Varian Chrompack CP-Sil Low bleed\MS 30 m×0.25 mm, 0.5 μm column as the stationary phase and helium (2mL/min) as the carrier gas. Injector temperature was 270° C., column temperature was increased from 200° C. to 300° C. at a rate of 10° C/min and then held at 300° C. for 5 min. Mass detection was performed using chemical ionization (CH₃CN) in the range from 200m/z to 450 m/z.

For reactions involving microwave irradiation, a Personal Chemistry Emrys™ Optimizer was used.

Flash silica gel chromatography was carried out on silica gel 230-400 mesh (supplied by Merck AG Darmstadt, Germany) or over Varian Mega Be—Si pre-packed cartridges or over pre-packed Biotage or Isolute Flash™ silica cartridges. Alternatively chromatographic purifications were performed on columns packed with Merck 60 silica gel, 23-400 mesh, for flash technique. Thin-layer chromatography was carried out using Merck TLC plates Kieselgel 60E-254, visualised with UV light, 5% phosphomolybdic acid, aqueous potassium permanganate. SPE-SCX cartridges are ion exchange solid phase extraction columns by supplied by Varian. The eluent used with SPE-SCX cartridges is methanol followed by 2N ammonia solution in methanol. Oasis® HLB extraction cartridges are ion exchange solid phase extraction columns by supplied by Waters. The eluent used with HLB cartridges is water followed by methanol.

In a number of preparations, purification was performed using either Biotage manual flash chromatography (Flash+) or automatic flash chromatography (Horizon) systems. All these instruments work with standard Biotage Silica cartridges.

In a number of preparations, purification was performed on a Mass-Directed Autopurification (MDAP) system Fraction Lynx™ equipped with Waters 2996 PDA detector and coupled with a ZQ™ mass spectrometer (Waters) operating in positive and negative electrospray ionisation mode ES+, ES− (mass range 100-1000).

A set of acidic as well as basic semi-preparative gradients have been used:

METHOD A: Chromatographic Acidic conditions for up to 30 mq of crude:

Column: 100×21.2 mm Supelcosil™ ABZ+Plus (5 μm particle size)

Mobile phase: A[water+0.1% formic acid]/B[acetonitrile+0.1% formic acid]

Flow rate: 20 mL/min

Gradient: 5% B for 1 min, 95% B in 9 min, 100% B in 3.5 min

METHOD B: Chromatographic Acidic conditions for up to 100 mq of crude:

Column: 150×30 mm XTerra Prep MS C18 (10 μm particle size)

Mobile phase: A[water+0.1% formic acid]/B [acetonitrile+0.1% formic acid]

Flow rate: 40 mL/min

Gradient: 1% B to 100% B in 7 min lasting for 7.5 min.

METHOD C: Chromatographic Basic conditions for up to 100 mg of crude

Column: 150×30 mm XTerra Prep MS C18 (10 μm particle size)

Mobile phase: A-water+10 mM ammonium carbonate (adjusted to pH 10 with ammonia)/B−acetonitrile

Flow rate: 40 mL/min

Gradient: 10% B for 0.5 min, 95% B in 12.5 min

Abbreviations

The following lists the abbreviations used:
DCM Dichloromethane
DCE Dichloroethane
THF Tetrahydrofuran
DMF Dimethylformamide
PPA Polyphosphoric acid
DMSO-d₆ Dimethyl sulfoxide-d₆
cHex Cyclohexane
BOC₂O Si-tert-butyl dicarbonate
SCX Strong cation resin
TEA Triethyl amine
TFA Trifluoro acetic acid
AcOH Acetic acid
e.e. Enantiomeric excess
d.e. Diastereoisomeric excess

Nomenclature

From reductive amination reaction (Scheme 1 in the description) of racemic Intermediate 5, 20, 35, 15 or 19 with chiral or achiral amines (or derivatives), 4 products may be obtained:
2 diastereoisomers in a ratio usually comprised between 70/30 and 90/10 and the corresponding enantiomers (e.g. Compound 70).

The major diastereoisomer is named diastereoisomer 1, the minor diastereoisomer is named diastereoisomer 2.

In order to name the corresponding enantiomers of the 2 diastereisomers it has been decided to use the terms enantiomer 1 or enantiomer 2 depending on the retention time in the corresponding chiral HPLC separation (e.g. Compound 70). The term enantiomer 1 is used for the single stereoisomer with the minor retention time in the condition of the chiral separation. Conversely the term enantiomer 2 is used for the single stereoisomer with the major retention time in the condition of the chiral separation.

An exemplary scheme is provided starting from Intermediate 5:

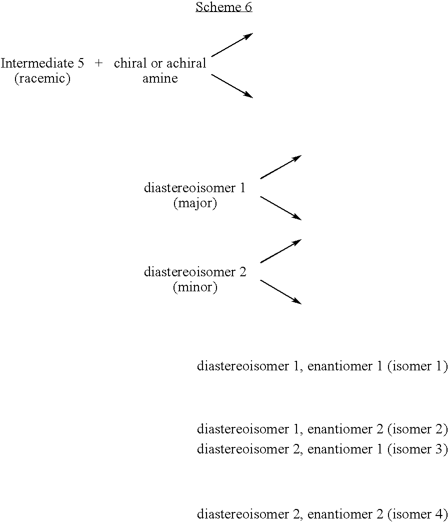

From reductive amination reaction (Scheme 1 in the description), of Intermediate 6, 7 or Intermediate 36, 37 in enantiomeric form with racemic amines (or derivatives), 4 products may be obtained:
2 diastereoisomers in a ratio usually comprised between 70/30 and 90/10 and the corresponding enantiomers (e.g. Compound 45).

The major diastereoisomer is named diastereoisomer 1, the minor diastereoisomer is named diastereoisomer 2.

The nomenclature adopted in this case is the same as discussed before: the term enantiomer 1 is used for the single stereoisomer with the minor retention time in the condition of the chiral separation. Conversely the term enantiomer 2 is used for the single stereoisomer with the major retention time in the condition of the chiral separation. An exemplary scheme is provided starting from Intermediate 6.

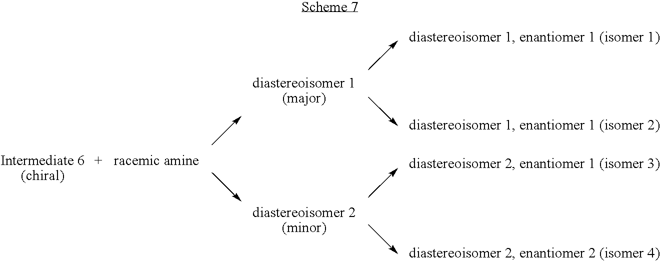

For both Scheme 6 and Scheme 7 it is assumed that:
in case (diasteroisomer 1, enantiomer 1) and (diastereisomer 2, enantiomer 1) are not present as single isomers, they will be named diastereoisomeric mixture 1;
in case (diasteroisomer 1, enantiomer 2) and (diastereisomer 2, enantiomer 2) are not present as single isomers, they will be named diastereoisomeric mixture 2;
in case (diasteroisomer 1, enantiomer 1), (diastereisomer 2, enantiomer 1) (diastereisomer 2, enantiomer 2) are not present as single isomers, they will be named diastereoisomeric mixture 3;
in case (diasteroisomer 1, enantiomer 2), (diastereisomer 2, enantiomer 1) (diastereisomer 2, enantiomer 2) are not present as single isomers, they will be named diastereoisomeric mixture 4.

For the reader's benefit the term:

(diastereoisomer 1, enantiomer 1) will be named from now on isomer 1,

(diastereoisomer 1, enantiomer 2) will be named from now on isomer 2;

(diastereoisomer 2, enantiomer 1) will be named from now on isomer 3;

(diastereoisomer 2, enantiomer 2) will be named from now on isomer 4.

From reductive amination reaction of enantiomeric Intermediate 6, 7 or Intermediate 36, 37 with chiral or achiral amines (or derivatives), 2 products may be obtained:
2 diastereoisomers (single or mixture) in a ratio usually comprised between 70/30 and 90/10 (e.g. Compound 21 and Compound 22).

An exemplary scheme is provided starting from Intermediate 6 and 7:

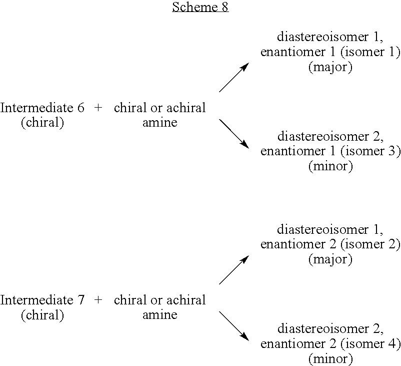

The nomenclature adopted for this case is according to the previous one applied for cases as in Scheme 6 and Scheme 7.

### Intermediate 1: 11,11-Di-2-propen-1-yl-5,11-dihydro-10H-dibenzo[a,d]cyclohepten-10-one

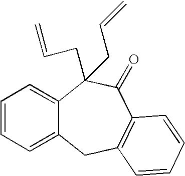

A solution of potassium tert-butoxide was prepared by dissolving potassium (0.094 g, 2.4 mmol) in a mixture of (12 mL) t-BuOH and dry toluene(3 mL). To this solution were added 5,11-dihydro-10H-dibenzo[a]cyclohepten-10-one (0.200 g, 0.96 mmol, whose preparation has been described in J. Med. Chem. 2004, 47, 4202-4212) and allyl bromide (0.23 mL, 2.7 mmol). The reaction was then heated to 55-60° C. for 1 hour. After cooling, saturated NaHCO₃ solution was added. The mixture was stirred for 15 minutes at room temperature and the aqueous phase was then extracted using diethyl ether. Major impurities were removed by column chromatography after which the compound was crystallized from methanol affording 196 mg of the title compound; MS (ESI) m/z: 311 [M+Na]⁺; ¹HNMR (CDCl₃): δ 2.75-2.96 (m, 4H), 3.94 (s, 2H), 4.92-5.04 (m, 4H), 5.42-5.55 (m, 2H), 7.10-7.35 (m, 8H) (7032-18-03).

### Intermediate 2: Spiro[cyclopent-3-ene-1,10′-dibenzo[a,d]cyclohepten]-11′(5′H)-one

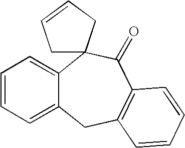

To a solution of 11,11-di-2-propen-1-yl-5,11-dihydro-10H-dibenzo[a]cyclohepten-10-one (Intermediate 1, 1.00 g, 3.47 mmol) in degassed DCM (1 L) was added 2^{nd} generation Grubbs catalyst (15mol %, 0.44 g) under Argon atmosphere at room temperature. The reaction mixture was stirred at room temperature overnight. The dark solution was then adsorbed on silica gel (10 eq wt relative to catalyst) and passed through a pad of silica gel (petroleum ether/diethyl ether 1/1). The filtered solution was stirred with activated charcoal (50 eq wt relative to product) for 12 h. After the carbon was filtered, the filtrate was concentrated in vacuo and purified by silica gel column chromatography (petroleum ether/diethyl ether=9/1) to provide 748 mg of the title compound as white solid; MS (ESI) m/z: 283 [M+Na]⁺; ¹HNMR (CDCl₃): δ 2.92-2.99 (m, 2H), 3.56-3.63 (m, 2H), 4.36 (m, 2H), 5.71-5.74 (m, 2H), 7.09-7.51 (m, 7H), 7.78-7.88 (m, 1H).

### Intermediate 3: Mixture of 3-hydroxyspiro[cyclopentane-1,10′-dibenzo[a,d]cyclohepten]-11′(5′H)-one and 5′,11-dihydrospiro[cyclopentane-1,10′-dibenzo[a,d]cycloheptene]-3,11′-diol

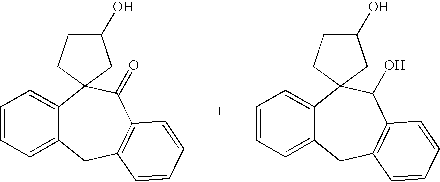

1M Borane solution in THF (0.77 mL, 0.77 mmol) was added dropwise to a stirred solution of spiro[cyclopent-3-ene-1,10′-dibenzo[a,d]cyclohepten]-11′(5′H)-one (Intermediate 2, 0.200 g, 0.77 mmol) in anhydrous THF (1.6 mL) at room temperature under an N₂ atmosphere, and the mixture was stirred at room temperature for 2.5 hours. Water (0.08 mL) was then added dropwise, followed by 3M sodium hydroxide (0.10 mL). Hydrogen peroxide (0.12 mL, 35%) was then added at a rate to maintain the temperature between 30 and 50° C., and the reaction mixture was stirred for 16 hours at room temperature. Diethyl ether (1.6 mL) was added to the reaction mixture and the organic phase was washed with brine and water. The organic solvent was evaporated to give a residue which, for analytical purpose, was purified by silica gel column chromatography (5/1 petroleum ether/diethyl ether) affording 3-hydroxyspiro[cyclopentane-1,10′-dibenzo[a,d]cyclohepten]-11′(5′H)-one (0.091 g) and 5′,11′-dihydrospiro[cyclopentane-1,10′-dibenzo[a,d]cycloheptene]-3,11′-diol (0.079 g).

3-hydroxyspiro[cyclopentane-1,10′-dibenzo[a,d]cyclohepten]-11′(5′H)-one:

MS (ESI) m/z: 279 [M+1]⁺, 301 [M+Na]⁺; 261 [M−H₂O]⁺; 579 [2M+NA]⁺; ¹HNMR (CDCl₃): δ 1.66-1.92 (m, 2H), 2.12-2.21 (m, 1H), 2.32-2.48 (m, 1H), 2.85-2.95 (m, 1H), 3.21-3.30 (m, 1H), 4.35-4.44 (m, 3H), 7.10-7.46 (m, 7H), 7.91-7.95 (m, 1H),

5′,11′-dihydrospiro[cyclopentane-1,10′-dibenzo[a,d]cycloheptene]-3,11′-diol:

MS (ESI) m/z: 303 [M+Na]⁺; ¹HNMR (CDCl₃): δ 1.83-2.43 (m, 5H), 2.61-2.71 (m, 1H), 3.81-3.89 (m, 1H), 4.47-4.61 (m, 2H), 5.03 (s, 1H), 7.03-7.61 (m, 8H).

### Intermediate 4: 3H-Spiro[cyclopentane-1,10′-dibenzo[a,d]cycloheptene]-3,11′(5′H)-dione

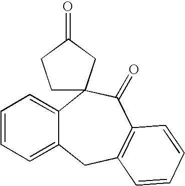

To a solution of Dess-Martin triacetoxyperiodinane (0.38 g, 0.9 mmol) a mixture of 3-hydroxyspiro[cyclopentane-1,10′-dibenzo[a,d]cyclohepten]-11′(5′H)-one and 5′,11′-dihydrospiro[cyclopentane-1,10′-dibenzo[a,d]cycloheptene]-3,11′-diol (Intermediate 3, 0.100 g, 0.36 mmol) was added in dry DCM (8 mL). The reaction mixture was left at 25° C. for 3.5 hours. The reaction was diluted with DCM (10 mL) and washed with NaOH (1N) and then brine. The organic layer was dried over Na₂SO₄ and after solvent evaporation gave 92 mg of the title compound; MS (ESI) m/z: 299 [M+Na]⁺; ¹HNMR (CDCl₃): δ 2.26-2.49 (m, 3H), 2.76-2.84 (m, 1H), 3.26-3.46 (m, 2H), 4.32-4.51 (m, 2H), 7.16-7.48 (m, 7H), 7.92-7.96 (m, 1H).

### Intermediate 5: 5′,11′-Dihydro-3H-spiro[cyclopentane-1,10′-dibenzo[a,d]cyclohepten]-3-one

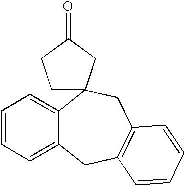

In a Parr apparatus, 3H-spiro[cyclopentane-1,10′-dibenzo[a,d]cycloheptene]-3,11′(5′H)-dione (Intermediate 4, 2.200 g, 7.96 mmol) was dissolved in THF (40 mL). AcOH (10.00 mL) and wet Pd/C (10% (w/w), 50% w/w of water content) (4.24 g, 3.98 mmol) were added and the mixture was hydrogenated (0.016 g, 7.96 mmol) under 5 atmospheres pressure for 5 days. During this time three portions of 2.5 g of Pd/C (10% (w/w), 50% w/w of water content) were added. The palladium was then filtered over celite and the solvent evaporated to afford 2.2 g of the title compound as a racemic mixture; MS (ESI) m/z: 285 [M+Na]⁺; ¹HNMR (CDCl₃): δ 2.24-2.32 (m, 2H), 2.51-2.62 (m, 4H), 3.09-3.16 (m, 2H), 4.11-4.21 (m, 2H), 7.04-7.35 (m, 8H).

The racemic mixture of 5′,11′-dihydro-3H-spiro[cyclopentane-1,10′-dibenzo[a]cyclohepten]-3-one (Intermediate 5) was submitted for preparative chiral HPLC (Column: Chiralpak IA (25×2.0 cm), 5 u; Mobile phase: n-Hexane/(Ethanol/Methanol 50/50) 96/4% v/v; Flow rate: 14 mL/min; UV: 225 nm; 19 mg/inj in CH₂Cl₂/Ethanol/Methanol/ Hexane) and give two enantiomers:

### Intermediate 6: (+) 5′,11′-Dihydro-3H-spiro[cyclopentane-1,10′-dibenzo[a,d]cyclohepten]-3-one

[α]_{D}²⁰=−83° (c=0.88, CHCl₃) (optical rotation was measured on a different batch);

retention time =12.5 min (683 mg)

### Intermediate 7: (+) 5′,11′-Dihydro-3H-spiro[cyclopentane-1,10′-dibenzo[a,d]cyclohepten]-3-one

[α]_{D}²⁰=+83° (c=0.93, CHCl₃) (optical rotation was measured on a different batch);

retention time =14.0 min (655 mg).

### Intermediate 8: Methyl N-(5′,11-dihydrospiro[cyclopentane-1,10′-dibenzo[a,d]cyclohepten]-3-yl)-β-alaninate (diastereomeric mixture 1)

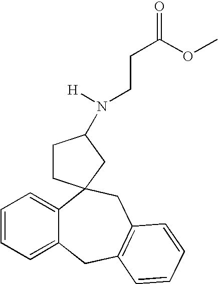

To a solution of (−) 5′,11′-dihydro-3H-spiro[cyclopentane-1,10′-dibenzo[a]cyclohepten]-3-one (Intermediate 6, 45 mg) in MeOH/DCM (1:1, 3 mL) was added methyl β-alaninate HCl salt (28.7 mg). DIPEA (36 μL, 0.21 mmol) was then added and the reaction mixture was left stirring for 15 mins until dissolution of the amino ester occurred. AcOH (315 mg) was added and the reaction mixture was left to stir for 2 h. Solid NaCNBH₃ (16 mg) was added portion-wise and the reaction left to stir overnight. Further NaCNBH₃ (11 mg, 0.171 mmol) was added and the mixture left stirring for another 24 h (48 h overall). The solvent was then evaporated, and the residue was dissolved in DCM, washed with saturated aqueous NaHCO₃ and dried over Na₂SO₄. The residue was dissolved in DCM, purified by SCX (eluting with a 1/1 mixture of 2.0M solution of NH₃ in MeOH/DCM) to afford 21 mg of the title product as mixture of diastereoisomers; UPLC/MS Rf=0.62; m/z (ES): 350.1 [M+H]⁺.

### Intermediate 9: Methyl N-(5′,11′-dihydrospiro[cyclopentane-1,10′-dibenzo[a,d]cyclohepten]-3-yl)-β-alaninate (diastereomeric mixture 2)

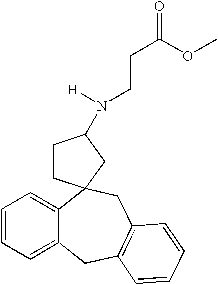

To a solution of (+) 5′,11′-dihydro-3H-spiro[cyclopentane-1,10′-dibenzo[a,d]cyclohepten]-3-one (Intermediate 7, 50 mg) in MeOH/DCM (1:1, 3 mL) was added methyl β-alaninate HCl salt (31.9 mg). DIPEA (40 μL, 0.23 mmol) was then added and the reaction was left stirring for 15 mins until complete dissolution of the amino ester occurred. AcOH (525 mg) was added and the reaction was left stirring for 2 h. Solid NaCNBH₃ (18 mg) was added portion-wise and the reaction left to stir overnight. Further NaCNBH₃ (11 mg, 0.171 mmol) was added and the reaction left to stir for another 24 h (48 h overall). The solvent was then evaporated, the residue was dissolved in DCM, washed with saturated aqueous NaHCO₃ solution and dried over Na₂SO₄. The residue was dissolved in DCM, purified by SCX (eluting with MeOH and with a 1/1 mixture 2.0M solution of NH₃ in MeOH/DCM) to afford 30 mg of the desired product as mixture of diastereoisomers; UPLC/MS Rf=0.64; m/z (ES): 350.1 [M+H]+.

### Intermediate 10: Methyl 4-[5′,11′-dihydrospiro[cyclopentane-1,10′-dibenzo[a,d]cyclohepten]-3-yl(methyl)amino]butanoate (diastereomeric mixture 1)

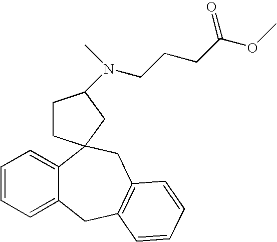

To a solution of N-methyl-5′,11-dihydrospiro[cyclopentane-1,10′-dibenzo[a,d]cyclohepten]-3-amine (diastereomeric mixture 1, Compound 16, 45 mg) in DCM/MeOH (5 mL, 1/1 ratio) was added methyl 4-oxobutanoate (0.024 mL, 0.227 mmol). AcOH (0.279 mL, 4.87 mmol) and NaCNBH₃ (15.29 mg, 0.243 mmol) were added and the reaction was left overnight. Solvents were removed under reduced pressure and the residue was dissolved in DCM, and washed with saturated aqueous solution of NaHCO₃. The organic phase was filtered and the solvent evaporated. The crude mixture was purified using a SCX cartridge (eluting with a 2M NH₃ solution in MeOH). The MeOH was removed, the residue was dissolved in DCM, and isocyanate resin (1.67 mmol/g) (100 mg) was added to remove excess secondary amine. This mixture was left under stirring overnight. After filtration and solvent evaporation 40 mg of the title product was obtained; UPLC/MS Rf=0.67; m/z (ES): 378.2 [M+H]⁺; ¹H NMR (400 MHz, CHLOROFORM-d) d ppm 6.98-7.54 (m, 8H) 3.98-4.27 (m, 2H) 3.65-3.76 (m, 3H) 2.98-3.37 (m, 3H) 2.50-2.73 (m, 2H) 2.32-2.50 (m, 5H) 1.83-2.31 (m, 8H).

### Intermediate 11: Mixture of 2-fluoro-10-(2-propen-1-yl)-11-(2-propen-1-yloxy)dibenzo[b,f]thiepin and 8-fluoro-11,11-di-2-propen-1-yldibenzo[b,f]thiepin-10(11H)-one

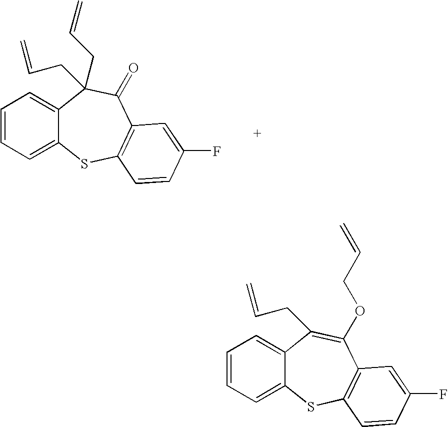

To a solution of potassium tert-butoxide (12.37 g, 110 mmol) in tBuOH (500 mL) were added 8-fluoro-11,11-di-2-propen-1-yldibenzo[b,f]thiepin-10(11H)-one (5 g, 15.4 mmol, see Collect. Czech. Chem. Commun. 1968, 33, 1831-1845) and allyl bromide (9.31 mL, 109.2 mmol). The reaction mixture was heated at 60° C. for 4 hours. After cooling, saturated aqueous NaHCO₃ solution (750 mL) was added in one portion. The mixture was left to stir for 15 minutes at room temperature. The precipitate was filtered off and the aqueous phase was extracted using ethyl-acetate. Collected organic layers were washed with saturated NaHCO₃ solution, dried with anhydrous Na₂SO₄ and evaporated to give 6 g of crude oily product. TLC (n-hexane/ethylacetate 9/0.2) indicated the presence of the mixture of title compounds; GC-MS: 325 [M+1]⁺.

### Intermediate 12: 8-Fluoro-11,11-di-2-bropen-1-yldibenzo[b,f]thiepin-10(11H)-one

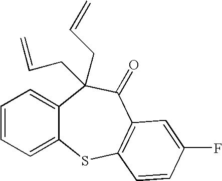

A mixture of 2-fluoro-10-(2-propen-1-yl)-11-(2-propen-1-yloxy)dibenzo[b,f]thiepin and 8-fluoro-11,11-di-2-propen-1-yldibenzo[b,f]thiepin-10(11H)-one (Intermediate 11, 5 g,15.38 mmol) was dissolved in o-xylene (35 mL) and the mixture was heated in a microwave reactor for 10 minutes at 250° C., 300W, 20 bar. After evaporation the solution yielded 4.9 g of a crude oil. TLC (n-hexane/ethylacetate=9/0.2) confirmed the presence of only one isomer. A sample (200 mg) of the crude oil was purified by silica gel column chromatography (n-hexane/ethylacetate=9/0.2) giving 65.8 mg of title compound; HPLC-MS: 325 [M+1]⁺; ¹HNMR (CDCl₃): δ 2.95-2.99 (q, 2H), 3.07-3.1 (q, 2H), 5.01-5.12 (m, 4H), 5.47-5.56 (m, 2H), 6.96-7.63 (m, 7H).

### Intermediate 13: 2′-Fluoro-11H-spiro[cyclopent-3-ene-1,10′-dibenzo[b,f]thiepin]-11′-one

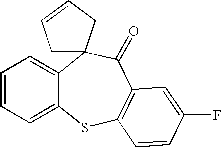

To a solution of 8-fluoro-11,11-di-2-propen-1-yldibenzo[b,f]thiepin-10(11H)-one (Intermediate 12, 2.7 g, 8.28 mmol) in degassed DCM (3 L) was added Hoveyda-Grubbs catalyst (2^{nd} generation, 13mol %, 0.425 g) under argon atmosphere at room temperature. The reaction mixture was stirred at room temperature for 5 hours and then filtered through a prepacked 10 g silica-gel cartridge (Supelco). The resulting filtrate was evaporated and purified by silica gel column chromatography (n-hexane/ethylacetate=9/0.2) to give 1.58 g of an oily compound. Crystallisation from n-hexane gave a white powder (450 mg); GC-MS: 297 [M+1]⁺; HPLC-UV: 99% purity; ¹HN MR (CDCl₃): δ 2.97-3.00 (d, 2H), 3.75-3.78 (d, 2H), 5.74 (s, 2H), 7.09-7.69 (m, 7H).

### Intermediate 14: 2′-Fluoro-3-hydroxy-11′H-spiro[cyclopentane-1,10′-dibenzo[f]thiepin]-11′-one

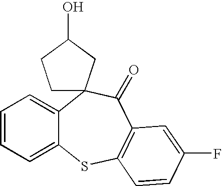

A 1M solution of BH₃-THF (8 mL, 8 mmol) was dropwise added to a stirred solution of 2′-fluoro-11′H-spiro[cyclopent-3-ene-1,10′-dibenzo[b,f]thiepin]-11′-one (Intermediate 13, 2.34 g, 7.4 mmol) in dry THF (100 mL) under argon atmosphere and stirred for 5 hours at room temperature. The mixture was then treated with water (20 mL) and 10% NaOH solution (10 mL) followed by addition of H₂O₂ (35% solution, 5 mL). Stirring was continued overnight at room temperature. The reaction mixture was diluted with 40 mL of water and the product was extracted with diethyl ether. The organic layer was then dried over anhydrous MgSO₄/Na₂SO₄ (1:5 ratio) and evaporated to give 2.1 g of the title compound which was used without further purification.

### Intermediate 15: 2′-Fluoro-3H,11′H-spiro[cyclopentane-1,10′-dibenzo[b,f]thiepin]-3,11′-dione

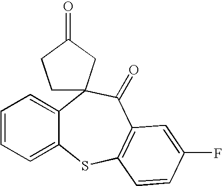

To a solution of 2′-fluoro-3-hydroxy-11′H-spiro[cyclopentane-1,10′-dibenzo[b,f]thiepin]-11′-one (Intermediate 14, 2.1 g, 6.68 mmol) in dry DCM was added Dess-Martin (triacetoxyperiodinane) (9 g, 21,31 mmol). The reaction mixture was stirred under argon at room temperature for 4 hours. The reaction was terminated by addition of 1M NaOH solution. The organic layer was separated and dried over anhydrous MgSO₄/Na₂SO₄ (1:5 ratio) and evaporated to give 1.9 g of crude compound. A sample of the crude (780 mg) compound was purified by silica gel column chromatography (n-hexane/ethylacetate=4/1) to give 510 mg of analytically pure compound; HPLC-MS m/z: 312.89 [M+1]⁺; GC-MS m/z: 313 [M+1]⁺; ¹HNMR (CDCl₃): δ 2.09-2.30 (m, 1H), 2.35-2.49 (m, 2H), 2.84-2.91 (d, 1H), 3.54-3.72 (m, 2H), 7.14-7.76 (m, 7H).

### Intermediate 16: Ethyl morpholine-2-carboxylate trifluoroacetate salt

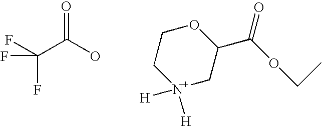

A suspension of ethyl 4-(phenylmethyl)-2-morpholinecarboxylate (prepared according to procedure described on J. of Med. Chem. 1993, vol. 36, No. 6, 683-689) (900 mg, 3.61 mmol), TFA (0.278 mL, 3.61 mmol), and Pd/C (150 mg, 1.410 mmol) in EtOH (15 ml), was degassed four times under nitrogen and then degassed three times under H₂. The reaction mixture was left stirring under H₂ at 25° C. for 4 h. MS monitor showed the reaction was complete. The reaction mixture was filtered and solvent was removed to give the title compound (950 mg);

m/z (ES): 160 [M+H]+; ¹H NMR (400 MHz, CHLOROFORM-d) d ppm 10.5(bs, 1H) 4.53-4.50 (m, 1H) 4.33-4.27 (q, 2H) 4.22-4.19 (m, 1H) 4.05-3.98 (m, 1H) 3.79-3.74 (m, 1H) 3.65-3.61 (m, 1H) 3.34-3.31 (m, 1H) 3.24-3.18 (m, 1H) 1.35-1.32 (t, 2H) 1.29-1.25 (t, 1H).

### Intermediate 17: 8-Chloro-11,11-di-2-bropen-1-yldibenzo[b,f]oxepin-10(11H)-one

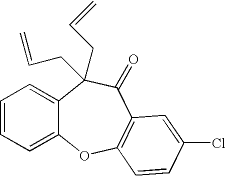

The solution of KOtBu (2.47 g,22 mmol) in 100 ml of the tBuOH was stirred for 10 min. under the stream of argon at room temperature. Then 8-chloro-dibenzo[b,f]oxepin-10(11H)-one (1 g, 4.1 mmol, for preparation see Journal of Medicinal Chemistry, (1980), 23(5), 494-501) and allyl bromide (9.31 ml,21.8 mmol) were added. The reaction was then heated at 60° C. for 3 hours. After cooling to room temperature, saturated solution of NaHCO₃ (150 ml) was added in one portion. The mixture was left under magnetic stirring for additional 15 minutes. The precipitate was filtered off and the aqueous phase was then extracted using ethyl-acetate (3×50 ml). Collected organic layers were washed with saturated NaHCO₃ solution, dried with anhydrous Na₂SO₄ and evaporated giving a crude oily product (1.8 g);

GC-MS m/z: 325 [M+1]⁺; ¹HNMR (CDCl3): δ 2.85-3.01 (m, 4H), 5.04-5.12 (m, 4H), 5.62-5.76 (m, 2H), 7.14-7.30 (m, 7H).

### Intermediate 18: 2′-Chloro-11H-spiro[cyclopent-3-ene-1,10′-dibenzo[b,f]oxepin]-11′-one

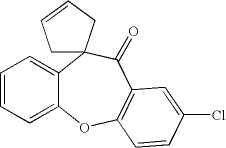

3 L of dry DCM were degassed with argon and 8-chloro-11,11-di-2-propen-1-yldibenzo[b,f]oxepin-10(11H)-one (Intermediate 17, 6.78 g, 20.8 mmol) and Hoveyda-Grubbs catalyst 2^{nd} generation (15.5 mol %, 1.275 g) were added. The solution was stirred under argon atmosphere for 8 h and then it was passed through a prepacked silica-gel column and evaporated. The crude oil (6.5 g) was purified by silica-gel column chromatography (n-Hexane:Ethyl-acetate=9:0.2) and fraction of the product that was obtained gave a precipitate after suspension in hexane (3.5 g);

GC-MS m/z: 297 [M+1]⁺; ¹HNMR (CDCl3): δ 2.92-3.95 (d, 2H), 3.42-3.46 (d, 2H), 5.72 (s, 2H), 7.20-7.35 (m, 6H), 7.45-7.47 (m, 1H).

### Intermediate 19: 2′-Chloro-3H,11′H-spiro[cyclopentane-1,10′-dibenzo[b,f]oxepin]-3,11′-dione

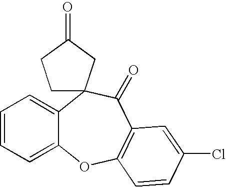

2′-Chloro-11′H-spiro[cyclopent-3-ene-1,10′-dibenzo[b,f]oxepin]-11′-one (Intermediate 18, 2.1 g,7.08 mmol) was melted in anhydrous THF (100m1) under argon atmosphere and 1M solution of BH₃-THF (8 ml, 8 mmol,) was added dropwise. The mixture was left for 4 h under stirring at room temperature. After 4 h H₂O (20 ml) and 10% NaOH (10 ml) were added followed by 30% H₂O₂ (5 ml).The mixture was stirred overnight and then diluted with H₂O (40 ml) and extracted with diethylether. Organic layer was washed with brine, dried and evaporated giving a crude flaky substance (approx. 2 g). The substance was dissolved in dry DCM (100 ml) and Dess-Martin (triacetoxyperiodinane) (9 g, 21,31 mmol) was added. The mixture was stirred overnight under argon atmosphere. The reaction was worked up by washing three times with NaOH (10% aqueous solution). Water layers were washed with DCM and combined organic layers were dried (Na₂SO₄/MgSO₄) and evaporated, to give a crude oil which under high vacuum turned to a flaky substance (2.13 g);

HPLC-MS m/z: 312.89 [M+1]+; Rt:8.44 min; GC-MS m/z: 313 [M+1]⁺;

1H NMR (CDCl3): δ 2.18-2.39 (m, 2H), 2.41-2.47 (m, 1H), 2.76-2.79 (d, 1H), 3.07-3.12 (m, 1H), 7.25-7.38 (m, 5H), 7.50-7.52 (m, 1H), 8.01-8.02 (d, 1H).

### Intermediate 20: 3H,11′H-spiro[cyclopentane-1,10′-dibenzo[b,f]oxepin]-3-one

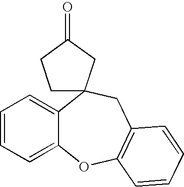

THF(10 ml) and glacial acetic acid (5 ml) were mixed in a Paar bottle and 2′-chloro-3H,11′H-spiro[cyclopentane-1,10′-dibenzo[b,f]oxepin]-3,11′-one (Intermediate 19, 100 mg, 0.319 mmol) and Pd/C (100 mg) were added. The mixture was shaken in the Paar apparatus under H₂ atmosphere (6 bar) at room temperature for 2 days. Then the mixture was filtered through a membrane filter and evaporated. The residue was dissolved in ethyl-acetate (10 ml) and washed with NaHCO₃(sat.) (3×15 ml). Organic layer was dried over Na₂SO₄/MgSO₄ and evaporated to give an oil (91.2 mg); GC-MS m/z: 265 [M+1]⁺; H NMR (CDCl3): δ 2.33-2.49 (m, 4H), 2.62-2.66 (d, 1H), 2.74-2.78 (d, 1H), 3.07-3.18 (m, 2H), 7.02-7.27 (m, 8H).

### Intermediate 21: 2-Methyl-1-(phenylmethyl)1,2-piperazinedicarboxylate

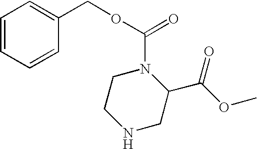

2-Methyl 1-(phenylmethyl) 1,2-piperazinedicarboxylate was prepared from the corresponding TFA salt (whose preparation is already known in literature eg. in Journal of Medicinal Chemistry (1990), 33(10), 2916-24 or Tetrahedron Letters (1989), 30(39), 5193-6.) To a DCM solution (5 ml) of (4-(1,1-dimethylethyl) 2-methyl 1-(phenylmethyl) 1,2,4-piperazinetricarboxylate (500 mg) was added, at 0° C., TFA (3m1) and the reaction temperature allowed to slowly reach 20° C. After complete conversion of the starting material DCM was evaporated, the crude was dissolved in water and extracted with Et₂O; then the water phase was basified (pH>9) with solid NaOH and extracted with DCM, the organic layer dried over Na₂SO₄ and the solvent evaporated to give a colourless oil (92 mg);

UPLC RT=0.47; m/z (ES): 279.1 [M−H]⁺; H NMR (400 MHz, CHLOROFORM-d) δ ppm 7.29-7.45 (m, 5H) 5.09-5.25 (m, 2H) 4.60-4.83 (m, 2H) 3.85-4.05 (m, 1H) 3.69-3.84 (m, 3H) 3.44-3.63 (m, 1H) 2.87-3.33 (m, 3H) 2.65-2.84 (m, 1H).

### Intermediate 22: Methyl 3-azetidinecarboxylate hydrochloride

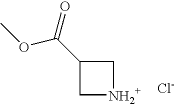

3-Azetidinecarboxylic acid (200 mg, 1.98 mmol) was suspended in anhydrous MeOH (5 mL) under argon atmosphere. Trimethylchlorosilane (500 μl, 3.91 mmol) was then added at room temperature and the mixture was stirred for 30 minutes and then left still overnight. The solvent was removed and the resulting solid was triturated in diethyl ether and decanted. The sample was dried under vacuum, to give the title compound as pale yellow solid (275 mg);

MS m/z (ES): 115.9 [M+H]⁺; H NMR (400 MHz, DMSO-d6) δ ppm 8.70-9.62 (m, 2H), 3.96-4.20 (m, 4H), 3.63-3.77 (m, 4H).

### Intermediate 23: Methyl 3-methyl-3-pyrrolidinecarboxylate HCl salt

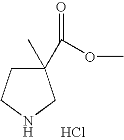

In a 50 mL round-bottomed flask 3-methyl-3-pyrrolidinecarboxylic acid (500 mg, 3.87 mmol, available from Tyger Scientific) and methanol (10 ml) were added. TMS-Cl (1 ml) was added and the reaction stirred at room temperature under nitrogen for 2 h. MS monitor showed that the reaction was not complete so TMS-Cl (484 μl) was added and the reaction stirred for another 2 h. MS monitor showed that the reaction was complete. The solvent was removed under reduced pressure and the crude dissolved in the minimum quantity of DCM and then Et₂O (about 40 ml) was added, a white precipitate formed which was decanted overnight. The excess of Et₂O was removed with a Pasteur and the solid dried under vacuum to give the title compound (670 mg, 3.36 mmol);

m/z (ES): 143.9 [M+H]⁺; H NMR (400 MHz, CHLOROFORM-d) d ppm 10.24-9.90 (bs, 1 H); 9.99-9.74 (bs, 1H); 3.79 (s, 3H); 3.63-3.35 (m, 2H); 3.25-3.08 (m, 1H); 2.59-2.38 (m, 1H); 2.10-1.88 (m, 1H); 1.48 (s, 3H).

### Intermediate 24: Methyl 4-amino-2,2-dimethylbutanoate

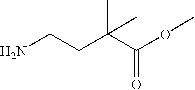

In a 50 mL round bottom flask was 4-amino-2,2-dimethylbutanoic acid HCl salt (500 mg, 2.98 mmol, available from Tyger Scientific) in methanol (10 ml). TMS-Cl (1.525 ml, 11.93 mmol) was added. The reaction was stirred at room temperature under nitrogen for 36 hrs and the MS monitor showed that the reaction was complete. Solvents were removed to give the crude title compound as a white solid (410 mg, 2.257 mmol); m/z (ES): 145.9 [M+H]⁺; ¹ NMR (400 MHz, CHLOROFORM-d) d ppm 3.72 (s, 3H); 2.97-2.93 (m, 2H); 1.91-1.87 (m, 2H); 1.26 (s, 6H).

### Intermediate 25: 3-Ethyl 1-(phenylmethyl) 1,3-piperidinedicarboxylate

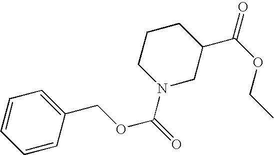

To a solution of ethyl 3-piperidinecarboxylate (1.976 ml, 12.72 mmol) in DCM (30 ml) was added TEA (2.66 ml, 19.08 mmol), then the mixture was cooled at 0° and benzyl chloroformate (1.998 ml, 13.99 mmol) was added slowly. The ice bath was removed and the reaction mixture was stirred at room temperature for 1.5 h. Then TEA (1.5 eq., 2.66 ml, 19.08 mmol) and benzyl chloroformate (0.5eq.) were added. The mixture was stirred for 3.5 hr and then was quenched and washed with water and diluted with DCM. The organic phase was washed with water followed by NaHCO₃. Evaporation of organic phase gave the crude which was purified on SiO₂ using cyclohexane/EtOAc 95:5 to 90:10. Evaporation of solvent gave the title compound (500 mg).

m/z (ES): 292.0[M+H]⁺

¹H NMR (400 MHz, CHLOROFORM-d) d ppm 7.5-7.15 (m, 5H); 5.2-5.0 (m, 2H); 4.40-3.75 (m, 4H); 3.0-2.8 (m, 1H); 2.6-2.4 (m, 1H); 2.15-1.90 (m, 1H); 2.85-1.35 (m, 4H); 1.30-1.15 (m, 3H)

### Intermediate 26: 3-Ethyl-1-(phenylmethyl) 3-fluoro-1,3-piperidinedicarboxylate

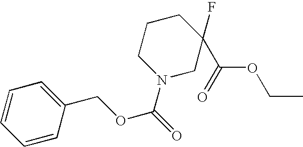

3-Ethyl 1-(phenylmethyl)1,3-piperidinedicarboxylate (Intermediate 25, 500 mg, 1.716 mmol) was dissolved in dry THF (10 ml) under nitrogen and the solution was cooled down to −78° C. Then LiHMDS (2.57 ml, 2.57 mmol) was added slowly and the reaction mixture was stirred while the temperature was gradually raised from −78° C. to 0° C. during 2 hrs. Then the reaction was cooled again to −40° C. and N-fluorobenzenesulfonimide (1082 mg, 3.43 mmol) dissolved in THF (4 ml) was added. The temperature was then gradually raised to room temperature over 5 hrs. The reaction was quenched with NH₄Cl sat. sol. and extracted with ethyl acetate, the combined organic layers were then dried over a phase separator and concentrated in vacuo. The crude product was purified by flash chromatography on silica 40M catridge, eluent cyclohexane/EtOAc 8:2. Evaporation of solvent gave a mixture containing the desired product. Further purification through Fraction Lynx gave a mixture of two enantiomers.

UPLC/MS RT=0.77; m/z (ES): 310.33 [M+H]+

This enantiomeric mixture was submitted for chiral HPLC separation (Preparative chromatographic conditions: Column: Chiralcel OJ-H, Mobile phase: n-Hexane/2-propanol 85/15% v/v, Flow rate: 1 mL/min; UV: 220 nm), to give two enantiomers:

### Intermediate 27: 3-Ethyl-1-(phenylmethyl) 3-fluoro-1,3-piperidinedicarboxylate (enantiomer 1)

retention time=13.49 mins (90 mg, 0.262 mmol); QC retention time=14.2 mins (Preparative chromatographic conditions: Column: Chiralcel OJ-H, Mobile phase: n-Hexane/2-propanol 85/15% v/v, Flow rate: 1 mL/min; UV: 220 nm)

¹H NMR (400 MHz, CHLOROFORM-d) d ppm 7.41-7.30 (m, 5H); 7.24-7.09 (m, 2H); 4.44-4.06 (m, 4H); 3.49-3.22 (m, 1H); 3.06-2.84 (m, 1H); 2.21-1.77 (m, 3H); 1.71-1.60 (m, 1H); 1.37-1.25 (m, 3H).

### Intermediate 28: 3-Ethyl-1-(phenylmethyl) 3-fluoro-1,3-piperidinedicarboxylate (enantiomer 2)

retention time=17.9 mins (93.4 mg, 0.272 mol); QC retention time=18.12 mins (Preparative chromatographic conditions: Column: Chiralcel OJ-H, Mobile phase: n-Hexane/2-propanol 85/15% v/v, Flow rate: 1 mL/min; UV: 220 nm)

¹H NMR (400 MHz, CHLOROFORM-d) d ppm 7.41-7.30 (m, 5H); 7.24-7.09 (m, 2H); 4.44-4.06 (m, 4H); 3.49-3.22 (m, 1H); 3.06-2.84 (m, 1H); 2.21-1.77 (m, 3H); 1.71-1.60 (m, 1H); 1.37-1.25 (m, 3H).

### Intermediate 27A: Ethyl 3-fluoro-3-piperidinecarboxylate (enantiomer 1)

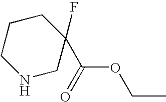

To a solution of 3-ethyl 1-(phenylmethyl) 3-fluoro-1,3-piperidinedicarboxylate (enantiomer 1, Intermediate 27, 90 mg, 0.294 mmol) in ethanol (7 ml), was added Pd/C (10%, 13 mg, 0.012 mmol) and the mixture was hydrogenated (1 atm) for 7 hours. The catalyst was removed from the reaction mixture by filtration and the crude solution was purified by SCX coloumn (5 g), to give ethyl 3-fluoro-3-piperidinecarboxylate (enantiomer 1, 15 mg).

¹H NMR (400 MHz, CHLOROFORM-d) d ppm 4.28 (q, 2H) 2.97-3.21 (m, 3H) 2.67-2.73 (m, 1H) 2.01-2.12 (m, 2H) 1.69-1.82 (m, 1H) 1.58-1.67 (m, 1H)

### Intermediate 29: Ethyl-3-methyl-3-piperidinecarboxylate

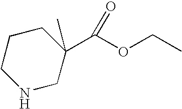

To a solution of ethyl 3-piperidinecarboxylate (0.988 ml, 6.36 mmol) in toluene (5 ml) at −35° C. was added NaHMDS (13.36 ml, 13.36 mmol) very slowly. During the addition the internal temperature was maintained under −20° C. Then the mixture was stirred between −25° C. and −20° C. for 30 min. Mel (0.398 ml, 6.36 mmol) was added portion wise maintaining the temperature between −25° C. and −20° C. Then the resulting mixture was stirred between −20° C. and −15° C. for 10 min, then warmed to room temperature and quenched with water (1 ml). Organic layer was separated and then washed with water two times. The organic layer was dried over phase separator. TFA (4.90 ml, 63.6 mmol) was added into the organic layer and left at room temperature for 1 hr. Then excess of TFA was removed under vacuum and further evaporation was done three times in presence of toluene. The crude was purified over SCX twice. Eluent: DCM followed by MeOH, and then with NH₃ in MeOH 2M. This purification gave the following desired products:

Trifluoroacetic salt: ¹H NMR (400 MHz, CHLOROFORM-d) d ppm 6.69-6.34 (bs, 2H); 4.27-3.97 (q, 2H); 3.50-3.29 (m, 1H); 3.16-3.03 (m, 1H); 2.84-2.66 (m, 1H); 2.66-2.51(m, 1H); 2.22-2.06 (m, 1H); 1.72-1.62 (m, 1H); 1.61-1.46 (m, 1H); 1.44-1.32 (m, 1H); 1.17-1.24 (t, 3H) 1.13 (s, 3H)

Free base: ¹H NMR (400 MHz, CHLOROFORM-d) d ppm 4.27-4.07 (m, 2H); 3.41-3.23 (m, 1H); 3.02-2.86 (m, 1H); 2.67-2.51 (m, 1H); 2.48-2.35 (m, 1H); 2.27-2.14 (m, 3H) 1.61-1.50 (m, 1H); 1.47-1.33 (m, 1H); 1.30-1.24 (m, 3H); 1.08-1.16 (s, 3H)

### Intermediate 30: 8-Fluoro-11,11-di-2-bropen-1-v1-5,11-dihydro-10H-dibenzo[a,d]cyclohepten-10-one

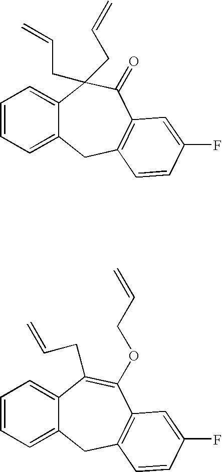

In a 100 mL round-bottomed flask potassium (0.950 g, 24.31 mmol) was added in tert-butanol (20.5 mL). The mixture was stirred at room temperature until complete dissolution of potassium to give a pale yellow solution. 8-fluoro-5,11-dihydro-10H-dibenzo[a,d]cyclohepten-10-one (2.2 g, 9.72 mmol, for preparation see Internation Patent Publication W02003/048146A1), previously dissolved in toluene (5 mL), and then allyl bromide (2.35 mL, 27.2 mmol) was added and the mixture heated at 60° C. for 2 hrs. The mixture was then cooled at room temperature and quenched with saturated aqueous NH₄Cl solution. The mixture was stirred for 15 mins then diluted with ethyl acetate, the organic phase separated and washed with brine and then dried over Na₂SO₄. After solvent evaporation the crude was purified by Biotage Si (40M) cartridge eluting with cHex. After solvent evaporation, the product was obtained was a colourless oil mixture of C-di-allyl compound and O/C-di-allyl compound (1.54 g).

UPLC/MS C-di-allyl compound RT=0.97; m/z (ES): 307.1 [M+H]⁺

¹H NMR (400 MHz, CHLOROFORM-d) ppm 7.30-7.40 (m, 7H) 5.58-5.46 (m, 2H) 5.09-4.96 (m, 4H) 3.59 (s, 2H) 2.99-2.77 (m, 4H);

O/C-di-allyl compound UPLC/MS RT=1.04; m/z (ES): 307.1 [M+H]⁺

¹H NMR (400 MHz, CHLOROFORM-d) ppm 7.30-7.40 (m, 7H) 6.08-5.93 (m, 2H) 5.30-5.15 (m, 4H) 4.45-4.30 (m, 2H) 4.10-4.03 (m, 3H) 3.53-3.46 (m, 1H).

### Intermediate 31: 8-Fluoro-11,11-di-2-propen-1-yl-5,11-dihydro-10H-dibenzo[a,d]cyclohepten-10-one

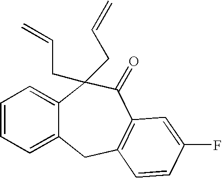

8-Fluoro-11,11-di-2-propen-1-yl-5,11-dihydro-10H-dibenzo[a,d]cyclohepten-10-one (Intermediate 30, 1.5 g, 4.9 mmol) was dissolved in toluene (10 mL×2) in a microwave vial and submitted to 30 min of MW irradiation at 200° C. Solvent was removed under vacuum and the crude purified by Biotage Si (40M) cartridge eluting with cHex/diethylether 99/1 v/v. After solvent evaporation, the title compound was obtained as yellowish oil (1.386 g).

UPLC/MS RT=0.97; m/z (ES): 307.1 [M+H]⁺

¹H NMR (400 MHz, CHLOROFORM-d) ppm 7.30-7.40 (m, 7H) 5.58-5.46 (m, 2H) 5.09-4.96 (m, 4H) 3.59 (s, 2H) 2.99-2.77 (m, 4H).

### Intermediate 32: 2′-Fluorospiro[cyclopent-3-ene-1,10′-dibenzo[a,d]cyclohepten]-11′(5′H)-one

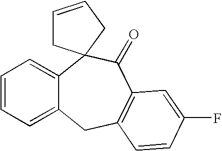

In a 1000 mL round-bottomed flask was added 8-fluoro-11,11-di-2-propen-1-yl-5,11-dihydro-10H-dibenzo[a,d]cyclohepten-10-one (Intermediate 31, 1.386 g, 4.52 mmol) in dry DCM (348 mL) to give a yellow solution. Grubbs II catalyst (576 mg, 0.679 mmol) was added and the solution stirred at room temperature for 4 hrs. The solvent was removed under vacuum and the crude was purified by Biotage Si (40M) cartridge eluting with cHex/diethylether 99/1 v/v to obtain the title compound as yellowish oil (1.11 g).

UPLC/MS RT=0.92; m/z (ES): 279.09 [M+H]⁺

### Intermediate 33: 2′-Fluoro-5′,11′-dihydrospiro[cyclopentane-1,10′-dibenzo[a,d]cycloheptene]-3,11′-diol

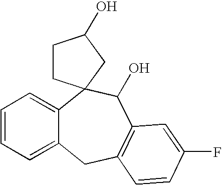

In a 100 mL round-bottomed flask 2′-fluorospiro[cyclopent-3-ene-1,10′-dibenzo[a,d]cyclohepten]-11′(5′H)-one (Intermediate 32, 1.3 g, 4.67 mmol) was dissolved in THF (16 mL) to give a yellow solution. Borane tetrahydrofuran complex (4.67 mL, 4.67 mmol) was added dropwise at 20° C. After 4 hours sodium hydroxide, 3 M solution, (0.62 mL) was added, followed by slow addition of hydrogen peroxide, 30% w/w solution (716 μL, 7.01 mmol). Mixture was then stirred for 16 hours at room temperature. Diethyl ether (15 mL) was added to the reaction mixture was washed with brine and water. The combined organic layers were dried over Na₂SO₄ and then the solvent removed under vacuum to obtain a crude yellowish foam product. The crude was purified by Biotage Si (25M) cartridge eluting with cHex/EtOAc gradient from 100/0 to 60/40 in 10 CV). The title compound was isolated as white foam (634 mg).

UPLC/MS RT=0.73; m/z (ES): 281.11 [M+H−18]⁺

### Intermediate 34: 2′-Fluoro-5′,11′-dihydrospiro[cyclopentane-1,10′-dibenzo[a,d]cyclohepten]-3-ol

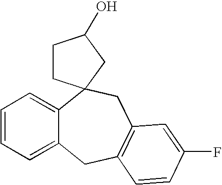

A solution of 2′-fluoro-5′,11′-dihydrospiro[cyclopentane-1,10′-dibenzo[a,d]cycloheptene]-3,11′-diol (Intermediate 33, 432 mg, 1.448 mmol) in THF/AcOH (4/1) (20 mL), was processed in the H-cube apparatus under H₂ atmosphere (30 atm) at 60° C. for 8 h. Solvent was removed under vacuum and the crude product was purified by flash chromatography Biotage Si (25M) cartridge eluting with cHex/EtOAc gradient from 100/0 to 70/30. The title compound was obtained as yellowish foam (200 mg).

UPLC/MS RT=0.80; m/z (ES): 265.13 [M+H−18]⁺. ¹H NMR (400 MHz, CHLOROFORM-d) ppm 7.24-6.77 (m, 7H) 5.32 (s, 1H) 4.77-4.62 (m, 1H) 4.20-4.03 (m, 2H), 3.35-3.31 (m, 1H), 3.12-2.95 (dd, 1H), 2.4-1.9 (m, 6H).

### Intermediate 35: 2′-Fluoro-5′,11′-dihydro-3H-spiro[cyclopentane-1,10′-dibenzo[a,d]cyclohepten]-3-one

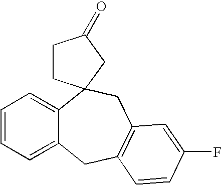

In a 50 mL round-bottomed flask was added Dess-MartinPeriodinane (841 mg, 1.983 mmol) in DCM (9.9 mL) to give a white suspension. 2′-fluoro-5′,11-dihydrospiro[cyclopentane-1,10′-dibenzo[a,d]cyclohepten]-3-ol (Intermediate 34, 280 mg, 0.992 mmol) in DCM (2 ml) was added and the slurry left under stirring at 20° C. for 2 h. The reaction was diluted with DCM (4 mL) and washed with NaOH (1N) and brine. The organic layer was dried over Na₂SO₄ and after solvent evaporation gave a crude product (290 mg) which submitted to chiral HPLC purification (Preparative chromatographic conditions: Column=Chiralcel OJ-H; n-Hexane/Ethanol/methanol (50/50) 65/35% v/v; Flow rate=0.8 ml/min; DAD=210-340 nm; CD=220 nm) to give two enantiomers:

### Intermediate 36: 2′-Fluoro-5′,11-dihydro-3H-spiro[cyclopentane-1,10′-dibenzo[a,d]cyclohepten]-3-one (enantiomer 1)

retention time 15.7 min (70 mg), UPLC/MS RT=1.21; m/z (ES): 281 [M+H]⁺. ¹H NMR (400 MHz, CHLOROFORM-d) ppm 7.60-6.80 (m, 7H) 4.10-4.30 (dd, 2H) 3.20-3.05 (dd, 2H) 2.75-2.49 (m, 4H), 2.40-2.25 (m, 2H).

### Intermediate 37: 2′-Fluoro-5′,11-dihydro-3H-spiro[cyclopentane-1,10′-dibenzo[a,d]cyclohepten]-3-one (enantiomer 2)

retention time 22.1 min (70 mg). UPLC/MS RT=1.21; m/z (ES): 281 [M+H]⁺. ¹H NMR (400 MHz, CHLOROFORM-d) ppm 7.60-6.80 (m, 7H) 4.10-4.30 (dd, 2H) 3.20-3.05 (dd, 2H) 2.75-2.49 (m, 4H), 2.40-2.25 (m, 2H).

### Intermediate 38: 2-Methyl 1-(phenylmethyl) 4-(5′,11¹-dihydrospiro[cyclopentane-1,10′-dibenzo[a,d]cyclohepten]-3-yl)-1,2-piperazinedicarboxylate

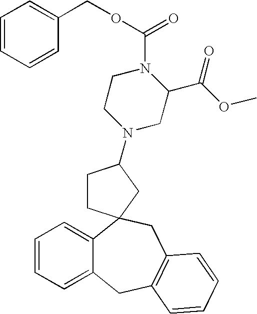

To a solution of (+) 5′,11′-dihydro-3H-spiro[cyclopentane-1,10′-dibenzo[a,d]cyclohepten]-3-one (Intermediate 7, 60 mg, 0.229 mmol) and 2-methyl 1-(phenylmethyl) 1,2-piperazinedicarboxylate (Intermediate 21, 76 mg, 0.274 mmol) in DCE (4 ml) under nitrogen was added AcOH (0.026 ml, 0.457 mmol). The reaction was stirred at room temperature for 1 h and then NaBH(OAC)₃ (72.7 mg, 0.343 mmol) was added and the resulting mixture was stirred overnight. The mixture was diluted with DCM. Organic phase was washed with NaHCO₃ sat. sol., brine and concentrated under vacuum. The crude mixture was purified through SiO₂ (redisep Catridge 12 g) using cyclohexane:EtOAc (From 100:00 to 80:20 for 25 min and 80:20 for 40 min) to afford title compound (106 mg, 0.202 mmol) as a mixture of two diastereoisomeric racemates. For the major diastereoisomer: ¹H NMR (400 MHz, CHLOROFORM-d) d ppm 7.43-6.98 (m, 8H); 5.26-5.13 (m, 2H); 4.61-4.88 (m, 1H); 4.24-4.01 (m, 2H); 3.84-3.62 (m, 3H); 3.48-2.74 (m, 7H); 2.32-1.75 (m, 10H); 1.45 (s, 3H).

### Intermediate 39: Methyl 4-(5′,11′-dihydrospiro[cyclopentane-1,10′-dibenzo[a,d]cyclohepten]-3-ylamino)-2,2-dimethylbutanoate

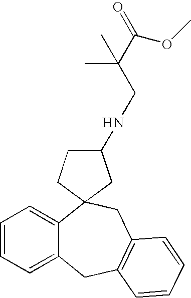

In a 50 mL round-bottomed flask was added 5′,11-dihydro-3H-spiro[cyclopentane-1,10′-dibenzo[a,d]cyclohepten]-3-one (Intermediate 5, 250 mg, 0.953 mmol), methyl 4-amino-2,2-dimethylbutanoate (Intermediate 24, 260 mg, 1.429 mmol), DIPEA (0.183 ml, 1.048 mmol) and AcOH (0.273 ml, 4.76 mmol) in DCM (5 ml) to give a colorless solution. The solution was stirred at room temperature for 1 hr and then NaBH(OAC)₃ (303 mg, 1.429 mmol) was added. The reaction mixture was stirred at room temperature overnight. MS monitor showed that the reaction was complete. NaHCO₃ was added, the phases separated and the organic washed with water. The aqueous was extracted with DCM and the phases separated on a phase separator cartridge. The combined organic extracts were evaporated to give the crude product (556 mg, 1.420 mmol) as a mixture of two diastereoisomeric racemates.

UPLC/MS RT=0.67; m/z (ES): 392.12 [M+H]⁺

For major isomers ¹H NMR (400 MHz, CHLOROFORM-d) d ppm 7.35-7.0 (m, 8H); 4.33-3.96 (m, 3H); 3.90-3.70 (m, 1H); 3.68 (s, 2H); 3.34-2.79 (m, 3H); 2.48-1.77 (m, 10 H); 1.22-1.20 (s, 6H).

### Intermediate 40: Methyl 4-[5′,11′-dihydrosbirorcyclopentane-1,10′-dibenzo[a,d]cyclohepten]-3-yl(methyl)amino]-2,2-dimethylbutanoate

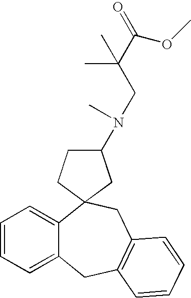

In a 50 mL round-bottomed flask was added methyl 4-(5′,11′-dihydrospiro[cyclopentane-1,10′-dibenzo[a,d]cyclohepten]-3-ylamino)-2,2-dimethylbutanoate (Intermediate 39, 556 mg, 1.420 mmol) and formaldehyde 37% water solution (0.211 ml, 2.84 mmol) in DCM (5 ml) to give a colorless solution. NaBH(OAC)₃ (451 mg, 2.130 mmol) was added and the solution stirred at room temperature overnight. MS monitor showed reaction was complete. NaHCO₃ was added, the phases separated and the organic washed with water. The aqueous was extracted with DCM. The phases separated on a phase separator cartridge. The combined organic extracts evaporated to give the crude product (453 mg, 1.117 mmol) as a mixture of two diastereoisomeric racemates.

UPLC/MS RT=0.68; m/z (ES): 406.14 [M+H]⁺

The isomeric mixture was submitted for chiral HPLC purification (Preparative chromatographic conditions: Column=Chiralcel OD-H; Mobile phase=n-Hexane/Ethanol 95/5% v/v; Flow rate=14 mL/min; DAD=225 nm, CD=225 nm) to give 1 single isomer (Intermediate 41) plus a mixture of the other 3 isomers

### Intermediate 41: Methyl 445′,11′-dihydrospiro[cyclopentane-1,10′-dibenzo[a,d]cyclohepten]-3-yl(methyl)amino]-2,2-dimethylbutanoate (isomer 4) retention time =6.52 mins (24 mg)

¹H NMR (400 MHz, CHLOROFORM-d) d ppm 7.51-7.39 (m, 1H); 7.27-6.97 (m, 7H); 4.3-3.9 (m, 2H); 3.67 (s, 3H); 3.21-3.04 (m, 3H); 2.5-1.75 (m, 13H);

The mixture of the 3 isomers retention time=5.55-5.90 mins 254 mg) was submitted to further chiral HPLC purification (Preparative chromatographic conditions: Column=Chiralcel OJ-H; Mobile phase=n-Hexane/Ethanol 80/20% v/v; Flow rate=14 mL/min; DAD=225 nm, CD=225 nm) to give 1 single isomer (isomer 3) not characterized, plus the diastereoisomer 1 as racemate (Intermediate 42):

### Intermediate 42: Methyl 4-[5′,11′-dihydrospiro[cyclopentane-1,10′-dibenzo[a,d]cyclohepten]-3-yl(methyl)amino]-2,2-dimethylbutanoate (diastereoisomer 1):

retention time=6.15 mins (208 mg)

¹H NMR (400 MHz, CHLOROFORM-d) d ppm 7.41-6.94 (m, 8H); 4.31-4.18 (m, 2H); 3.67 (s, 3H); 3.36-3.0 (m, 3H); 2.58-1.57 (m, 13H); 1.22 (s, 6H).

### Intermediate 43: Methyl [1-(5′,11′-dihydrospiro[cyclopentane-1,10′-dibenzo[a,d]cyclohepten]-3-yl)-3-pyrrolidinyl]acetate

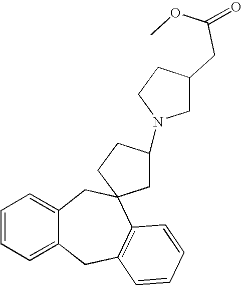

To a solution of (+) 5′,11′-dihydro-3H-spiro[cyclopentane-1,10′-dibenzo[a,d]cyclohepten]-3-one (Intermediate 7, 80 mg, 0.305 mmol) and methyl 3-pyrrolidinylacetate (65.5 mg, 0.457 mmol) in dry DCE (4 ml) under nitrogen, was added a drop of acetic acid and the mixture stirred at room temperature for 30 min. Sodium triacetoxyborohydride (97 mg, 0.457 mmol) was then added and the resulting reaction mixture was stirred for 3 h, quenched with NaHCO₃ (saturated aqueous solution) and extracted with DCM. The organic layers were combined, dried (Na₂SO₄) and concentrated in vacuo. The crude product was purified by flash chromatography on silica gel (25 g) eluting with a gradient of MeOH in DCM (from 0 to 5%) affording a mixture of two diastereoisomeric racemates, of the title compound (94 mg, 0.241 mmol). The isomeric mixture was submitted for chiral HPLC purification (Preparative chromatographic conditions: Column: Whelk O (R,R); Mobile phase: n-Hexane/2-Propanol 97/3% v/v; Flow rate:1.0 mL/min; UV: 225 nm), to give:

### Intermediate 44: Methyl 11-(5′,11′-dihydrospiro[cyclopentane-1,10′-dibenzo[a,d]cyclohepten]-3-yl)-3-pyrrolidinyl]acetate (diastereomeric mixture 3)

(24 mg), retention time=22.2 mins

¹H NMR (400 MHz, CHLOROFORM-d) d ppm 7.02-7.33 (m, 8H) 4.25 (d, 1H) 4.03 (d, 1 H) 3.70 (s, 3H) 3.30 (d, 1H) 3.15 (d, 1H) 2.85-3.05 (m, 2H) 2.40-2.75 (m, 5H) 1.80-2.25 (m, 8H) 2.97 1.40-1.55 (m, 1H).

### Intermediate 45: Methyl [1-(5′,11′-dihydrospiro[cyclopentane-1,10′-dibenzo[a,d]cyclohepten]-3-yl)-3-pyrrolidinyl]acetate (isomer 2)

(28 mg), retention time=24.1 mins

¹H NMR (400 MHz, CHLOROFORM-d) d ppm 7.02-7.33 (m, 8H) 4.25 (d, 1H) 4.03 (d, 1H) 3.70 (s, 3H) 3.30 (d, 1H) 3.15 (d, 1H) 2.85-3.05 (m, 2H) 2.40-2.75 (m, 5H) 1.80-2.25 (m, 8H) 2.97 1.40-1.55 (m, 1H).

### Compound 1: N-(5′,11-Dihydrospiro[cyclopentane-1,10′-dibenzo[,d]cyclohepten]-3-yl)-b-alanine formate salt (diastereomeric mixture 1)

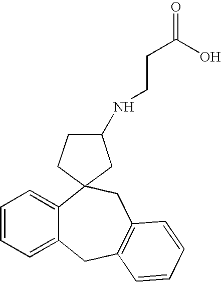

To a solution of methyl N-(5′,11′-dihydrospiro[cyclopentane-1,10′-dibenzo[a,d]cyclohepten]-3-yl)-β-alaninate (diastereomeric mixture 1, Intermediate 8, 21 mg, 0.060 mmol) in methanol/water (1.2/0.8, 2 mL) was added LiOH (7.20 mg, 0.300 mmol). The reaction was left to stir at room temperature for 3 h then the organic solvent was evaporated under vacuum. The aqueous phase was washed with DCM and then slowly acidified with 1N HCl (until pH 1). The resulting precipitate, was filtered and dried under vacuum to give 17 mg of a white solid. The solid was purified by Fraction Lynx (acid method). Solvent evaporation and trituration with Et₂O gave the title compound (8 mg) as mixture of diastereoisomers.

m/z (ES): 336.1 [M+H]⁺;

¹H NMR (400 MHz, DMSO-d₆) d ppm 8.14-8.28 (m, 1H) 6.90-7.33 (m, 6H) 3.99-4.18 (m, 2H) 2.78-3.92 (m, 8H) 1.66-2.35 (m, 8H).

### Compound 2: N-(5′,11-Dihydrospiro[cyclopentane-1,10′-dibenzo[a,d]cyclohepten]-3-yl)-b-alanine hydrochloride salt (diastereomeric mixture 2)

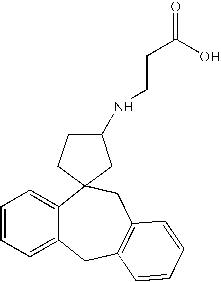

To a solution of methyl N-(5′,11′-dihydrospiro[cyclopentane-1,10′-dibenzo[a,d]cyclohepten]-3-yl-β-alaninate (diastereomeric mixture 2, Intermediate 9, 30 mg, 0.086 mmol) in methanol/water 3/2 (2 mL) was added LiOH (10.28 mg, 0.429 mmol) and the reaction mixture was left to stir for 1.5 h. The organic solvent was evaporated under vacuum, the resulting aqueous phase was washed with DCM and then slowly acidified with 3N HCl (until pH 1). The resulting precipitate was filtered and dried under vacuum to give 20 mg of a white solid, as mixture of diastereoisomers. Fraction Lynx/MS Rf=2.97; m/z (ES): 336.2 [M+H]⁺;

¹H NMR (500 MHz, DMSO-d₆) d ppm 12.71 (br. s., 1H) 8.99 (br. s., 1H) 6.96-7.62 (m, 8H) 3.85-4.25 (m, 3H) 2.92-3.52 (m, 5H) 2.59-2.83 (m, 2H) 1.73-2.32 (m, 6H).

### Compound 3: 1-(5′,11′-Dihydrospiro[cyclopentane-1,10′-dibenzo[a,d]cyclohepten]-3-yl)-1,2,5,6-tetrahydro-3-pyridinecarboxylic acid hydrochloride salt (diastereomeric mixture 1)

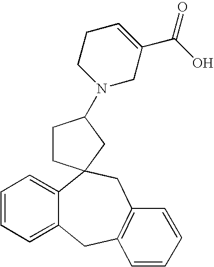

To a solution of methyl 1-(5′,11′-dihydrospiro[cyclopentane-1,10′-dibenzo[a,d]cyclohepten]-3-yl)-1,2,5,6-tetrahydro-3-pyridinecarboxylate (diastereomeric mixture 1, Compound 21, 55 mg, 0.142 mmol) in methanol/water 3/1 (2.4 mL) was added LiOH (16.99 mg, 0.710 mmol) and the reaction mixture was left to stir for 4 h. Further LiOH (17 mg, 0.71 mmol) was added and the reaction was left to stir overnight. The organic solvent was evaporated under vacuum and the aqueous phase washed with DCM. The reaction mixture was slowly acidified with 3N HCl checking the pH of the solution. A white precipitate formed at pH 1. The solid was filtered and dried under vacuum. The solid was triturated from EtOAc to give the title compound (41 mg); UPLC/MS Rf=0.62; m/z (ES): 374.3 [M+H]⁺; ¹H NMR (400 MHz, CHLOROFORM-d) d ppm 6.95-7.37 (m, 9H) 4.15-4.31 (m, 1H) 3.92-4.13 (m, 1H) 3.65-3.85 (m, 3H) 2.99-3.47 (m, 5H) 2.52-2.75 (m, 2H) 1.79-2.51 (m, 8H).

### Compound 4: N-(5′,11′-Dihydrospiro[cyclopentane-1,10′-dibenzo[a,d]cyclohepten]-3-yl)-N-methyl-β-alanine (diastereomeric mixture 1)

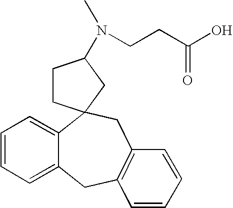

To a solution of N-methyl-5′,11′-dihydrospiro[cyclopentane-1,10′-dibenzo[a]cyclohepten]-3-amine (diastereomeric mixture 1, Compound 16, 48 mg) in methanol (2 mL) and water (1 mL) was added methyl 2-propenoate (0.031 mL, 0.346 mmol). The reaction mixture was heated at 100° C. for 30 min in a microwave reactor. LiOH (20.7 mg, 0.86 mmol) was then added and the reaction mixture was heated at 80° C. overnight. The solvents were then removed under vacuum and the crude product was purified by Fraction Lynx Preparative chromatographic conditions: Column: Gemini C18 AXIA, 50×21 mm, 5 μm; Mobile phase: A: NH₄HCO₃ sol. 10 mM, pH10; B: CH3CN; Gradient: 10% (B) for 1 min, 10% to 60% (B) in 9 min, 60% to 100% (B) in 0.5 min, 100% (B) for 2.5 min; Flow rate: 17 mL/min; UV range: 210-350 nm; Ionization: ES+; Mass range: 100-900 amu) to give the title compound (16 mg); HPLC/MS Rf=1.77; m/z (ES): 350.1 [M+H]⁺; ¹H NMR (400 MHz, DMSO-d₆) d ppm 7.08-7.41 (m, 7H) 6.96-7.06 (m, 1H) 3.95-4.25 (m, 2H) 2.99-3.47 (m, 4H) 2.62-2.79 (m, 2H) 2.31-2.42 (m, 2H) 2.20-2.28 (m, 3H) 1.67-2.08 (m, 5H).

### Compound 5: N-(5′,11′-Dihydrospiro[cyclopentane-1,10′-dibenzo[a,d]cyclohepten]-3-yl)-N-methyl-β-alanine (diastereomeric mixture 2)

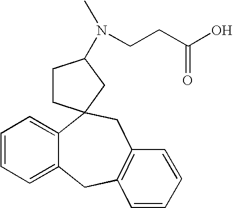

To a solution of N-methyl-5′,11′-dihydrospiro[cyclopentane-1,10′-dibenzo[a,d]cyclohepten]-3-amine (diastereomeric mixture 2, Compound 17, 48 mg) in methanol (2 mL) and water (1 mL) was added methyl 2-propenoate (0.031 mL, 0.346 mmol). The reaction mixture was heated at 100° C. for 30 min under microwave irradiation. LiOH (20.7 mg) was then added and the reaction mixture was heated at 80° C. overnight. Solvents were removed under vacuum to give a crude product that was dissolved in 3N HCl and was purified using an HLB cartridge eluting with MeOH to obtain 8 mg of the title product; HPLC/MS Rf=1.77; m/z (ES): 350.1 [M+H]⁺; ¹H NMR (400 MHz, DMSO-d₆) d ppm 6.96-7.40 (m, 8H) 3.95-4.27 (m, 2H) 2.95-3.36 (m, 4H) 2.65-2.78 (m, 2H) 2.33-2.44 (m, 2H) 2.22-2.33 (m, 3H) 1.64-2.09 (m, 5H).

### Compound 6: 4-[5′,11′-Dihydrospiro[cyclopentane-1,10′-dibenzo[a,d]cyclohepten]-3-yl(methyl)amino]butanoic acid (diastereomeric mixture 1)

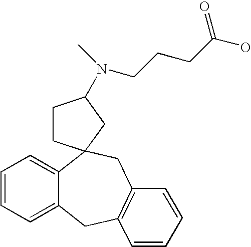

To a solution of methyl 4-[5′,11′-dihydrospiro[cyclopentane-1,10′-dibenzo[a,d]cyclohepten]-3-yl(methyl)amino]butanoate (diastereomeric mixture 1, Intermediate 10, 40 mg) in MeOH/H₂O (2 mL, 3/1 ratio), was added LiOH (5.07 mg, 0.212 mmol). The reaction mixture was stirred at room temperature for 2 days. The solvents were removed under vacuum and the crude product was dissolved in HCl (3N) and purified using an HLB cartridge (eluted with MeOH) to give the title product; UPLC/MS Rf=0.61; m/z (ES): 364.2 [M+H]⁺; ¹H NMR (400 MHz, DMSO-d₆) d ppm 7.24-7.25 (m, 8H) 3.98-4.25 (m, 2H) 3.01-3.49 (m, 5H) 2.51-2.61 (m, 4H) 2.25-2.36 (m, 3H) 1.61-2.08 (m, 6H).

### Compound 7: 1-(5′,11′-Dihydrospiro[cyclopentane-1,10′-dibenzo[a,d]cyclohepten]-3-yl)-1,2,5,6-tetrahydro-3-pyridinecarboxylic acid (isomer 2)

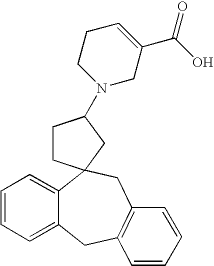

To a solution of methyl 1-(5′,11′-dihydrospiro[cyclopentane-1,10′-dibenzo[a,d]cyclohepten]-3-yl)-1,2,5,6-tetrahydro-3-pyridinecarboxylate (isomer 2, Compound 23, 70 mg, 0.181 mmol) in methanol (6 mL) and water (1 mL) was added LiOH (21.63 mg, 0.903 mmol). The reaction mixture was left to stir at room temperature overnight. Further LiOH (1.2 eq) and water (0.5 mL) were added and the resulting mixture was stirred at 40° for 6 hrs. The methanol was then evaporated under vacuum and the reaction mixture was acidified with 3N HCl (until pH˜1). This solution was purified using an HLB Cartridge (5 g) by eluting first with water and then MeOH, to give the title compound (56 mg); UPLC/MS Rf=0.61; m/z (ES): 374.2 [M+H]⁺; ¹H NMR (400 MHz, CHLOROFORM-d) d ppm 7.30-7.40 (m, 1H) 7.09-7.22 (m, 5H) 6.96-7.07 (m, 2H) 3.97-4.24 (m, 2H) 3.59-3.85 (m, 2H) 3.44-3.56 (m, 1H) 3.14-3.37 (m, 2H) 2.92-3.09 (m, 2H) 1.84-2.75 (m, 5H).

### Compound 8: 1-(5′,11′-Dihydrospiro[cyclopentane-1,10′-dibenzo[a,d]cyclohepten]-3-yl)-1,2,5,6-tetrahydro-3-pyridinecarboxylic acid (isomer 4)

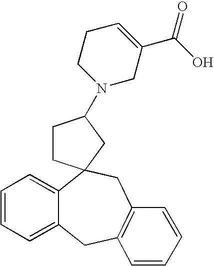

To a solution of methyl 1-(5′,11′-dihydrospiro[cyclopentane-1,10′-dibenzo[a,d]cyclohepten]-3-yl)-1,2,5,6-tetrahydro-3-pyridinecarboxylate (isomer 4, Compound 24; 11 mg, 0.028 mmol) in methanol (1.5 mL) and water (0.15 mL) was added LiOH (3.40 mg, 0.142 mmol). The reaction mixture was left to stir overnight at room temperature and then at 40° C. for 4 hrs. The methanol was evaporated under vacuum and the reaction mixture was acidified with 3N HCl checking the pH of the solution (until pH 1). This solution was purified using an HLB Cartridge (2 g) by eluting first with water and then MeOH to give the title compound (5.8 mg); UPLC/MS Rf=0.61; m/z (ES): 374.2 [M+H]⁺; ¹H NMR (400 MHz, CHLOROFORM-d) d ppm 7.47-7.73 (m, 1H) 6.86-7.33 (m, 8H) 3.98-4.28 (m, 2H) 3.60-3.96 (m, 3H) 2.85-3.36 (m, 4H) 1.86-2.74 (m, 7H) 1.10-1.39 (m, 2H).

### Compound 9: 1-(5′,11′-Dihydrospiro[cyclopentane-1,10′-dibenzo[a,d]cyclohepten]-3-yl)-4-piperidinecarboxylic acid (isomer 4)

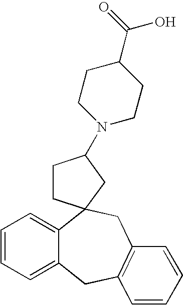

LiOH (0.593 mg, 0.025 mmol) was added to a solution of ethyl 1-(5′,11′-dihydrospiro[cyclopentane-1,10′-dibenzo[a,d]cyclohepten]-3-yl)-4-piperidinecarboxylate (isomer 4, Compound 27, 10 mg, 0.025 mmol) in ethanol (2 mL) and water (0.5 mL). The reaction mixture was left to stir at 70° C. for 4 hrs then at room temperature overnight. The mixture was stirred for a further 3 hrs at 70° C. The ethanol was evaporated under vacuum and the reaction mixture was acidified with 3N HCl until pH 1 was reached. The solution was purified using an HLB cartridge (1 g) by eluting first with water and then MeOH to give the title compound (3.8 mg); ¹H NMR (400 MHz, CHLOROFORM-d) d ppm 6.91-7.19 (m, 3H) 6.47-6.77 (m, 7H) 3.47-3.77 (m, 2H) 2.70-3.29 (m, 4H) 2.38-2.67 (m, 3H) 1.37-2.22 (m, 11H).

### Compound 10: (−)1-(5′,11′-Dihydrospiro[cyclopentane-1,10′-dibenzo[a,d]cyclohepten]-3-yl)-4-piperidinecarboxylic acid

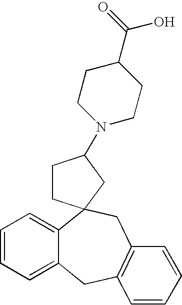

LiOH (17.80 mg, 0.743 mmol) was added to a solution of ethyl 1-(5′,11′-dihydrospiro[cyclopentane-1,10′-dibenzo[a,d]cyclohepten]-3-yl)-4-piperidinecarboxylate (isomer 2, Compound 26, 60 mg, 0.149 mmol) in ethanol (5 mL) and water (1 mL). The reaction mixture was left to stir at 70° C. for 4 hrs, overnight at room temperature and then at 70° C. for further for 3 hrs. The ethanol was evaporated under vacuum and the mixture was acidified with 3N HCl (until pH 1 was reached). The solution was purified using an HLB cartridge (6 g) by eluting first with water and then MeOH to afford the title compound (55 mg); [α]_{D}²⁰=−18.6° (c=0.63, MeOH) (optical rotation was measured on a different batch and for the HCl salt), UPLC/MS Rf=0.62; m/z (ES): 376.2 [M+H]⁺; ¹H NMR (400 MHz, CHLOROFORM-d) d ppm 7.30-7.44 (m, 1H) 6.99-7.23 (m, 7H) 3.95-4.25 (m, 2H) 3.14-3.71 (m, 6H) 1.80-2.70 (m, 12H).

### Compound 11: 1-(5′,11′-Dihydrospiro[cyclopentane-1,10′-dibenzo[a,d]cyclohepten]-3-yl)-3-piperidinecarboxylic acid (isomer 1)

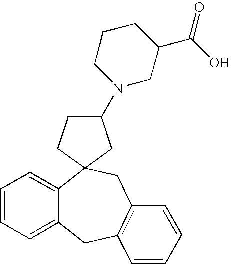

To a mixture of ethyl 1-(5′,11′-dihydrospiro[cyclopentane-1,10′-dibenzo[a,d]cyclohepten]-3-yl)-3-piperidinecarboxylate (isomer 1, Compound 31, 35 mg, 0.087 mmol) in ethanol (4.5 mL) and water (1 mL) was added KOH (19.46 mg, 0.347 mmol). The resulting mixture was stirred at 70° C. in a microwave reactor (Personal Chemistry Emrys™ Optimizer) for 30 min. The ethanol was evaporated under vacuum and the mixture acidified with 3N HCl (until pH 1 was reached). The solution was purified using an HLB cartridge (6 g) by eluting first with water and then MeOH to afford the title compound (32 mg); UPLC/MS Rf=0.61; m/z (ES): 376.2 [M+H]⁺; ¹H NMR (400 MHz, MeOD) d ppm 7.12-7.33 (m, 7H) 7.01-7.10 (m, 1H) 4.27 (d, 1H) 4.00-4.11 (m, 2H) 3.29-3.42 (m, 7H) 3.11-3.23 (m, 1H) 2.71-2.88 (m, 1H) 2.32-2.51 (m, 2H) 2.12-2.29 (m, 3H) 1.80-2.10 (m, 4H).

### Compound 12: 1-(5′,11′-Dihydrospiro[cyclopentane-1,10′-dibenzo[a,d]cyclohepten]-3-yl)-3-piperidinecarboxylic acid (isomer 2)

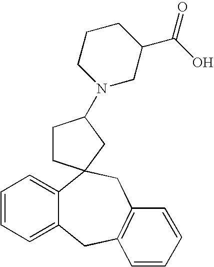

To a mixture of ethyl 1-(5′,11′-dihydrospiro[cyclopentane-1,10′-dibenzo[a,d]cyclohepten]-3-yl)-3-piperidinecarboxylate (isomer 2, Compound 32, 31 mg, 0.077 mmol) in ethanol (4.5 mL) and water (1 mL) was added KOH (21.55 mg, 0.384 mmol). The resulting mixture was stirred at 70° C. in a microwave reactor (Personal Chemistry Emrys™ Optimizer) for 30 min. The ethanol was evaporated under vacuum and the mixture acidified with 3N HCl (until pH 1 was reached). The solution was purified using an HLB cartridge (6 g) by eluting first with water and then MeOH to afford the title compound (22 mg); UPLC/MS Rf=0.64; m/z (ES): 376.2 [M+H]⁺; ¹H NMR (400 MHz, CHLOROFORM-d) d ppm 7.11-7.33 (m, 7H) 7.02-7.10 (m, 1H) 4.03-4.22 (m, 2H) 3.08-3.51 (m, 5H) 2.76-3.02 (m, 2H) 2.34-2.72 (m, 2H) 1.61-2.32 (m, 9H).

### Compound 13: 1-(2′-Fluoro-11′-oxo-11′H-spiro[cyclopentane-1,10′-dibenzo[b,f]thiepin]-3-yl-4-piperidinecarboxylic acid

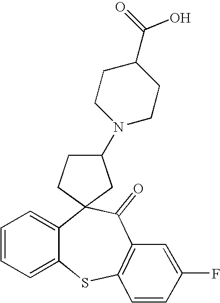

In a sealed vial, ethyl 1-(2′-fluoro-11′-oxo-11′H-spiro[cyclopentane-1,10′-dibenzo[b,f]thiepin]-3-yl)-4-piperidinecarboxylate (Compound 33 10 mg, 0.022 mmol) was heated at 80° C. for 3 hours in 3N HCl solution in water (2 mL). The water was evaporated and the solid triturated with cHex. The solvent was removed to afford the title compound as an off-white solid (8 mg); UPLC/MS Rf=0.62; m/z (ES): 426.1 [M+H]⁺; ¹H NMR (400 MHz, DMSO-d₆) d ppm 12.54 (br. s., 1H) 9.93-10.21 (m, 1H) 7.63-7.77 (m, 3H) 7.43-7.62 (m, 3H) 7.30-7.40 (m, 1H) 3.49-3.63 (m, 1H) 3.38-3.48 (m, 1H) 3.18-3.29 (m, 1H) 2.96-3.08 (m, 1H) 2.79-2.94 (m, 2H) 2.60-2.75 (m, 1H) 2.22-2.37 (m, 1H) 1.84-2.20 (m, 3H) 1.59-1.82 (m, 3H).

### Compound 14: 3-(5′,11′-Dihydrospiro[cyclopentane-1,10′-dibenzo[a,d]cyclohepten]-3-yl)-3-azabicyclo[3.1.0]hexane-1-carboxylic acid (isomer 1)

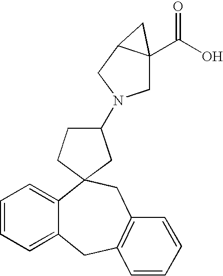

To a mixture of ethyl 3-(5′,11′-dihydrospiro[cyclopentane-1,10′-dibenzo[a,d]cyclohepten]-3-yl)-3-azabicyclo[3.1.0]hexane-1-carboxylate (isomer 1, Compound 48, 28 mg, 0.070 mmol,) in ethanol (5 mL) and water (1 mL) was added KOH (15.65 mg, 0.279 mmol). The resulting mixture was heated at 100° C. in a microwave reactor (Personal Chemistry Emrys™ Optimizer) for 30 min and then for 30×2 min at 100° C. Further KOH was added (1eg) and the mixture heated at 100° C. for 30 min. The solvent was evaporated under vacuum and the mixture acidified with 3N HCl (until pH 1 was reached). The solution was purified with a HLB cartridge (6 g) eluting first with water and then MeOH to afford the title compound (21.8 mg); UPLC/MS Rf=0.63; m/z (ES): 374.2 [M+H]⁺; ¹H NMR (400 MHz, CHLOROFORM-d) d ppm 6.96-7.22 (m, 8H) 3.97-4.21 (m, 2H) 3.02-3.70 (m, 8H) 1.99-2.51 (m, 7H) 1.77-1.97 (m, 1H).

### Compound 15: 3-(5′,11′-Dihydrospiro[cyclopentane-1,10′-dibenzo[a,d]cyclohepten]-3-yl)-3-azabicyclo[3.1.0]hexane-1-carboxylic acid (isomer 2)

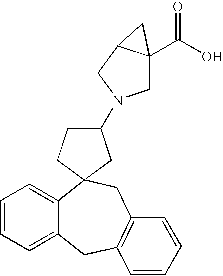

To a mixture of ethyl 3-(5′,11′-dihydrospiro[cyclopentane-1,10′-dibenzo[a,d]cyclohepten]-3-yl)-3-azabicyclo[3.1.0]hexane-1-carboxylate (isomer 2, Compound 49, 26 mg, 0.065 mmol,) in ethanol (5 mL) and water (1 mL) was added KOH (14.53 mg, 0.259 mmol). The resulting mixture was heated at 100° C. in a microwave reactor (Personal Chemistry Emrys™ Optimizer) for 30 min. The solvent was then evaporated under vacuum and the mixture acidified with 3N HCl (until pH 1 was reached). The solution was purified with a HLB cartridge (6 g) eluting first with water and then MeOH to afforded the title compound (18.8 mg); UPLC/MS Rf=0.63; m/z (ES): 374.2 [M+H]⁺; ¹H NMR (400 MHz, CHLOROFORM-d) d ppm 7.40-7.57 (m, 1H) 7.08-7.25 (m, 5H) 6.97-7.08 (m, 2H) 4.02-4.19 (m, 2H) 3.88-4.02 (m, 1H) 3.52-3.84 (m, 3H) 3.21-3.37 (m, 2H) 3.05-3.18 (m, 1H) 2.53-2.72 (m, 1H) 2.34-2.53 (m, 1H) 2.03-2.28 (m, 4H) 1.73-1.97 (m, 2H) 1.58-1.71 (m, 1H).

### Compound 16: N-methyl-5′,11′-dihydrospiro[cyclopentane-1,10′-dibenzo[a,d]cyclohepten]-3-amine (diastereomeric mixture 1)

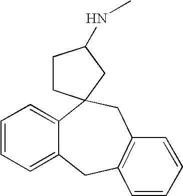

To a solution of (−)5′,11′-dihydro-3H-spiro[cyclopentane-1,10′-dibenzo[a,d]cyclohepten]-3-one (Intermediate 6, 140 mg, 0.53 mmol) in dichloromethane (5 mL) was added a 2M solution of methylamine in THF (0.320 mL). NaBH(OAC)₃ (170 mg, 0.800 mmol) was added and the reaction was left to stir overnight at room temperature. The mixture was washed with a saturated aqueous solution of NaHCO₃ and the organic phase was separated and evaporated under vacuum. The crude was purified using a SCX cartridge to obtain 148 mg of the title compound as mixture of diastereoisomers; UPLC/MS Rf=0.56; m/z (ES): 278.1 [M+H]⁺; ¹H NMR (400 MHz, CHLOROFORM-d) d ppm 6.92-7.56 (m, 8H) 3.99-4.31 (m, 2H) 3.34-3.54 (m, 1H) 3.05-3.32 (m, 2H) 2.43-2.55 (m, 3H) 1.65-2.34 (m, 6H).

### Compound 17: N-methyl-5′,11′-dihydrospiro[cyclopentane-1,10′-dibenzo[a,d]cyclohepten]-3-amine (diastereomeric mixture 2)

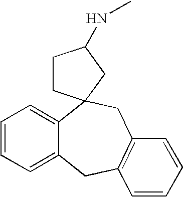

To a solution of (+)5′,11′-dihydro-3H-spiro[cyclopentane-1,10′-dibenzo[a,d]cyclohepten]-3-one (Intermediate 7, 140 mg, 0.53 mmol) in dichloromethane (5 mL) was added 2M solution of methylamine in THF (0.32 mL). NaBH(OAC)₃ (170 mg, 0.800 mmol) was added portion-wise and the reaction was left overnight at room temperature. The mixture was then washed with a saturated aqueous solution of NaHCO₃ and the organic phase was separated and evaporated under vacuum. The crude mixture was purified using a SCX cartridge to obtained 146 mg of the title compound as a mixture of diastereoisomers; UPLC/MS Rf=0.58; m/z (ES): 278.1 [M+H]⁺; ¹H NMR (400 MHz, CHLOROFORM-d) d ppm 6.92-7.56 (m, 8H), 3.99-4.31 (m, 2H), 3.34-3.54 (m, 1H), 3.05-3.32 (m, 2H), 2.43-2.55 (m, 3H), 1.65-2.34 (m, 6H).

### Compound 18: 1-(5′,11′-Dihydrospiro[cyclopentane-1,10′-dibenzo[a,d]cyclohepten]-3-yl)-4-fluoro-4-piperidinecarboxylic acid (isomer 2)

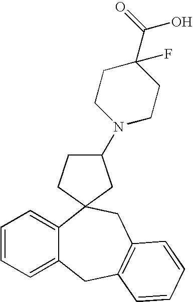

To a solution of ethyl 1-(5′,11′-dihydrospiro[cyclopentane-1,10′-dibenzo[a,d]cyclohepten]-3-yl)-4-fluoro-4-piperidinecarboxylate (isomer 2, Compound 36) (21 mg, 0.050 mmol) in THF (1.5 ml) and water (0.5 ml), was added LiOH (4.77 mg, 0.199 mmol) and the mixture was heated under reflux for 4 hours. The solvent was concentrated at reduced pressure, and the residue dissolved in water then neutralized with HCl (1M). This mixture was purified by C18 column (10 g) to give the title compound (14 mg) as a white solid. ¹H NMR (500 MHz, CHLOROFORM-d) d ppm 7.00-7.40 (m, 8H) 4.15 (d, 1H) 4.06 (d, 1H) 3.71-3.85 (m, 1H) 3.51-3.70 (m, 1H) 3.29-3.44 (m, 1H) 3.31 (d, 1H) 3.17 (d, 1H) 2.84-3.03 (m, 2H) 2.49-2.83 (m, 4H) 2.11-2.36 (m, 5H) 1.79-2.00 (m, 1H). UPLC/MS Rf=0.64; m/z (ES): 394.2 [M+H]⁺.

### Compound 19: 4-(5′,11′-Dihydrospiro[cyclopentane-1,10′-dibenzo[a,d]cyclohepten]-3-yl)-2-morpholinecarboxylic acid (isomer 1)

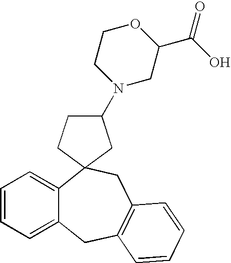

In a round-bottomed flask, a isomer of ethyl 4-(5′,11′-dihydrospiro[cyclopentane-1,10′-dibenzo[a,d]cyclohepten]-3-yl)-2-morpholinecarboxylate (isomer 1, Compound 41, 26 mg, 0.064 mmol) was dissolved in ethanol (2 ml) and water (0.5 ml). LiOH (7.68 mg, 0.321 mmol) was added and the reaction mixture was left to stir at room temperature overnight. The ethanol was evaporated under vacuum and the residue was acidified with 3N HCl checking the pH of the solution (until pH 1). The mixture was purified using an HLB Cartridge (5 g), eluting first with water, and secondly with MeOH. Evaporation of the MeOH afforded the title compound (21.2 mg, 0.050 mmol); UPLC Rt=0.60; m/z (ES): 378.22 [M+H]⁺; ¹H NMR (400 MHz, METHANOL-d) d ppm 7.69-6.92 (m, 8H), 4.31-3.42 (m, 8H), 4.28-2.87 (m, 3H) 2.86-1.95 (m, 7H).

### Compound 20: 4-(5′,11′-Dihydrospiro[cyclopentane-1,10′-dibenzo[a,d]cyclohepten]-3-yl)-2-morpholinecarboxylic acid (isomer 2)

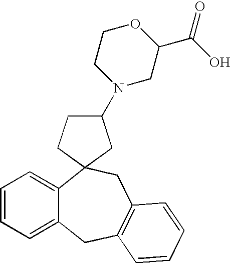

In a round-bottomed flask, a isomer of ethyl 4-(5′,11′-dihydrospiro[cyclopentane-1,10′-dibenzo[a,d]cyclohepten]-3-yl)-2-morpholinecarboxylate (isomer 2, Compound 44, 28 mg, 0.069 mmol) was dissolved in ethanol (3 ml) and water (1 ml). LiOH (8.27 mg, 0.345 mmol) was added, the reaction mixture was left under stirring at room temperature for 4 hours. The ethanol was evaporated under vacuum and the residue was acidified with 3N HCl checking the pH of the solution (until pH 1). The mixture was purified using an HLB Cartridge (5 g), eluting firstly with water, and secondly with MeOH. Evaporation of the MeOH afforded the title compound (25 mg, 0.059 mmol); UPLC Rt=0.61; m/z (ES): 378.15 [M+H]⁺; ¹H NMR (400 MHz, METHANOL-d) d ppm 7.36-7.03 (m, 8H), 4.51-3.74 (m, 8H), 3.52-3.4 (m, 1H) 3.27-3.15 (m, 2H) 2.5-1.92 (m, 7H).

### Compound 21: Methyl 1-(5′,11′-dihydrospiro[cyclopentane-1,10′-dibenzo[a,d]cyclohepten]-3-yl)-1,2,5,6-tetrahydro-3-pyridinecarboxylate (diastereomeric mixture 1)

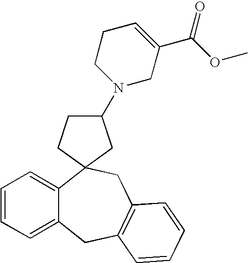

To a solution of (−)5′,11′-dihydro-3H-spiro[cyclopentane-1,10′-dibenzo[a,d]cyclohepten]-3-one, (Intermediate 6, 60 mg, 0.229 mmol) in 1,2 DCE (2 mL) were added methyl 1,2,5,6-tetrahydro-3-pyridinecarboxylate (40.6 mg, 0.229 mmol) and DIPEA (0.044 mL, 0.252 mmol). After 10 min stirring at room temperature, AcOH (0.026 mL, 0.457 mmol) and NaBH(OAc)₃ (72.7 mg, 0.343 mmol) were added. The reaction mixture was stirred at room temperature overnight. The mixture was washed with saturated aqueous NaHCO₃, solution then brine. The layers were separated and the organic layer was evaporated under vacuum to afford a yellow oil, which was dissolved in methanol and purified using a 1 g SCX cartridge (eluting firstly with MeOH then 2M NH₃ in MeOH). Evaporation of the solvent gave 55 mg of the title product as a mixture of diastereoisomers as yellow foam; UPLC/MS Rf=0.63; m/z (ES): 388.1 [M+H]⁺; ¹H NMR (400 MHz, CHLOROFORM-d) d ppm 6.95-7.37 (m, 9H) 4.15-4.31 (m, 1H) 3.92-4.13 (m, 1H) 3.65-3.85 (m, 3H) 2.99-3.47 (m, 5H) 2.52-2.75 (m, 2H) 1.79-2.51 (m, 8H).

### Compound 22: Methyl 1-(5′,11′-dihydrospiro[cyclopentane-1,10′-dibenzo[a,d]cyclohepten]-3-yl)-1,2,5,6-tetrahydro-3-pyridinecarboxylate (diastereomeric mixture 2)

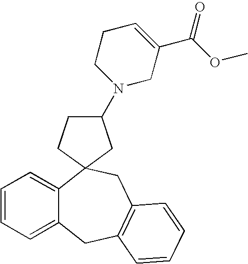

To a solution of (+)5′,11′-dihydro-3H-spiro[cyclopentane-1,10′-dibenzo[a,d]cyclohepten]-3-one, (Intermediate 7,100 mg, 0.38 mmol) in 1,2-dichloroethane (5 mL) were added methyl 1,2,5,6-tetrahydro-3-pyridinecarboxylate hydrochloride (67.7 mg, 0.381 mmol) and DIPEA (0.073 mL, 0.419 mmol). After 10 min stirring at room temperature, AcOH (0.044 mL, 0.762 mmol) and NaBH(OAC)₃ (121 mg, 0.572 mmol) were added and the reaction mixture was stirred at room temperature overnight. Further guvacine methyl ester hydrochloride (0.3 eq) and NaBH(OAC)₃ (0.5 eq) were added and the mixture was stirred for an additional 4 hrs. The reaction was washed with saturated aqueous NaHCO₃ solution, then brine and the organic phase was separated and concentrated under vacuum. The crude product was purified using a SCX cartridge eluting firstly with DCM, secondly MeOH and thirdly NH₃ in MeOH (2M). After solvent evaporation 94 mg of the desired compound, Compound 22, was obtained as mixture of diastereoisomers; UPLC/MS Rf=0.66; m/z (ES): 388.2 [M+H]⁺

The diastereomeric mixture 2 of methyl 1-(5′,11′-dihydrospiro[cyclopentane-1,10′-dibenzo[a,d]cyclohepten]-3-yl)-1,2,5,6-tetrahydro-3-pyridinecarboxylate (Compound 22) was submitted for chiral HPLC purification (Preparative chromatographic conditions: Column=Chiralcel OD-H; Mobile phase=n-Hexane/Ethanol 75/25% v/v; Flow rate=0.8 mL/min; DAD=210-340 nm; CD=225 nm) to give:

### Compound 23: Methyl 1-(5′,11′-dihydrospiro[cyclopentane-1,10′-dibenzo[a,d]cyclohepten]-3-yl)-1,2,5,6-tetrahydro-3-pyridinecarboxylate (isomer 2)

retention time=8 minutes (70 mg); ¹H NMR (400 MHz, CHLOROFORM-d) d ppm 6.98-7.37 (m, 9H) 4.27 (d, 2H) 4.02 (d, 2H) 3.72-3.79 (m, 3H) 3.36-3.45 (m, 1H) 3.21-3.34 (m, 2H) 3.05-3.19 (m, 2H) 2.55-2.73 (m, 2H) 2.34-2.50 (m, 2H) 2.20-2.33 (m, 2H) 2.08-2.19 (m, 1H) 1.80-2.05 (m, 3H).

### Compound 24: Methyl 1-(5′,11′-dihydrospiro[cyclopentane-1,10′-dibenzo[a,d]cyclohepten]-3-yl)-1,2,5,6-tetrahydro-3-pyridinecarboxylate (isomer 4)

retention time=10 minutes (11 mg); ¹H NMR (400 MHz, CHLOROFORM-d) d ppm 7.39-7.52 (m, 1H) 7.09-7.27 (m, 6H) 6.99-7.09 (m, 2H) 4.24 (d, 1H) 4.06 (d, 1H) 3.75 (s, 3H) 3.01-3.40 (m, 5H) 2.54-2.74 (m, 2H) 2.25-2.46 (m, 2H) 1.87-2.19 (m, 4H).

### Compound 25: Ethyl 1-(5′,11′-dihydrospiro[cyclopentane-1,10′-dibenzo[a,d]cyclohepten]-3-yl)-4-piperidinecarboxylate (diastereomeric mixture 2)

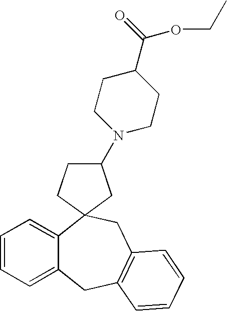

To a solution of (+)5′,11′-dihydro-3H-spiro[cyclopentane-1,10′-dibenzo[a,d]cyclohepten]-3-one (Intermediate 7, 74 mg, 0.27 mmol) and ethyl isonipecotate (0.082 mL, 0.534 mmol) in 1,2-dichloroethane (4 mL) was added AcOH (0.076 mL, 1.334 mmol). The reaction mixture was stirred at room temperature for 1 h and then NaBH(OAC)₃ (85 mg, 0.400 mmol) was added and the resulting mixture was stirred overnight. The mixture was diluted with DCM and washed with a saturated solution of NaHCO₃, brine. The phases were separated and the organic layer concentrated under vacuum. The crude mixture was purified by passing through a 12 g Si-redisep Cartridge 12 g (eluting with cHex:EtOAc; from 100:00 to 80:20) to afford the title compound (82 mg) as mixture of two diastereoisomers. UPLC/MS Rf=0.63; m/z (ES): 404.2 [M+H]⁺

This diastereomeric mixture of ethyl 1-(5′,11′-dihydrospiro[cyclopentane-1,10′-dibenzo[a,d]cyclohepten]-3-yl)-4-piperidinecarboxylate (Compound 25) was submitted for chiral chromatography purification (Preparative chromatographic conditions: Column=Chiralcel OD-H; Mobile phase=n-Hexane/Isopropanol 85/15% v/v; Flow rate=1 mL/min; DAD=210-340 nm; CD=225 nm) to give:

### Compound 26: Ethyl 1-(5′,11′-dihydrospiro[cyclopentane-1,10′-dibenzo[a,d]cyclohepten]-3-yl)-4-piperidinecarboxylate (isomer 2)

retention time=7 minutes (60 mg); ¹H NMR (400 MHz, CHLOROFORM-d) d ppm 6.84-7.48 (m, 8H) 3.86-4.40 (m, 3H) 2.74-3.44 (m, 4H) 1.62-2.48 (m, 15H) 1.16-1.39 (m, 3H).

### Compound 27: Ethyl 1-(5′,11′-dihydrospiro[cyclopentane-1,10′-dibenzo[a,d]cyclohepten]-3-yl-4-piperidinecarboxylate (isomer 4)

retention time=10 minutes (10 mg); ¹H NMR (400 MHz, CHLOROFORM-d) d ppm 7.40-7.52 (m, 1H) 6.97-7.33 (m, 7H) 3.91-4.32 (m, 6H) 3.43-3.56 (m, 1H) 2.78-3.30 (m, 4H) 1.68-2.42 (m, 11H) 1.14-1.36 (m, 3H).

### Compound 28: Ethyl 1-(5′,11′-dihydrospiro[cyclopentane-1,10′-dibenzo[a,d]cyclohepten]-3-yl)-3-piperidinecarboxylate

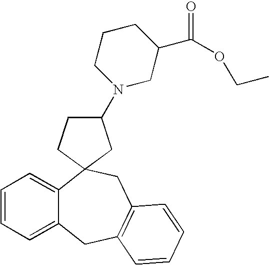

To a solution of (+)5′,11′-dihydro-3H-spiro[cyclopentane-1,10′-dibenzo[a,d]cyclohepten]-3-one (Intermediate 7, 70 mg, 0.267 mmol) and ethyl nipecotate (0.083 mL, 0.534 mmol) in 1,2-dichloroethane (4 mL) was added AcOH (0.076 mL, 1.334 mmol). The reaction mixture was stirred at room temperature for 1 hr. NaBH(OAC)₃ (85 mg, 0.400 mmol) was added and the resulting mixture was stirred at room temperature overnight. The mixture was washed with a saturated aqueous solution of NaHCO₃, then brine and the organic phase was separated and concentrated under vacuum. The crude mixture was purified on Si-Redisep (12 g) cartridge eluting with c-Hex:EtOAc (from 100:00 to 80:20) to afford 102 mg of the title compound as a mixture of two diastereoisomeric racemates; HPLC/MS Rf=3.16; m/z (ES): 404.1 [M+H]⁺

This isomeric mixture was submitted for chiral HPLC separation (Preparative chromatographic conditions: Column: Chiralcel OD-H; Mobile phase: n-Hexane/Isopropanol 90/10% v/v; Flow rate: 14 mL/min; UV: 220 nm) to give 2 single isomers (Compounds 29 and 30) plus a mixture of the other two (Compounds 31 and 32):

### Compound 29: Ethyl 1-(5′,11′-dihydrospiro[cyclopentane-1,10′-dibenzo[a,d]cyclohepten]-3-yl)-3-piperidinecarboxylate (isomer 3)

retention time=6.46 mins (5.5 mg); ¹H NMR (400 MHz, CHLOROFORM-d) d ppm 6.91-7.06 (m, 1H) 6.48-6.84 (m, 7H) 3.50-3.82 (m, 3H) 2.39-2.78 (m, 4H) 2.05-2.20 (m, 1H) 0.92-1.85 (m, 14H) 0.77 (t, 3H).

### Compound 30: Ethyl 1-(5′,11′-dihydrospiro[cyclopentane-1,10′-dibenzo[a,d]cyclohepten]-3-yl)-3-piperidinecarboxylate (isomer 4)

retention time=8.61 mins (3.9 mg); ¹H NMR (400 MHz, CHLOROFORM-d) d ppm 6.96-7.12 (m, 1H) 6.53-6.83 (m, 7H) 3.68-3.88 (m, 3H) 3.54-3.67 (m, 1H) 2.72-2.87 (m, 2H) 2.52-2.67 (m, 2H) 2.39-2.49 (m, 1H) 2.12-2.29 (m, 1H) 0.99-1.94 (m, 12H) 0.86 (t, 3H).

The mixture of the two other isomers (Compounds 31 and 32) was submitted to further chiral HPLC purification (Preparative chromatographic conditions: Column=Chiralcel OJ-H; Mobile phase=n-Hexane/Isopropanol 85/15% v/v; Flow rate=1 mL/min; DAD=210-340 nm) to obtain:

### Compound 31: Ethyl 1-(5′,11′-dihydrospiro[cyclopentane-1,10′-dibenzo[a,d]cyclohepten]-3-yl)-3-piperidinecarboxylate (isomer 1)

retention time=5.60 mins (35 mg); ¹H NMR (400 MHz, CHLOROFORM-d) d ppm 7.27-7.33 (m, 1H) 7.08-7.26 (m, 6H) 7.01-7.08 (m, 3H) 4.28 (d, 1H) 4.09-4.18 (m, 2H) 4.00 (d, 2H) 3.29 (d, 2H) 2.88-3.17 (m, 4H) 2.52-2.70 (m, 1H) 1.76-2.30 (m, 9H) 1.60-1.75 (m, 1H) 1.41-1.57 (m, 1H) 1.20 (t, 3H).

### Compound 32: Ethyl 1-(5′,11′-dihydrospiro[cyclopentane-1,10′-dibenzo[a,d]cyclohepten]-3-yl)-3-piperidinecarboxylate (isomer 2)

retention time=8.77 mins (31 mg); ¹H NMR (400 MHz, CHLOROFORM-d) d ppm 7.27-7.33 (m, 1H) 7.09-7.26 (m, 6H) 7.01-7.08 (m, 1H) 4.28 (d, 1H) 4.13-4.23 (m, 2H) 3.93-4.08 (m, 1H) 3.19-3.36 (m, 2H) 3.11 (d, 1H) 2.93-3.06 (m, 1H) 2.76-2.91 (m, 1H) 2.58-2.72 (m, 1H) 1.81-2.29 (m, 9H) 1.71-1.80 (m, 1H) 1.57-1.70 (m, 1H) 1.42-1.56 (m, 1H) 1.31 (t, 3H).

### Compound 33: Ethyl 1-(2′-fluoro-11′-oxo-11′H-spiro[cyclopentane-1,10′-dibenzo[b,f]thiepin]-3-yl)-4-piperidinecarboxylate

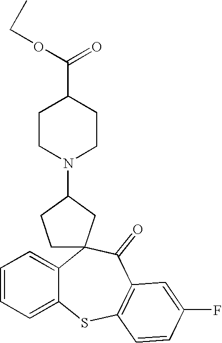

To a solution of 2′-fluoro-3H,11′H-spiro[cyclopentane-1,10′-dibenzo[b,f]thiepin]-3,11′-dione (Intermediate 15, 150 mg, 0.480 mmol) and ethyl 4-piperidinecarboxylate (91 mg, 0.576 mmol) in 1,2-dichloroethane (DCE) (5 mL) were added AcOH (0.055 mL, 0.960 mmol) and then NaBH(OAC)₃ (122 mg, 0.576 mmol) and the mixture was left to stir for 72 h. The mixture was quenched with NaHCO₃ solution and left to stir for 30 min. The mixture was diluted with DCM, the water phase separated and extracted with DCM. The combined organic layers were washed with brine and the solvent dried over Na₂SO₄. Solvent evaporation gave a crude product which was purified with Si25 cartridge (Horizon) eluting with cHex/EtOAC=7/3 to obtain the title compound as yellow oil (103 mg); UPLC/MS Rf=0.69; m/z (ES): 454.1 [M+H]⁺; ¹H NMR (400 MHz, CHLOROFORM-d) d ppm 7.70 (dd, 1H) 7.59-7.64 (m, 1H) 7.36-7.51 (m, 3H) 7.11-7.24 (m, 2H) 4.03-4.23 (m, 2H) 3.01-3.18 (m, 1H) 2.77-2.94 (m, 3H) 2.60-2.72 (m, 2H) 2.16-2.32 (m, 2H) 1.77-1.99 (m, 4H) 1.43-1.73 (m, 4H) 1.16-1.34 (m, 3H).

### Compound 34: Ethyl 1-(5′,11′-dihydrospiro[cyclopentane-1,10′-dibenzo[a,d]cyclohepten]-3-yl-4-fluoro-4-piperidinecarboxylate (diastereomeric mixture 2)

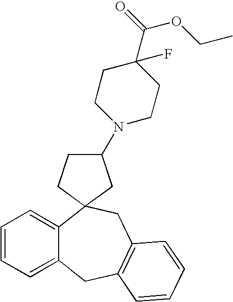

To a solution of (+)5′,11′-dihydro-3H-spiro[cyclopentane-1,10′-dibenzo[a,d]cyclohepten]-3-one (Intermediate 7, 58 mg, 0.221 mmol) and ethyl 4-fluoro-4-piperidinecarboxylate (prepared as described in WO 02/32893) (58.1 mg, 0.332 mmol) in 1,2-dichloroethane (3 mL) was added a drop of AcOH. The reaction mixture was stirred at room temperature for 0.5 h and then NaBH(OAC)₃ (70.3 mg, 0.332 mmol) was added. The resulting reaction mixture was stirred for 3 h, quenched with NaHCO₃ (saturated aqueous solution) and extracted with dichloromethane. The organic layers were combined, dried (Na₂SO₄) and concentrated in-vacuo. The crude product was purified by flash chromatography on silica gel (20 g) eluting with a gradient of dichloromethane/methanol 99.5/0.5 to 99/1 affording the title compound (30 mg) as a mixture of diastereoisomers (˜10/90).

This diastereomeric mixture was submitted for chiral HPLC purification (Preparative chromatographic conditions: Column=Chiralcel OJ-H; Mobile phase=n-Hexane/EtOH 85/15% v/v; Flow rate=0.8 mL/min; DAD=210-340 nm; CD=225 nm) to give two diastereoisomers:

### Compound 35: Ethyl 1-(5′,11′-dihydrospiro[cyclopentane-1,10′-dibenzo[a,d]cyclohepten]-3-yl-4-fluoro-4-piperidinecarboxylate (isomer 4)

retention time=19.9 minutes (6 mg); ¹H NMR (400 MHz, CHLOROFORM-d) d ppm 7.46 (d, 1H) 7.12-7.25 (m, 6H) 7.03-7.07 (m, 1H) 4.24-4.29 (m, 3H) 4.03 (d, 1H) 3.23 (d, 1H) 3.00-3.06 (m, 3H) 2.83-2.87 (m 1H) 1.82-2.43 (m, 12H) 1.32 (t, 3H). UPLC/MS Rf=0.71; m/z (ES): 422.2 [M+H]⁺.

### Compound 36: Ethyl 1-(5′,11′-dihydrospiro[cyclopentane-1,10′-dibenzo[a,d]cyclohepten]-3-yl)-4-fluoro-4-piperidinecarboxylate (isomer 2)

retention time=21.9 minutes (21 mg);

1H NMR (400 MHz, CHLOROFORM-d) d ppm 7.31-7.32 (m, 1H) 7.14-7.21 (m, 6H) 7.00-7.08 (m, 1H) 4.20-4.35 (m, 3H) 4.02 (d, 1H) 3.31 (d, 1H) 3.12 (d, 1H) 2.97-3.08 (m, 2H) 2.74-2.90 (m, 1H) 1.78-2.49 (m, 12H) 1.34 (t, 3H). UPLC/MS Rf=0.70; m/z (ES): 422.2 [M+H]⁺.

### Compound 37: Methyl 1-(5′,11′-dihydrospiro[cyclopentane-1,10′-dibenzo[a,d]cyclohepten]-3-yl)-4-fluoro-4-piperidinecarboxylate (diastereomeric mixture 2)

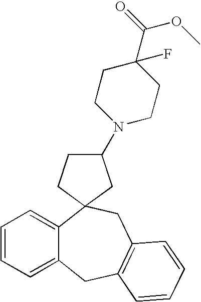

To a suspension of zinc chloride (14.81 mg, 0.109 mmol) in methanol (5 ml) under nitrogen, was added (+)5′,11′-dihydro-3H-spiro[cyclopentane-1,10′-dibenzo[a,d]cyclohepten]-3-one (Intermediate 7, 57 mg, 0.217 mmol) and ethyl 4-fluoro-4-piperidinecarboxylate (prepared as described in WO 02/32893) (57.1 mg, 0.326 mmol). After 1 h NaCNBH₄ (54.6 mg, 0.869 mmol) was added and the mixture was left to stir at room temperature for 24 h. During this time the mixture became clean and the conversion to methyl ester was observed. The reaction mixture was quenched with NaHCO₃ (saturated aqueous solution) and extracted with dichloromethane. The organic layers were combined, dried (Na₂SO₄) and concentrated in-vacuo. The crude product was purified by flash chromatography on silica gel (20 g) eluting with a gradient from dichloromethane/methanol 100%, 99.5/0.5 to 99/1 affording the title compound (78 mg) as a mixture of diastereoisomers (˜80/20).

This diastereomeric mixture was submitted for chiral HPLC purification (Preparative chromatographic conditions: Column=Chiralcel OD-H; Mobile phase=n-Hexane/Isopropanol 95/5% v/v; Flow rate=1 mL/min; DAD=210-340 nm; CD=225 nm) to give two diastereoisomers:

### Compound 38: Methyl 1-(5′,11′-dihydrospiro[cyclopentane-1,10′-dibenzo[a,d]cyclohepten]-3-yl)-4-fluoro-4-piperidinecarboxylate (isomer 2)

retention time=8.8 minutes (50 mg); 1H NMR (400 MHz, CHLOROFORM-d) d ppm 7.26-7.32 (m, 1H) 7.09-7.25 (m, 6H) 6.99-7.07 (m, 1H) 4.27 (d, 1H) 4.02 (d, 1H) 3.82 (s, 3H) 3.29 (d, 1H) 3.12 (d, 1H) 2.96-3.07 (m, 2H) 2.78-2.89 (m, 1H) 1.78-2.47 (m, 12H). UPLC/MS Rf=0.69; m/z (ES): 408.2 [M+H]⁺.

### Compound 39: Methyl 1-(5′,11′-dihydrospiro[cyclopentane-1,10′-dibenzo[a,d]cyclohepten]-3-yl)-4-fluoro-4-piperidinecarboxylate (isomer 4)

retention time=13.1 minutes (11 mg); 1H NMR (400 MHz, CHLOROFORM-d) d ppm 7.41-7.50 (m, 1H) 6.92-7.25 (m, 7H) 4.26 (d, 1H) 4.02 (d, 1H) 3.80 (s, 3H) 3.23 (d, 1H) 2.95-3.08 (m, 3H) 2.79-2.89 (m, 1H) 1.77-2.47 (m, 12H). UPLC/MS Rf=0.70; m/z (ES): 408.2 [M+H]⁺.

### Compound 40: Ethyl 4-(5′,11′-dihydrospiro[cyclopentane-1,10′-dibenzo[a,d]cyclohepten]-3-yl)-2-morpholinecarboxylate

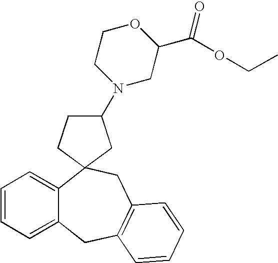

To a solution of (+)5′,11′-dihydro-3H-spiro[cyclopentane-1,10′-dibenzo[a,d]cyclohepten]-3-one (Intermediate 7, 70 mg, 0.267 mmol) and ethyl 2-morpholinecarboxylate (146 mg, 0.534 mmol) in 1,2-dichloroethane (DCE) (4 ml) under nitrogen was added AcOH (0.031 ml, 0.534 mmol). The reaction was stirred at room temperature for 1.5 h, NaBH(OAC)₃ (85 mg, 0.400 mmol) was added and the resulting mixture was stirred overnight. The mixture was diluted with DCM and the mixture washed with a saturated solution of NaHCO₃, then brine. The mixture was dried using a phase separator, and concentrated under vacuum. The crude mixture was purified on SiO₂ (regisep Cartridge 4g) eluting with cyclohexane:EtOAc (from 100:00 to 80:20) for 25 min and then with 20% of EtOAc for 30 min to give the title compound (89 mg) as a mixture of two diastereoisomeric racemates.

UPLC/MS Rt=0.67; m/z (ES): 406.1 [M+H]⁺

This isomeric mixture was submitted for chiral HPLC separation (Preparative chromatographic conditions: Column: Chiralpak AD-H, Mobile phase: n-Hexane/Ethanol 97/3% v/v, Flow rate: 1 mL/min; UV: 225 nm) to give 2 single isomers (Compound 41 and 42 plus a mixture of the other two (Compounds 43 and 44):

### Compound 41: Ethyl 4-(5′,11′-dihydrospiro[cyclopentane-1,10′-dibenzo[a,d]cyclohepten]-3-yl)-2-morpholinecarboxylate (isomer 1)

retention time=8.87 mins (28 mg); ¹H NMR (400 MHz, CHLOROFORM-d) d ppm 7.46-7.44 (m, 1H) 7.26-7.02 (m, 7H) 4.32-4.20 (m, 4H) 4.09-4.01 (m, 2H) 3.77-3.68 (m, 1H) 3.23-3.14 (m, 2H) 3.09-3.01 (m, 1H) 3.0-2.99 (m, 1H) 2.71-2.64 (m, 1H) 2.39-1.78 (m, 8H) 1.37-1.30 (t, 3H).

### Compound 42: Ethyl 4-(5′,11′-dihydrospiro[cyclopentane-1,10′-dibenzo[a,d]cyclohepten]-3-yl)-2-morpholinecarboxylate (isomer 4)

retention time=17.99 mins (8 mg); ¹H NMR (400 MHz, CHLOROFORM-d) d ppm 7.30-7.00 (m, 8H) 4.31-4.18 (m, 4H) 4.13-3.97 (m, 2H) 3.83-3.73 (m, 1H) 3.33-3.08 (m, 2H) 3.07-2.92 (m, 2H) 2.81-2.78 (m, 1H) 2.39-1.80 (m, 8H) 1.35-1.28 (t, 3H).

The mixture of the two isomers retention time=12.28 mins (35 mg), (Compounds 43 and 44 was submitted to further chiral HPLC purification (Preparative chromatographic conditions: Column: Chiralpak AD-H, Mobile phase: n-Hexane/Ethanol 97/3% v/v, Flow rate: 1 mL/min; UV: 225 nm) to obtain:

### Compound 43: Ethyl 4-(5′,11′-dihydrospiro[cyclopentane-1,10′-dibenzo[a,d]cyclohepten]-3-yl)-2-morpholinecarboxylate (isomer 3)

retention time=9.49 mins (4 mg); ¹H NMR (400 MHz, CHLOROFORM-d) d ppm 7.57-7.34 (m, 1H) 7.34-6.69 (m, 7H) 4.15-4.37 (m, 4H) 4.14-4.01 (m, 2H) 3.86-3.62 (m, 1H) 3.21-3.13 (m, 1H) 3.14-3.09 (m, 3H) 2.85-2.78 (m, 1H) 2.40-1.83 (m, 8H) 1.32-1.25 (t, 3H).

### Compound 44: Ethyl 4-(5′,11′-dihydrospiro[cyclopentane-1,10′-dibenzo[a,d]cyclohepten]-3-yl)-2-morpholinecarboxylate (isomer 2)

retention time=10.81 mins (28 mg); ¹H NMR (400 MHz, CHLOROFORM-d) d ppm 7.52-6.92 (m, 8H) 4.37-4.18 (m, 4H) 4.15-3.97 (m, 2H) 3.85-3.73 (m, 1H) 3.33-3.08 (m, 3H) 3.08-2.93 (m, 1H) 2.75-2.62 (m, 1H) 2.40-1.87 (m, 8H) 1.36-1.32 (t, 3H).

### Compound 45: Ethyl 3-(5′,11′-dihydrospiro[cyclopentane-1,10′-dibenzo[a,d]cyclohepten]-3-yl)-3-azabicyclo[3.1.0]hexane-1-carboxylate

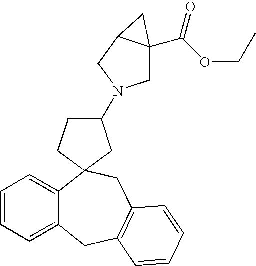

To a solution of (+)5′,11′-dihydro-3H-spiro[cyclopentane-1,10′-dibenzo[a,d]cyclohepten]-3-one (Intermediate 7, 70 mg, 0.267 mmol) and ethyl 3-azabicyclo[3.1.0]hexane-1-carboxylate (83 mg, 0.534 mmol, for preparation see WO 2007/055093) in 1,2-dichloroethane (5 mL) under nitrogen was added AcOH (0.076 mL, 1.334 mmol). The reaction mixture was stirred at room temperature for 1 h 30 min and then NaBH(OAC)₃ (85 mg, 0.400 mmol) was added. The resulting mixture was stirred overnight. The mixture was diluted with DCM, the organic phase was then washed with saturated solution of NaHCO₃, then brine and concentrated under vacuum. The crude product was purified through Si-redisep Cartridge (12 g) eluting with cHex:EtOAc (from 100:00 to 80:20) to afford 82 mg of the title compound as a mixture of two diastereoisomeric racemates;

UPLC/MS Rf=0.67; m/z (ES): 402.2 [M+H]⁺.

This mixture was submitted for chiral HPLC separation (Preparative chromatographic conditions: Column=Chiralcel OJ-H; Mobile phase=n-Hexane/Ethanol/Isopropanol 96/2/2% v/v′; Flow rate=1 mL/min; DAD=210-340 nm; CD=225 nm) to obtain:

### Compound 46: Ethyl 3-(5′,11′-dihydrospiro[cyclopentane-1,10′-dibenzo[a,d]cyclohepten]-3-yl)-3-azabicyclo[3.1.0]hexane-1-carboxylate (isomer 3)

retention time=9.77 mins (4.5 mg); ¹H NMR (400 MHz, CHLOROFORM-d) d ppm 7.41-7.53 (m, 1H) 7.09-7.23 (m, 6H) 6.97-7.07 (m, 1H) 4.05-4.23 (m, 4H) 2.91-3.20 (m, 5H) 2.66-2.73 (m, 1H) 2.44-2.54 (m, 1H) 1.77-2.24 (m, 7H) 1.40-1.51 (m, 1H) 1.30-1.37 (m, 1H) 1.20-1.29 (m, 3H).

### Compound 47: Ethyl 3-(5′,11′-dihydrospiro[cyclopentane-1,10′-dibenzo[a,d]cyclohepten]-3-yl)-3-azabicyclo[3.1.0]hexane-1-carboxylate (isomer 4)

retention time=11.35 mins (2.4 mg): ¹H NMR (400 MHz, CHLOROFORM-d) d ppm 7.42-7.54 (m, 1H) 7.09-7.25 (m, 6H) 6.97-7.06 (m, 1H) 4.03-4.25 (m, 4H) 2.91-3.22 (m, 5H) 2.65-2.75 (m, 1H) 2.40-2.52 (m, 1H) 1.77-2.24 (m, 7H) 1.41-1.51 (m, 1H) 1.31-1.38 (m, 1H) 1.22-1.31 (m, 3H).

### Compound 48: Ethyl 3-(5′,11′-dihydrospiro[cyclopentane-1,10′-dibenzo[a,d]cyclohepten]-3-yl)-3-azabicyclo[3.1.0]hexane-1-carboxylate (isomer 1)

retention time=13.62 mins (28 mg); ¹H NMR (400 MHz, CHLOROFORM-d) d ppm 7.29-7.34 (m, 1H) 7.08-7.24 (m, 6H) 7.00-7.07 (m, 1H) 4.22 (d, 1H) 4.14 (q, 2H) 4.04 (d, 1H) 3.21-3.29 (m, 1H) 3.08-3.16 (m, 3H) 2.96-3.06 (m, 1H) 2.68 (d, 1H) 2.41-2.51 (m, 1H) 1.77-2.15 (m, 7H) 1.44-1.51 (m, 1H) 1.29-1.37 (m, 1H) 1.25 (t, 3H).

### Compound 49: Ethyl 3-(5′,11′-dihydrospiro[cyclopentane-1,10′-dibenzo[a,d]cyclohepten]-3-yl)-3-azabicyclo[3.1.0]hexane-1-carboxylate (isomer 2)

retention time=17.76 mins (26 mg); ¹H NMR (400 MHz, CHLOROFORM-d) d ppm 7.26-7.32 (m, 1H) 7.08-7.25 (m, 6H) 6.98-7.07 (m, 1H) 4.12-4.26 (m, 3H) 4.05 (d, 1H) 3.21-3.29 (m, 2H) 3.14 (d, 1H) 2.97-3.07 (m, 2H) 2.78 (d, 1H) 2.33-2.42 (m, 1H) 1.80-2.13 (m, 7H) 1.43-1.54 (m, 1H) 1.33-1.39 (m, 1H) 1.28 (t, 3H).

### Compound 50: Methyl 1-(11′H-spiro[cyclopentane-1,10′-dibenzo[b,f]oxepin]-3-yl)-1,2,5,6-tetrahydro-3-pyridinecarboxylate

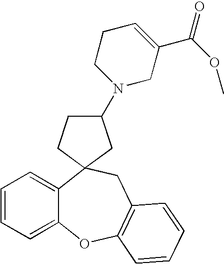

To 3H,11′H-spiro[cyclopentane-1,10′-dibenzo[b,f]oxepin]-3-one (Intermediate 20, 100 mg, 0.378 mmol) in DCE (5 ml) was added methyl 1,2,5,6-tetrahydro-3-pyridinecarboxylate (81 mg, 0.454 mmol) HCl salt and DIPEA (0.066 ml, 0.378 mmol). After stirring for 10 minutes sodium triacetoxyborohydride (120 mg, 0.567 mmol) and acetic acid (0.108 ml, 1.892 mmol) were added and the reaction left stirring overnight. The reaction was quenched with NaHCO₃, the organic layer separated and the water phase extracted with DCM. The combined organic layers were dried over Na₂SO₄ and the solvent evaporated to give the crude product (150 mg) as a mixture of two diastereoisomeric racemates.

This mixture was submitted for chiral HPLC separation (Preparative chromatographic conditions: Column=Chiralcel OJ-H; Mobile phase=n-Hexane/(Ethanol+0.1% isopropylamine) 93/7% v/v; Flow rate=14 mL/min; DAD=225 nm) to obtain:

### Compound 51: Methyl 1-(11′H-spiro[cyclopentane-1,10′-dibenzo[b,f]oxepin]-3-yl)-1,2,5,6-tetrahydro-3-pyridinecarboxylate (isomer 1)

retention time=11.70 mins (33 mg)

m/z (ES): 279.1 [M+H]⁺

¹H NMR (400 MHz, CHLOROFORM-d) δ ppm 7.27-7.34 (m, 1H) 7.01-7.23 (m, 8H) 3.76 (s, 3H) 3.18-3.39 (m, 3H) 3.07-3.16 (m, 1H) 2.91-3.02 (m, 1H) 2.60 (d, J=4.17 Hz, 2H) 2.44-2.53 (m, 1H) 2.35-2.42 (m, 2H) 2.01-2.24 (m, 3H) 1.93 (s, 2H).

### Compound 52: Methyl 1-(11′H-spiro[cyclopentane-1,10′-dibenzo[b,f]oxepin]-3-yl)-1,2,5,6-tetrahydro-3-pyridinecarboxylate (isomer 2)

retention time=20.27 mins (32 mg)

m/z (ES): 279.1 [M+H]⁺

¹H NMR (400 MHz, CHLOROFORM-d) δ ppm 7.27-7.34 (m, 1H) 7.01-7.23 (m, 8H) 3.76 (s, 3H) 3.18-3.39 (m, 3H) 3.07-3.16 (m, 1H) 2.91-3.02 (m, 1H) 2.60 (d, J=4.17 Hz, 2H) 2.44-2.53 (m, 1H) 2.35-2.42 (m, 2H) 2.01-2.24 (m, 3H) 1.93 (s, 2H).

### Compound 53: 1-(11′H-spiro[cyclopentane-1,10′-dibenzo[b,f]oxepin]-3-yl)-1,2,5,6-tetrahydro-3-pyridinecarboxylic acid (isomer 1)

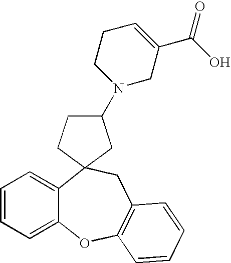

To a solution of methyl 1-(11′H-spiro[cyclopentane-1,10′-dibenzo[b,f]oxepin]-3-yl)-1,2,5,6-tetrahydro-3-pyridinecarboxylate (isomer 1, Compound 51, 33 mg, 0.085 mmol) in methanol (2 ml)/water (1 ml) lithium hydroxide (10.15 mg, 0.424 mmol) was added and the reaction was heated at 45° C. for 4 hours. The MeOH was evaporated, the water phase acidified with HCl (2N in water) until pH<1, a white solid precipitated. The suspension was purified by C18 cartridge (5 g) by using water and then MeOH as eluant to obtain, after solvent evaporation, the title compound was obtained as an off-white solid (16.7 mg).

m/z (ES): 376.2 [M+H]⁺

¹H NMR (400 MHz, CHLOROFORM-d) d ppm 7.26-7.34 (m, 1H) 6.91-7.22 (m, 8H) 3.51-2.36 (m, 15H).

### Compound 54: 1-(11′H-spiro[cyclopentane-1,10′-dibenzo[b,f]oxepin]-3-yl)-1,2,5,6-tetrahydro-3-pyridinecarboxylic acid (isomer 2)

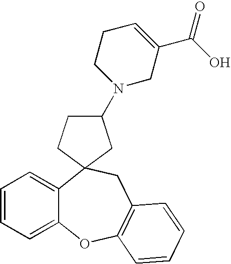

To a solution of methyl 1-(11′H-spiro[cyclopentane-1,10′-dibenzo[b,f]oxepin]-3-yl)-1,2,5,6-tetrahydro-3-pyridinecarboxylate (isomer 2, Compound 52, 32 mg, 0.082 mmol) in methanol (2 ml)/water (1 ml) was added lithium hydroxide (9.84 mg, 0.411 mmol) and the reaction was heated at 45° C. for 4 hours. The MeOH was evaporated, the water phase acidified with HCl (2N in water) until pH<1. The resulting suspension was purified by C18 cartridge (5 g) by using water and then MeOH as eluant to obtain, after solvent evaporation, the title compound as an off-white solid (11 mg).

m/z (ES): 376.2 [M+H]⁺

¹H NMR (400 MHz, CHLOROFORM-d) δ ppm 7.24-7.28 (m, 1H) 7.01-7.22 (m, 7H) 6.91-6.96 (m, 1H) 3.51-2.36 (m, 15H).

### Compound 55: Methyl 1-(11′H-spiro[cyclopentane-1,10′-dibenzo[b,f]oxepin]-3-yl)-1,2,3,6-tetrahydro-4-pyridinecarboxylate

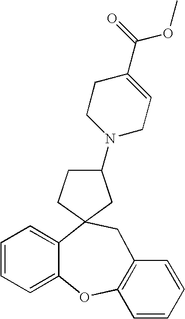

To 3H,11′H-spiro[cyclopentane-1,10′-dibenzo[b,f]oxepin]-3-one (Intermediate 20, 100 mg, 0.378 mmol) in DCE (5 ml) was added methyl 1,2,3,6-tetrahydro-4-pyridinecarboxylate (81 mg, 0.574 mmol) HCl salt and DIPEA (0.066 ml, 0.378 mmol) were added. After stirring for 10 minutes sodium triacetoxyborohydride (120 mg, 0.567 mmol) and acetic acid (0.108 ml, 1.892 mmol) were added and the reaction left stirring overnight. The reaction was quenched with NaHCO₃, the organic layer separated and the water phase extracted with DCM. The combined organic layers were dried over Na₂SO₄ and the solvent evaporated to give the title compound (150 mg) as a mixture of two diastereoisomeric racemates.

m/z (ES): 390.2 [M+H]⁺

This mixture was submitted for chiral HPLC separation (Preparative chromatographic conditions: Column=Chiralcel OJ-H; Mobile phase=n-Hexane/(Ethanol+0.1% isopropylamine) 93/7% v/v; Flow rate=14 mL/min; DAD=225 nm) to obtain:

### Compound 56: Methyl 1-(11′H-spiro[cyclopentane-1,10′-dibenzo[b,f]oxepin]-3-yl)-1,2,3,6-tetrahydro-4-pyridinecarboxylate (isomer 1)

retention time=17.23 mins (28 mg)

¹H NMR (400 MHz, CHLOROFORM-d) δ ppm 7.26-7.32 (m, 1H) 7.00-7.25 (m, 7H) 6.87-6.94 (m, 1H) 3.76 (s, 3H) 3.05-3.32 (m, 4H) 2.85-2.99 (m, 1H) 2.59-2.71 (m, 2H) 2.38-2.52 (m, 3H) 2.14 (d, J=6.19 Hz, 5H).

### Compound 57: Methyl 1-(11′H-spiro[cyclopentane-1,10′-dibenzo[b,f]oxepin]-3-yl)-1,2,3,6-tetrahydro-4-pyridinecarboxylate (isomer 2)

retention time=26.42 mins (27 mg)

¹H NMR (400 MHz, CHLOROFORM-d) δ ppm 7.27-7.33 (m, 1H) 7.01-7.24 (m, 7H) 6.87-6.95 (m, 1H) 3.76 (s, 3H) 3.06-3.31 (m, 4H) 2.83-2.99 (m, 1H) 2.60-2.70 (m, 2H) 2.38-2.51 (m, 3H) 2.14 (s, 5H).

### Compound 58: 1-(11′H-spiro[cyclopentane-1,10′-dibenzo[b,f]oxepin]-3-yl)-1,2,3,6-tetrahydro-4-pyridinecarboxylic acid (isomer 1)

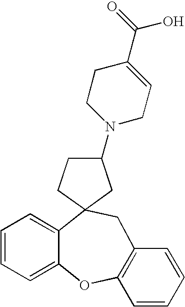

To a solution of methyl 1-(11′H-spiro[cyclopentane-1,10′-dibenzo[b,f]oxepin]-3-yl)-1,2,3,6-tetrahydro-4-pyridinecarboxylate (isomer 1, Compound 56, 28 mg, 0.072 mmol) in methanol (2 ml) was added water (1 ml) and lithium hydroxide (1.722 mg, 0.072 mmol) and the mixture heated at 45° C. for 4 hours. The MeOH was evaporated, the water phase acidified with HCl (2N in water) until pH<1. The suspension was purified by C18 cartridge (5 g) using water and then MeOH as eluant to obtain, after solvent evaporation, a cream solid (18 mg). The product was further purified by fraction Lynx HPLC to obtain the title compound as a white solid (7.5 mg); m/z (ES): 376.1 [M+H]⁺

¹H NMR (400 MHz, DMSO-d₆) δ ppm 7.32-7.39 (m, 1H) 7.04-7.30 (m, 7H) 6.74-6.84 (m, 1H) 3.40-2.20 (m, 15H).

### Compound 59: 1-(11′H-spiro[cyclopentane-1,10′-dibenzo[b,f]oxepin]-3-yl)-1,2,3,6-tetrahydro-4-pyridinecarboxylic acid (isomer 2)

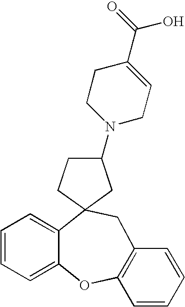

To a solution of methyl 1-(11′H-spiro[cyclopentane-1,10′-dibenzo[b,t]oxepin]-3-yl)-1,2,3,6-tetrahydro-4-pyridinecarboxylate (isomer 2, Compound 57, 27 mg, 0.069 mmol) in methanol (2 ml), water (1 ml) and lithium hydroxide (1.660 mg, 0.069 mmol) were added and the mixture heated at 45° C. for 4 hours. The MeOH was evaporated, the water phase acidified with HCl (2N in water) until pH<1. The suspension was purified by C18 cartridge (5 g) by using water and then MeOH as eluant to obtain, after solvent evaporation, a cream solid (17 mg). The product was further purified by fraction Lynx HPLC to give the title compound as a white solid (8.2 mg).

m/z (ES): 376.1 [M+H]⁺

¹H NMR (400 MHz, DMSO-d₆) δ ppm 7.32-7.39 (m, 1H) 7.04-7.30 (m, 7H) 6.74-6.84 (m, 1H) 3.40-2.20 (m, 15H).

### Compound 60: Methyl 4-fluoro-1-(11′H-spiro[cyclopentane-1,10′-dibenzo[b,f]oxepin]-3-yl)-4-piperidinecarboxylate

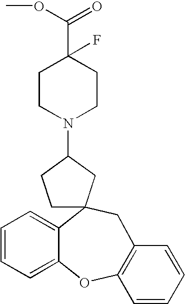

To a solution of 5′,11′-dihydro-3H-spiro[cyclopentane-1,10′-dibenzo[a,d]cyclohepten]-3-one (Intermediate 20, 114 mg, 0.435 mmol) in methanol (7 ml) was added ethyl 4-fluoro-4-piperidinecarboxylate (114 mg, 0.651 mmol, for preparation see International Patent publication WO 02/32893) and zinc chloride (30 mg, 0.220 mmol). The reaction was left stirring for 1 hr then sodium cyanoborohydride (110 mg, 1.750 mmol) was added and the mixture left at room temperature for 48 hrs. The reaction was quenched with NaHCO₃, diluted in DCM and the water phase separated and extracted back with DCM. The combined organic layers were washed with brine and then dried over Na₂SO₄. After solvent evaporation, the crude compound was obtained (110 mg). Purification was done by prep HPLC (Column=Gemini C18 AXIA, 50×21 mm, 5 μm; Mobile phase=A: NH₄HCO₃ sol. 10 mM, pH 10, B: CH₃CN; Gradient: 50% (B) for 1 min, 50% to 70% (B) in 9 min, 70% to 95% (B) in 0.5 min, 95% (B) for 2 min; Flow rate=17 mL/min; DAD=210-350 nM; Mass range: 100-900 amu), to obtain the title compound as a colourless wax (60 mg) as a mixture of two diastereoisomeric racemates.

m/z (ES): 410.0 [M+H]⁺

The stereoisomeric mixture (Compound 60) was submitted for chiral HPLC separation (Preparative chromatographic conditions: Column=Chiralcel OJ-H; Mobile phase=n-Hexane/Ethanol 70/30% v/v; Flow rate=14 mL/min; DAD=225 nm) to obtain:

### Compound 61: Methyl 4-fluoro-1-(11′H-spiro[cyclopentane-1,10′-dibenzo[b,f]oxepin]-3-yl)-4-piperidinecarboxylate (isomer 1)

retention time=10.8 mins, light yellow oil (19.5 mg)

m/z (ES): 410.0 [M+H]⁺

¹H NMR (400 MHz, CHLOROFORM-d) δ ppm 7.25-7.33 (m, 1H) 6.99-7.23 (m, 7H) 3.80 (s, 3H) 3.04-3.30 (m, 2H) 2.78-3.01 (m, 3H) 1.63-2.48 (m, 12H).

### Compound 62: Methyl 4-fluoro-1-(11′H-spiro[cyclopentane-1,10′-dibenzo[b,f]oxepin]-3-yl)-4-piperidinecarboxylate (isomer 2)

retention time=15.1 mins, light yellow oil (18.5 mg)

m/z (ES): 410.0 [M+H]⁺

¹H NMR (400 MHz, CHLOROFORM-d) δ ppm 7.25-7.32 (m, 1H) 6.99-7.23 (m, 7H) 3.80 (s, 3H) 3.04-3.29 (m, 2H) 2.78-3.03 (m, 3H) 1.63-2.49 (m, 12H).

### Compound 63: 4-Fluoro-1-(11′H-spiro[cyclopentane-1,10′-dibenzo[b,f]oxepin]-3-yl)-4-piperidinecarboxylic acid (isomer 1)

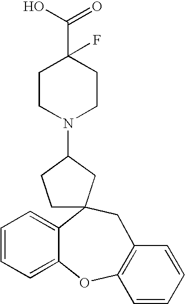

To a solution of methyl 4-fluoro-1-(11′H-spiro[cyclopentane-1,10′-dibenzo[b,f]oxepin]-3-yl)-4-piperidinecarboxylate (isomer 1, Compound 61, 19.5 mg, 0.048 mmol) in methanol (2 ml) were added water (2 ml) and lithium hydroxide (5.70 mg, 0.238 mmol) and the mixture heated at 50° C. for 4 hours. The MeOH was evaporated and the crude was purified by C18 cartridge (5 g) by using water and then MeOH as eluant to obtain, after solvent evaporation, the title compound as a white solid (14.5 mg, Lithium salt).

RT=0.60; m/z (ES): 396.1 [M+H]⁺

¹H NMR (400 MHz, DMSO-d₆) δ ppm 7.30-7.37 (m, 1H) 7.24-7.30 (m, 1H) 7.01-7.23 (m, 6H) 1.48-3.41 (m, 15H).

### Compound 64: 4-Fluoro-1-(11′H-spiro[cyclopentane-1,10′-dibenzo[b,f]oxepin]-3-yl)-4-piperidinecarboxylic acid (isomer 2)

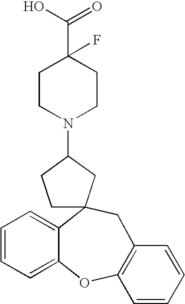

To a solution of methyl 4-fluoro-1-(11′H-spiro[cyclopentane-1,10′-dibenzo[b,t]oxepin]-3-yl)-4-piperidinecarboxylate (isomer 2, Compound 62, 18.5 mg, 0.045 mmol) in methanol (2 ml) were added water (2 ml) and lithium hydroxide (5.41 mg, 0.226 mmol) and the mixture heated at 50° C. for 4 hours. The MeOH was evaporated and the crude was purified by C18 cartridge (5 g) by using water and then MeOH as eluant to obtain, after solvent evaporation, the title compound as a white solid (15 mg, lithium salt).

RT=0.61; m/z (ES): 396.1 [M+H]⁺

¹H NMR (400 MHz, DMSO-d₆) δ ppm 7.30-7.38 (m, 1H) 7.24-7.30 (m, 1H) 7.04-7.23 (m, 6H) 1.48-3.44 (m, 15H).

### Compound 65: Methyl 1-(5′,11′-dihydrospiro[cyclopentane-1,10′-dibenzo[a,d]cyclohepten]-3-yl)-1,2,3,6-tetrahydro-4-pyridinecarboxylate

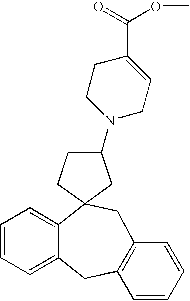

To a DCE (5 ml) solution of 5′,11′-Dihydro-3H-spiro[cyclopentane-1,10′-dibenzo[a,d]cyclohepten]-3-one (Intermediate 5, 100 mg, 0.378 mmol) were added methyl 1,2,3,6-tetrahydro-4-pyridinecarboxylate (0.969 g, 6.86 mmol) and Acetic acid (1.717 g, 28.6 mmol). After stirring for 10 minutes sodium triacetoxyborohydride (120 mg, 0.567 mmol) was added and the reaction left at room temperature for 56 hrs. The reaction was quenched with NaHCO₃ saturated solution, the organic layer separated and the water phase extracted with DCM. The combined organic layers were dried over Na₂SO₄ and the solvent evaporated to give a yellow solid which was then purified by column chromatography (Biotage SP1, 40M cartridge, DCM to DCM/MeOH=9/1 as eluant) to give the title compound as a mixture of two diastereoisomeric racemates (1.9 g).

This mixture was then submitted for chiral HPLC for further purification to obtain the two single major isomers (Preparative chromatographic conditions: Column=Chiralcel OD-H; Mobile phase=n-Hexane/2-Propanol 95/5% v/v; Flow rate=14 mL/min; DAD=225 nm).

### Compound 66: Methyl 1-(5′,11′-dihydrospiro[cyclopentane-1,10′-dibenzo[a,d]cyclohepten]-3-yl)-1,2,3,6-tetrahydro-4-pyridinecarboxylate (isomer 1)

retention time=12.8 mins (610 mg)

¹H NMR (400 MHz, DMSO-d₆) δ ppm 7.07-7.32 (m, 7H) 6.98-7.05 (m, 1H) 6.82-6.91 (m, 1H) 3.95-4.27 (m, 2H) 3.68 (s, 3H) 2.98-3.31 (m, 5H) 2.55-2.63 (m, 2H) 2.23-2.37 (m, 2H) 1.67-2.13 (m, 6H).

### Compound 67: Methyl 1-(5′,11′-dihydrospiro[cyclopentane-1,10′-dibenzo[a,d]cyclohepten]-3-yl)-1,2,3,6-tetrahydro-4-pyridinecarboxylate (isomer 2)

retention time=15.2 mins (530 mg)

¹H NMR (400 MHz, DMSO-d₆) δ ppm 7.08-7.32 (m, 7H) 6.98-7.06 (m, 1H) 6.81-6.90 (m, 1H) 3.93-4.27 (m, 2H) 3.68 (s, 3H) 3.00-3.31 (m, 5H) 2.54-2.63 (m, 2H) 2.24-2.37 (m, 2H) 1.67-2.10 (m, 6H).

### Compound 68: 1-(5′,11′-Dihydrospiro[cyclopentane-1,10′-dibenzo[a,d]cyclohepten]-3-yl)-1,2,3,6-tetrahydro-4-pyridinecarboxylic acid (isomer 2)

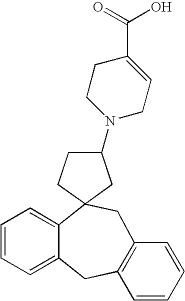

To a solution of methyl 1-(5′,11′-dihydrospiro[cyclopentane-1,10′-dibenzo[a,d]cyclohepten]-3-yl)-1,2,3,6-tetrahydro-4-pyridinecarboxylate (isomer 2, Compound 67, 280 mg, 0.723 mmol) in methanol (10 ml)/water (10.00 ml) lithium hydroxide (26.0 mg, 1.084 mmol) were added and the reaction was heated at 65° C. for 48 hours. The MeOH was evaporated and the water phase was purified by C18 cartridge (50 g) by using water and then MeOH as eluant to obtain, after solvent evaporation, the crude product (130 mg) as a light brown solid. This solid was further triturated in EtOH to get after filtration the title compound as a white solid (20 mg).

m/z (ES): 374.3 [M+H]⁺

¹H NMR (400 MHz, DMSO-d6) δ ppm 7.08-7.33 (m, 7H) 6.96-7.06 (m, 1H) 6.72-6.83 (m, 1H) 3.95-4.27 (m, 2H) 2.97-3.28 (m, 5H) 2.51 (m, 9H).

### Compound 69: 1-(5′,11′-Dihydrospiro[cyclopentane-1,10′-dibenzo[a,d]cyclohepten]-3-yl)-1,2,3,6-tetrahydro-4-pyridinecarboxylic acid (isomer 1)

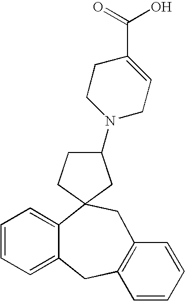

To a solution of methyl 1-(5′,11′-dihydrospiro[cyclopentane-1,10′-dibenzo[a,d]cyclohepten]-3-yl)-1,2,3,6-tetrahydro-4-pyridinecarboxylate (isomer 1, Compound 66, 610 mg, 1.574 mmol) in methanol (20 ml)/water (10 ml) was added lithium hydroxide (188 mg, 7.87 mmol) and the reaction was heated at 45° C. for 4 hours. The MeOH was evaporated, the water phase acidified with HCl (2N in water) until pH˜1, a white solid precipitated. This suspension was purified by C18 cartridge (25 g) by using water and then MeOH as eluant to obtain, after solvent evaporation, the title compound as a light cream powder (163 mg).

RT=0.63; m/z (ES): 374.09 [M+H]⁺

¹H NMR (400 MHz, DMSO-d₆) δ ppm 7.07-7.33 (m, 7H) 6.95-7.07 (m, 1H) 6.75-6.86 (m, 1H) 3.95-4.27 (m, 2H) 2.98-3.25 (m, 2H) 1.79-2.72 (m, 12H)

### Compound 70: Methyl 1-(5′,11′-dihydrospiro[cyclopentane-1,10′-dibenzo[a,d]cyclohepten]-3-yl)-3-azetidinecarboxylate

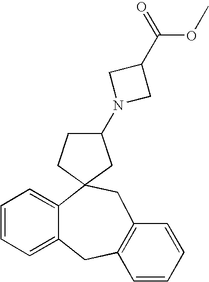

In a 100 mL round-bottomed flask was 5′,11′-dihydro-3H-spiro[cyclopentane-1,10′-dibenzo[a,d]cyclohepten]-3-one (Intermediate 5, 120 mg, 0.457 mmol), methyl 3-azetidinecarboxylate hydrochloride (104 mg, 0.686 mmol), DIPEA (0.088 ml, 0.503 mmol), and AcOH (0.131 ml, 2.287 mmol) in DCM (5 ml) to give a colorless solution. After stirring for 1 hr at room temperature NaBH(OAC)₃ (145 mg, 0.686 mmol) was added and the reaction mixture stirred overnight. NaHCO₃ was added, the phases separated and the organic washed with water. The aqueous was extracted with DCM. The phases were separated on a phase separator cartridge. The combined organic extracts evaporated to give the title compound as a mixture of two diastereoisomeric racemates (206 mg).

UPLC/MS Rf=1.07; m/z (ES): 362.18 [M+H]⁺

The diastereomeric mixture was submitted for chiral HPLC purification (Preparative chromatographic conditions: Column=Chiralcel OD-H; Mobile phase=n-Hexane/Ethanol 95/5% v/v; Flow rate=0.8 mL/min; DAD=210-340 nm; CD=230 nm) to give 2 single isomers plus a mixture of the other two:

### Compound 71: Methyl 1-(5′,11′-dihydrospiro[cyclopentane-1,10′-dibenzo[a,d]cyclohepten]-3-yl)-3-azetidinecarboxylate (isomer 1)

retention time=8.36 mins (66 mg)

¹H NMR (400 MHz, CHLOROFORM-d) d ppm 7.35-6.92 (m, 8H); 4.25-4.0 (q, 2H); 3.76 (s, 3H); 3.7-3.05 (m, 8H); 2.15-1.65 (m, 6H)

### Compound 72: Methyl 1-(5′,11′-dihydrospiro[cyclopentane-1,10′-dibenzo[a,d]cyclohepten]-3-yl)-3-azetidinecarboxylate (isomer 4)

retention time=12.06 mins (15 mg)

m/z (ES): 362.0 [M+H]⁺

The mixture of the two isomers: retention time=9.35 mins (76 mg) was submitted to further chiral HPLC purification (Preparative chromatographic conditions: Column=Chiralcel OD-H; Mobile phase=n-Hexane/2-Propanol 95/5% v/v; Flow rate=1.0 mL/min; DAD=210-340 nm; CD=230 nm) to obtain:

### Compound 73: Methyl 1-(5′,11′-dihydrospiro[cyclopentane-1,10′-dibenzo[a,d]cyclohepten]-3-yl)-3-azetidinecarboxylate (isomer 2)

retention time=8.32 mins (53 mg)¹H NMR (400 MHz, CHLOROFORM-d) d ppm 7.36-6.92 (m, 8H); 4-27-4.05 (q, 2H) 3.76 (s, 3H); 3.68-3.06 (m, 8H); 2.19-1.64 (m, 6H)

### Compound 74: Methyl 1-(5′,11′-dihydrospiro[cyclopentane-1,10′-dibenzo[a,d]cyclohepten]-3-yl)-3-azetidinecarboxylate (isomer 3)

retention time=9.75 mins (14.6 mg)

m/z (ES): 362.0 [M+H]⁺

### Compound 75: 1-(5′,11′-Dihydrospiro[cyclopentane-1,10′-dibenzo[a,d]cyclohepten]-3-yl)-3-azetidinecarboxylic acid formate salt (isomer 1)

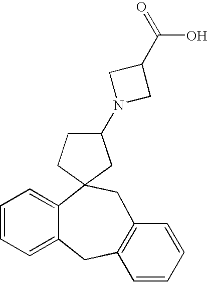

In a 50 mL round-bottomed flask was dissolved methyl 1-(5′,11′-dihydrospiro[cyclopentane-1,10′-dibenzo[a,d]cyclohepten]-3-yl)-3-azetidinecarboxylate (isomer 1, Compound 71, 66 mg, 0.183 mmol) in methanol (5 ml) and water (2.5 ml) to give a colorless solution. KOH (41.0 mg, 0.730 mmol) was added and the reaction was stirred at room temperature overnight. MS monitoring showed that the reaction was complete. Solvent was removed and the residue taken up with HCl 1M and passed through an HLB 6 g column (water and MeOH to elute) then a second purification was done by Fraction Lynx acid method to give the title compound as a yellow solid (57.9 mg).

UPLC/MS RT=0.58; m/z (ES): 348.08 [M+H]⁺

¹H NMR (400 MHz, DMSO-d) d ppm 7.33-6.94 (m, 8H); 4.17-3.99 (m, 2H); 3.48-3.37 (m, 2H); 3.26-3.15 (m, 5H); 3.14-3.06 (m, 1H); 2.01-1.90 (m, 2H); 1.85-1.61 (m, 4H)

### Compound 75A: 1-(5′,11′-dihydrospiro[cyclopentane-1,10′-dibenzo[a,d]cyclohepten]-3-yl)-3-azetidinecarboxylic acid (isomer 1)

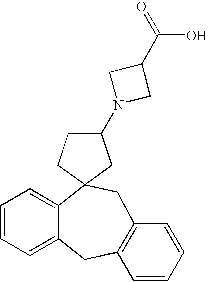

To a solution of methyl 1-(5′,11′-dihydrospiro[cyclopentane-1,10′-dibenzo[a,d]cyclohepten]-3-yl)-3-azetidinecarboxylate (isomer 1, Compound 71, 208 mg, 0.575 mmol) in methanol (8 ml) and water (4 ml), was added KOH (1M in MeOH) (2.302 ml, 2.302 mmol) and the reaction was stirred 2 hours. The solvent was concentrated at reduced pressure, the residue was dissolved in water and neutralized with HCl (1M, ˜2.5 ml), the product was purified by C18 column (25 g) to give the title compound (199 mg) as a white solid;

UPLC/MS RT=0.58; m/z (ES): 348.08 [M+H]⁺; ¹H NMR (400 MHz, CHLOROFORM-d) d ppm 7.41-7.51 (m, 1H) 6.98-7.25 (m, 7H) 4.11 (m, 7H) 3.60-3.85 (m, 1H) 3.31 (d, J=3.16 Hz, 2H) 2.14-2.44 (m, 5H) 1.90-2.06 (m, 1H)

### Compound 75B: (−)1-(5′,11′-Dihydrospiro[cyclopentane-1,10′-dibenzo[a,d]cyclohepten]-3-yl)-3-azetidinecarboxylic acid HCl salt

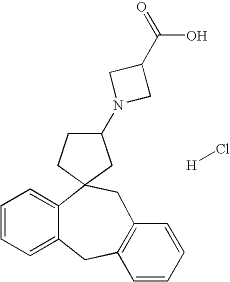

To a suspension of 1-(5′,11′-dihydrospiro[cyclopentane-1,10′-dibenzo[a,d]cyclohepten]-3-yl)-3-azetidinecarboxylic acid (isomer 1, Compound 75A, 150 mg, 0.432 mmol) in Tetrahydrofuran (THF) (4 mL), was added HCl (1M in Et2O) (0.863 mL, 0.863 mmol). After 12 hours the volatiles were removed to give the title compound (152 mg);

UPLC/MS RT=0.58; m/z (ES): 348.15 [M+H]⁺, [α]_{D}²⁰=−11.3° (c=0.53, MeOH),

1H NMR (400 MHz, DMSO-d6) d ppm 13.20 (br. s., 1H) 11.04 (br. s., 1H) 6.94-7.55 (m, 8H) 3.96-4.45 (m, 7H) 3.55-3.75 (m, 1H) 3.07-3.27 (m, 2H) 1.74-2.30 (m, 6H).

### Compound 76: 1-(5′,11′-Dihydrospiro[cyclopentane-1,10′-dibenzo[a,d]cyclohepten]-3-yl)-3-azetidinecarboxylic acid (isomer 2)

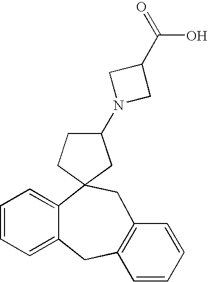

In a 50 mL round-bottomed flask was dissolved methyl 1-(5′,11′-dihydrospiro[cyclopentane-1,10′-dibenzo[a,d]cyclohepten]-3-yl)-3-azetidinecarboxylate (isomer 2, Compound 73, 53 mg, 0.147 mmol) in methanol (5 ml) and water (2.5 ml) to give a colorless solution. KOH (32.9 mg, 0.586 mmol) was added and the reaction was stirred at room temperature overnight. MS monitor showed that the reaction was complete. The solvents were removed and the residue taken up with HCl 1M and passed through an HLB 6 g column (water and MeOH to elute) to give the title compound (50 mg) as a white solid;

UPLC/MS RT=0.59; m/z (ES): 348.08 [M+H]⁺; ¹H NMR (400 MHz, CHLOROFORM-d) d ppm 7.58-7.34 (m, 1H); 7.23-6.91 (m, 7H); 4.80-3.64 (m, 8H); 3.36-3.14 (m, 2H); 2.41-1.75 (m, 6H).

### Compound 77: Methyl 1-(5′,11′-dihydrospiro[cyclopentane-1,10′-dibenzo[a,d]cyclohepten]-3-yl)-3-methyl-3-pyrrolidinecarboxylate

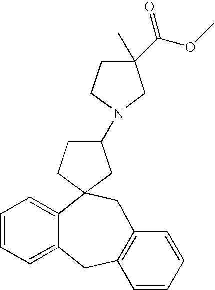

In a 100 mL round-bottomed flask was (+)5′,11′-dihydro-3H-spiro[cyclopentane-1,10′-dibenzo[a,d]cyclohepten]-3-one (Intermediate 7, 50 mg, 0.191 mmol), methyl 3-methyl-3-pyrrolidinecarboxylate hydrochloride (Intermediate 23, 51.4 mg, 0.286 mmol), DIPEA (0.037 ml, 0.210 mmol) and AcOH (0.055 ml, 0.953 mmol) in DCM (5 ml) to give a colorless solution. After stirring for 1 hr at room temperature NaBH(OAC)₃ (60.6 mg, 0.286 mmol) was added and the reaction mixture stirred overnight. MS monitor showed that the reaction was complete. NaHCO₃ was added, the phases separated and the organic washed with water. The aqueous phase was extracted with DCM. The phases were then separated on a phase separator cartridge. The combined organic extracts were evaporated to give the crude which was purified by Fraction Lynx acid method to give the title compound (90 mg) as a mixture of two diastereoisomeric racemates.

UPLC/MS RT=0.61; m/z (ES): 390.16 [M+H]⁺

The diastereomeric mixture was submitted for chiral HPLC purification (Preparative chromatographic conditions: Column=Whelk O1(R,R); Mobile phase=n-Hexane/2-Propanol 96/4% v/v; Flow rate=14.0 mL/min; CD=215 nm) to give 1 single isomer (Compound 78) plus a mixture of the other 3 isomers (Compound 79):

### Compound 78: Methyl 1-(5′,11′-dihydrospiro[cyclopentane-1,10′-dibenzo[a,d]cyclohepten]-3-yl-3-methyl-3-pyrrolidinecarboxylate (isomer 1)

retention time=16.3 mins (25 mg)

For major isomer of the mixture: ¹H NMR (400 MHz, CHLOROFORM-d) d ppm 7.34-7.29 (m, 1H); 7.26-7.01 (m, 7H); 4.30-3.74 (m, 2H); 3.74 (s, 3H); 3.32-3.14 (m, 2H); 3.11-2.83 (m, 2H); 2.77-2.41 (m, 4H); 2.19-1.63 (m, 7H); 1.41 (s, 3H)

### Compound 79: Methyl 1-(5′,11′-dihydrospiro[cyclopentane-1,10′-dibenzo[a,d]cyclohepten]-3-yl-3-methyl-3-pyrrolidinecarboxylate (diastereomeric mixture 4)

retention time=18.42 mins (18 mg)

¹H NMR (400 MHz, CHLOROFORM-d) d ppm 7.34-7.30 (m, 1H); 7.26-7.01 (m, 7H); 4.29-4.0 (m, 2H); 3.75 (s, 3H); 3.35-3.08 (m, 3H); 2.98-2.87 (m, 1H); 2.74-2.63 (m, 2H); 2.52-2.48 (m, 1H); 2.48-2.41 (m, 1H); 2.15-1.82 (m, 6H); 1.75-1.67 (m, 1H); 1.42 (s, 3H)

### Compound 80: 1-(5′,11′-Dihydrospiro[cyclopentane-1,10′-dibenzo[a,d]cyclohepten]-3-yl)-3-methyl-3-pyrrolidinecarboxylic acid (isomer 1)

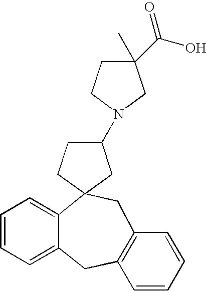

In a 50 mL round-bottomed flask was added methyl 1-(5′,11′-dihydrospiro[cyclopentane-1,10′-dibenzo[a,d]cyclohepten]-3-yl)-3-methyl-3-pyrrolidinecarboxylate (isomer 1, Compound 78, 25 mg, 0.064 mmol) and KOH (14.40 mg, 0.257 mmol) in methanol (1.5 ml) and water (0.375 ml) to give a colorless solution. The reaction mixture was heated in a microwave reactor (Personal Chemistry) for 30 min at 90° C. The solvents were removed and the residue taken up with HCl 1M and passed through an HLB 6 g column (water and MeOH to elute) to give the title compound as a white solid (21 mg);

UPLC/MS RT=0.63; m/z (ES): 376.13 [M+H]⁺; ¹H NMR (400 MHz, CHLOROFORM-d) d ppm 7.37-7.23 (m, 1H); 7.21-6.92 (m, 7H); 4.18-3.83 (m, 3H); 3.46-2.85 (m, 4H); 2.73-157 (m, 10H); 1.44 (s, 3H)

### Compound 81: 1-(5′,11′-Dihydrospiro[cyclopentane-1,10′-dibenzo[a,d]cyclohepten]-3-yl)-3-methyl-3-pyrrolidinecarboxylic acid (diastereomeric mixture 4)

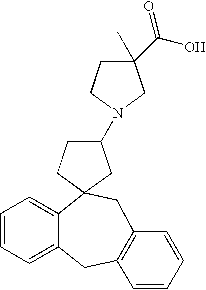

In a 50 mL round-bottomed flask was added methyl 1-(5′,11′-dihydrospiro[cyclopentane-1,10′-dibenzo[a,d]cyclohepten]-3-yl)-3-methyl-3-pyrrolidinecarboxylate (diastereomeric mixture 4, Compound 79, 18 mg, 0.046 mmol) and KOH (10.37 mg, 0.185 mmol) in methanol (1.5 ml) and water (0.375 ml) to give a colorless solution. The reaction mixture was heated in a microwave reactor (Personal Chemistry) for 30 min at 90° C. The solvents were removed and the residue taken up with HCl 1M and passed through an HLB 6 g column (water and MeOH to elute) to give the title compound (15 mg) as a white solid;

UPLC/MS RT=0.64; m/z (ES): 376.13 [M+H]⁺; ¹H NMR (400 MHz, CHLOROFORM-d) d ppm 7.44-7.32 (m, 1H); 7.25-6.97 (m, 7H); 4.21-4.03 (m, 3H); 3.76-3.55 (m, 2H); 3.37-3.20 (m, 3H); 2.84-2.46 (m, 4H); 2.32-2.08 (m, 3H); 1.97-1.75 (m, 2H); 1.44 (s, 3H)

### Compound 82: Methyl 1-(5′,11′-dihydrospiro[cyclopentane-1,10′-dibenzo[a,d]cyclohepten]-3-yl)-3-pyrrolidinecarboxylate

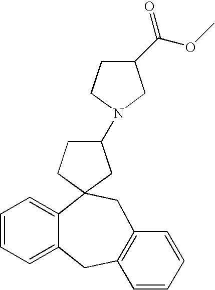

In a 100 mL round-bottomed flask was (+)5′,11′-dihydro-3H-spiro[cyclopentane-1,10′-dibenzo[a,d]cyclohepten]-3-one (Intermediate 7, 50 mg, 0.191 mmol), methyl 3-pyrrolidinecarboxylate (36.9 mg, 0.286 mmol), DIPEA (0.037 ml, 0.210 mmol) and AcOH (0.055 ml, 0.953 mmol) in DCM (5 ml) to give a colorless solution. After stirring for 1 hr at room temperature, NaBH(OAC)₃ (60.6 mg, 0.286 mmol) was added and the reaction mixture stirred overnight. NaHCO₃ was added, the phases separated and the organic washed with water. The aqueous was extracted with DCM. The phases were then separated on a phase separator cartridge. The combined organic extracts were evaporated. MS monitor after work up showed some acid formation so HCl in MeOH 1M 1 ml was added and the mixture stirred overnight at room temperature. MS monitor showed no more acid was present so solvent was removed NaHCO₃ was added and the aqueous extracted with DCM. The organic solvent was removed to give the crude which was purified by Fraction Lynx (Preparative chromatographic conditions: Column=Gemini C18 AXIA, 50×21 mm, 5 μm; Mobile phase=NH₄HCO₃sol. 10 mM, pH=10, Acetonitrile; Flow rate=17.0 mL/min; DAD=210-350 nm, ionization: ES⁺, mass range: 100-900 amu) to give the title compound (100 mg) as a mixture of two diastereoisomeric racemates.

UPLC/MS RT=0.63; m/z (ES): 376.13 [M+H]⁺

The diastereomeric mixture was submitted for chiral HPLC purification (Preparative chromatographic conditions: Column=Chiralcel OD-H; Mobile phase=n-Hexane/Ethanol 80/20% v/v; Flow rate=0.8 mL/min; DAD=225 nm, CD=225 nm) to give 2 single isomers plus a mixture of the other 2 isomers:

### Compound 83: Methyl 1-(5′,11′-dihydrospiro[cyclopentane-1,10′-dibenzo[a,d]cyclohepten]-3-yl)-3-pyrrolidinecarboxylate (isomer 3)

retention time=7.52 mins (7.3 mg)

m/z (ES): 376.1 [M+H]⁺

### Compound 84: Methyl 1-(5′,11′-dihydrospiro[cyclopentane-1,10′-dibenzo[a,d]cyclohepten]-3-yl)-3-Pyrrolidinecarboxylate (isomer 4)

retention time=8.25 mins (8.2 mg)

m/z (ES): 376.1 [M+H]⁺

The mixture of the two isomers (retention time=6.81 mins 43 mg) was submitted to further chiral HPLC purification (Preparative chromatographic conditions: Column=Chiralcel OD-H; Mobile phase=n-Hexane/2-Propanol 96/4% v/v; Flow rate=1.0 mL/min; DAD=225 nm) to obtain:

### Compound 85: Methyl 1-(5′,11′-dihydrospiro[cyclopentane-1,10′-dibenzo[a,d]cyclohepten]-3-yl)-3-pyrrolidinecarboxylate (isomer 1)

retention time=10.51 mins (18.7 mg)

¹H NMR (400 MHz, CHLOROFORM-d) d ppm 7.28-7.33 (m, 1H); 7.25-7.00 (m, 7H); 4-29-3.98 (q, 2H) 3.72 (s, 3H); 3.33-2.89 (m, 5H); 2.80-2.48 (m, 3H); 2.22-1.83 (m, 8H)

### Compound 86: Methyl 1-(5′,11′-dihydrospiro[cyclopentane-1,10′-dibenzo[a,d]cyclohepten]-3-yl)-3-pyrrolidinecarboxylate (isomer 2)

retention time=11.44 mins (16.6 mg)

¹H NMR (400 MHz, CHLOROFORM-d) d ppm 7.32-7.29 (m, 1H); 7.25-7.0 (m, 7H); 4.33-3.95 (q, 2H) 3.72 (s, 3H); 3.35-2.80 (m, 6H); 2.70-2.46 (m, 2H); 2.26-1.84 (m, 8H)

### Compound 87: 1-(5′,11′-Dihydrospiro[cyclopentane-1,10′-dibenzo[a,d]cyclohepten]-3-yl)-3-Pyrrolidinecarboxylic acid (isomer 1)

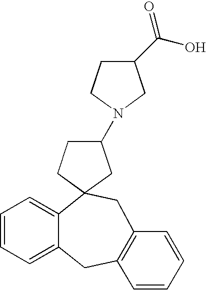

In a 50 mL round-bottomed flask was added methyl 1-(5′,11′-dihydrospiro[cyclopentane-1,10′-dibenzo[a,d]cyclohepten]-3-yl)-3-pyrrolidinecarboxylate (isomer 1, Compound 85, 18.7 mg, 0.050 mmol) and KOH (11.18 mg, 0.199 mmol) in methanol (3 ml) and water (0.75 ml) to give a colorless solution. The reaction was stirred at room temperature overnight. UPLC-MS monitoring showed that the reaction was complete. The solvents were removed and the residue taken up with HCl 1M and passed through an HLB 6 g column (water and MeOH to elute) to give the title compound (20 mg) as a white solid;

UPLC/MS RT=0.63; m/z (ES): 362.11 [M+H]⁺; ¹H NMR (400 MHz, CHLOROFORM-d) d ppm 7.48-7.4 (m, 1H); 7.22-7.0 (m, 7H); 4.19-4.0 (m, 2H); 3.82-3.18 (m, 8H); 2.69-1.84 (m, 8H)

### Compound 88: 1-(5′,11′-Dihydrospiro[cyclopentane-1,10′-dibenzo[a,d]cyclohepten]-3-yl)-3-pyrrolidinecarboxylic acid (isomer 2)

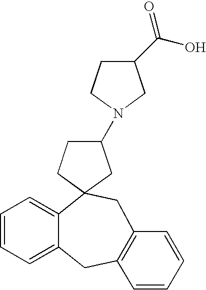

In a 50 mL round-bottomed flask was added methyl 1-(5′,11′-dihydrospiro[cyclopentane-1,10′-dibenzo[a,d]cyclohepten]-3-yl)-3-pyrrolidinecarboxylate (isomer 2, Compound 86, 16.6 mg, 0.044 mmol) and KOH (9.92 mg, 0.177 mmol) in methanol (3 ml) and water (0.75 ml) to give a colorless solution. The reaction was stirred at room temperature overnight. UPLC-MS monitoring showed that the reaction was complete. The solvents were removed and the residue taken up with HCl 1M and passed through an HLB 6 g column (water and MeOH to elute) to give the title compound (14 mg) as a white solid;

UPLC/MS RT=0.63; m/z (ES): 362.11 [M+H]⁺¹H NMR (400 MHz, CHLOROFORM-d) d ppm 1.86-1.49 (two bs, 1H); 7.61-7.45 (m, 1H); 7.23-6.90 (m, 7H); 4.28-2.90 (m, 10H); 2.69-1.83 (m, 8H)

### Compound 89: 4-[5′,11′-Dihydrospiro[cyclopentane-1,10′-dibenzo[a,d]cyclohepten]-3-yl(methyl)amino]-2,2-dimethylbutanoic acid (diastereoisomer 1)

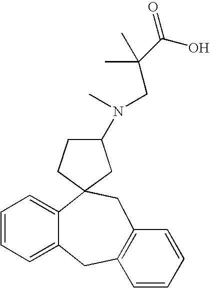

In a 100 mL round-bottomed flask was added methyl 4-[5′,11′-dihydrospiro[cyclopentane-1,10′-dibenzo[a,d]cyclohepten]-3-yl(methyl)amino]-2,2-dimethylbutanoate (diastereoisomer 1, Intermediate 42, 208 mg, 0.513 mmol) and KOH (155 mg 2.051 mmol) in methanol (20 ml) and water (10 ml) to give a colorless suspension. After stirring overnight the reaction was not complete so KOH (28 mg 1 eq.) was added and the reaction mixture heated at 100° C. for 4 hrs. MS monitor showed that the reaction was complete. The solvents were removed to give the crude which was purified by HLB 6 g cartridge to give the title compound (200 mg);

UPLC/MS RT=0.64; m/z (ES): 392.12 [M+H]⁺; ¹H NMR (400 MHz, CHLOROFORM-d) d ppm 7.62-7.55 (m, 1H); 7.24-7.01 (m, 7H); 4.27-3.93 (m, 2H); 3.8-3.62 (m, 1H); 3.35 (s, 2H); 3.26-3.05 (m, 2H); 2.80-2.71 (m, 5H); 2.36-2.82 (m, 6H); 1.29 (s, 3H); 1.28 (s, 3H).

### Compound 90: Methyl 4-(5′,11′-dihydrospiro[cyclopentane-1,10′-dibenzo[a,d]cyclohepten]-3-yl)-2-piperazinecarboxylate

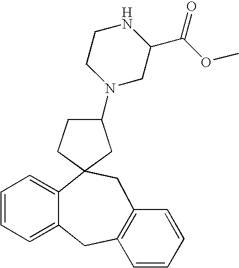

To a solution of 2-methyl 1-(phenylmethyl) 4-(5′,11′-dihydrospiro[cyclopentane-1,10′-dibenzo[a,d]cyclohepten]-3-yl)-1,2-piperazinedicarboxylate (Intermediate 38, 106 mg, 0.202 mmol) in ethanol (15 ml) was added Pd/C (215 mg, 0.202 mmol) and AcOH (0.013 ml, 0.222 mmol). The mixture was stirred at room temperature under hydrogen atmosphere for 2.5 h. Then the palladium was filtered out. Evaporation of solvent gave crude compound (60 mg) which was purified by —NH₂ 5 g cartridge (eluent EtOAc/cycloexane gradient from 1:9 to 3:7) to give the title compound (34 mg, 0.087 mmol) as a mixture of two diastereoisomeric racemates.

UPLC/MS RT=0.68; m/z (ES): 391.23 [M+H]⁺

This isomeric mixture was submitted for chiral HPLC separation (Preparative chromatographic conditions: Column: Chiralcel OD-H, Mobile phase: n-Hexane/Ethanol 70/30% v/v, Flow rate: 13 mL/min; UV: 215 nm) to give 2 singol major isomers:

### Compound 91: Methyl 4-(5′,11′-dihydrospiro[cyclopentane-1,10′-dibenzo[a,d]cyclohepten]-3-yl)-2-piperazinecarboxylate (isomer 1)

retention time=7.5 mins (6 mg);

¹H NMR (400 MHz, CHLOROFORM-d) d ppm 7.40-6.90 (m, 8H); 4.47-3.90 (m, 2H); 3.75 (s, 3H); 3.70-3.57 (m, 1H); 3.39-2.74 (m, 6H); 2.45-1.5 (m, 10H).

### Compound 92: Methyl 4-(5′,11′-dihydrospiro[cyclopentane-1,10′-dibenzo[a,d]cyclohepten]-3-yl)-2-piperazinecarboxylate (isomer 2)

retention time=9.54 mins (4.6 mg);

¹H NMR (400 MHz, CHLOROFORM-d) d ppm 7.40-6.90 (m, 8H); 4.35-3.90 (m, 2H); 3.77 (s, 3H); 3.70-3.60 (m, 1H); 3.29-2.80 (m, 6H); 2.75-1.60 (m, 10H).

### Compound 93: 4-(5′,11′-Dihydrospiro[cyclopentane-1,10′-dibenzo[a,d]cyclohepten]-3-yl)-2-piperazinecarboxylic acid (isomer 1)

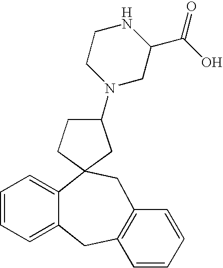

In a round-bottomed flask, methyl 4-(5′,11′-dihydrospiro[cyclopentane-1,10′-dibenzo[a,d]cyclohepten]-3-yl)-2-piperazinecarboxylate (isomer 1,_Compound 91, 6 mg, 0.015 mmol) was dissolved in methanol (1.5 ml) and water (0.3 ml). Then LiOH (1.840 mg, 0.077 mmol) was added. The reaction mixture was left stirring at room temperature overnight. MeOH was evaporated under vacuum and the crude was acidified with 3N HCl checking the pH of the solution (until pH 1) put over HLB Cartridge 1 g. The elution was done first with water and secondly with MeOH. The desired product was checked by TLC using isopropanol:NH₃ as eluente, 70:30. Evaporation of MeOH afforded the title compound (6 mg); m/z (ES): 377.0[M+H]⁺; ¹H NMR (400 MHz, METHANOL-d) d ppm 7.50-7.00 (m, 8H); 4.80-5.10 (m, 1H); 4.76-4.63 (m, 1H); 4.28-4.13 (m, 2H); 4.13-4.01 (m, 1H); 3.96-3.62 (m, 4H); 3.56-3.25 (m, 3H); 2.60-2.13 (m, 5H); 2.11-1.92 (m, 1H).

### Compound 94: 4-(5′,11′-Dihydrospiro[cyclopentane-1,10′-dibenzo[a,d]cyclohepten]-3-yl)-2-piperazinecarboxylic acid (isomer 2)

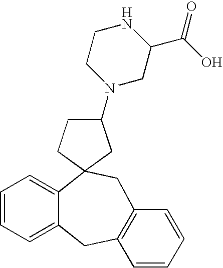

In a round-bottomed flask, methyl 4-(5′,11′-dihydrospiro[cyclopentane-1,10′-dibenzo[a,d]cyclohepten]-3-yl)-2-piperazinecarboxylate (isomer 2, Compound 92, 4.6 mg, 0.012 mmol) was dissolved in methanol (1.5 ml) and water (0.3 ml). Then LiOH (1.410 mg, 0.059 mmol) was added. The reaction mixture was left under stirring at room temperature overnight. MeOH was evaporated under vacuum and the crude was acidified with 3N HCl checking the pH of the solution (until pH 1) put over HLB Cartridge 1 g. The elution was done firstly with water and secondly with MeOH. The desired product was checked in TLC using isopropanol:NH₃ as eluente, 70:30. Evaporation of MeOH afforded the title compound (4.5 mg); m/z (ES): 377.0[M+H]⁺; ¹H NMR (400 MHz, METHANOL-d) d ppm 7.39-6.95 (m, 8H); 4.25-4.14 (m, 1H); 4.14-4.02 (m, 1H); 3.71-3.81 (m, 1H); 3.62-3.53 (m, 1H); 3.45-3.30 (m, 1H); 3.30-3.09 (m, 4H); 3.08-2.95 (m, 1H); 2.54-2.38 (m, 2H); 2.05-2.25 (m, 3H); 2.04-1.89 (m, 3H).

### Compound 95: Methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (diastereomeric mixture 1)

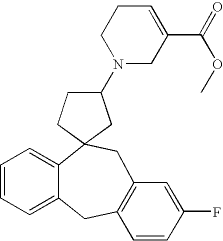

In a 10 mL round-bottomed vial 2′-fluoro-5′,11′-dihydro-3H-spiro[cyclopentane-1,10′-dibenzo[a,d]cyclohepten]-3-one (enantiomer 1) (Intermediate 36, 70 mg, 0.250 mmol), Guvacine methyl ester hydrochloride (53.2 mg, 0.3 mmol) and DIPEA (0.052 mL, 0.3 mmol) were dissolved in DCE (2.5 mL). After 10 min of stirring at room temperature acetic acid (0.028 mL, 0.499 mmol) and sodium triacetoxyborohydride (106 mg, 0.499 mmol) were added. The reaction was stirred at room temperature overnight. The reaction mixture was diluted with DCM (5 ml) and washed with saturated solution of NaHCO₃, brine and then the organic phase was separated and solvent removed under vacuum. The crude compound was submitted for purification through a SCX cartridge eluting with DCM, MeOH, and NH₃ in MeOH 2M. Solvent was evaporated affording the title compound (91 mg) as a mixture of two diastereoisomers.

The mixture of diastereoisomers was submitted for chiral HPLC purification, (Preparative chromatographic conditions: Chiralcel OD-H (25×0.46 cm) Mobile phase: n-Hexane/2-Propanol 88/12% v/v Flow rate: 1.0 ml/min DAD: 210-340 nm CD: 225 nm). The separation was not successful! and the collected fractions afforded a mixture of two diastereoisomers (35 mg, ratio 30.65/69.35).

UPLC/MS Rf=0.81; m/z (ES): 406.26 [M+H]⁺.

### Compound 96: Methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (diastereomeric mixture 2)

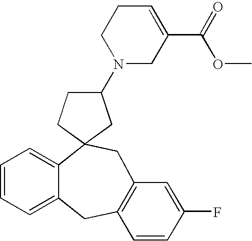

In a 10 mL round-bottomed vial 2′-fluoro-5′,11′-dihydro-3H-spiro[cyclopentane-1,10′-dibenzo[a,d]cyclohepten]-3-one (enantiomer 2) (Intermediate 37, 70 mg, 0.250 mmol), Guvacine methyl ester hydrochloride (53.2 mg, 0.3 mmol) and DIPEA (0.052 mL, 0.3 mmol) were dissolved in DCE (2.5 mL). After 10 min of stirring at room temperature acetic acid (0.028 mL, 0.499 mmol) and sodium triacetoxyborohydride (106 mg, 0.499 mmol) were added. The reaction was stirred at room temperature overnight. The reaction mixture was diluted with DCM (5 ml) and washed with saturated solution of NaHCO₃, brine and then the organic phase was separated and solvent was removed under vacuum. The crude compound was submitted for purification through a SCX cartridge eluting with DCM, MeOH, and NH₃ in MeOH 2M. Solvent was evaporated and the title compound was obtained as mixture of two diastereoisomers (91 mg, ratio 73.61/26.39).

The mixture of diastereoisomers was submitted for chiral HPLC purification (Preparative chromatographic conditions: Chiralcel OD-H (25×0.46 cm); Mobile phase: n-Hexane/2-Propanol 85/15% v/v; Flow rate: 1.0 ml/min; DAD: 210-340 nm; CD: 225 nm), to give:

### Compound 97: Methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (isomer 2)

retention time 7.65 mins (44 mg)

UPLC/MS Rf=0.67; m/z (ES): 406.26 [M+H]⁺.

¹H NMR (400 MHz, CHLOROFORM-d) ppm 1.72-1.99 [m, 4H], 1.99-2.12 [m, 2H], 2.26-2.38 [m, 2H], 2.48-2.60 [m, 2H], 3.00-3.16 [m, 2H], 3.17-3.44 [m, 3H], 3.64-3.69 [s, 3H], 3.94-4.03 [d, 1H], 4.17-4.22 [d, 1H], 6.90-6.96 [td, 1H], 6.94-6.97 [m, 1H], 6.99-7.04 [td, 1H], 7.08-7.13 [m, 2H], 7.15-7.21 [td, 1H], 7.23-7.28 [dd, 1H], 7.28-7.32 [d, 1H].

### Compound 98: Methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (isomer 4)

retention time 14.11 mins (9 mg)

### Compound 99: 1-(2′-Fluoro-5′,11′-dihydrospiro[cyclopentane-1,10′-dibenzo[a,d]cyclohepten]-3-yl)-1,2,5,6-tetrahydro-3-pyridinecarboxylic acid (diastereomeric mixture 1)

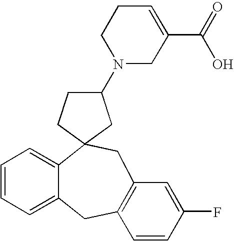

To a suspension of methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (diastereomeric mixture 1, Compound 95, 34 mg, 0.084 mmol) in MeOH/H₂O (2.7 ml, 2/1 v/v), LiOH (10.04 mg, 0.42 mmol) was added and the mixture stirred at 40° C. for 6 h. MeOH was removed and aqueous solution was neutralised with HCl (1M). Mixture was submitted for purification through C18 cartridge, eluting with water and then MeOH. Collected fractions afforded the title compound (25 mg).

UPLC/MS RT=0.63; m/z (ES): 392.18 [M+H]⁺.

### Compound 100: 1-(2′-Fluoro-5′,11′-dihydrospiro[cyclopentane-1,10′-dibenzo[a,d]cyclohepten]-3-yl)-1,2,5,6-tetrahydro-3-pyridinecarboxylic acid (isomer 2)

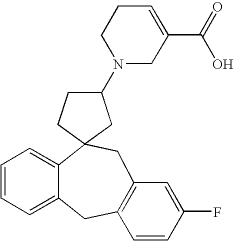

To a suspension of methyl 1-(2′-fluoro-5′,11′-dihydrospiro[cyclopentane-1,10′-dibenzo[a,d]cyclohepten]-3-yl)-1,2,5,6-tetrahydro-3-pyridinecarboxylate (major diastereoisomer 2, Compound 97, 40 mg, 0.099 mmol) in MeOH/H₂O (3.2 ml, 2/1 v/v), LiOH (11.81 mg, 0.49 mmol) was added and the mixture stirred at 40° C. for 6 h. MeOH was removed and aqueous solution was neutralised with HCl (1M). The mixture was submitted for purification through C18 cartridge, eluting with water and then MeOH. Collected fractions afforded the title compound (32 mg);

UPLC/MS RT=0.63; m/z (ES): 392.18 [M+H]⁺; ¹H NMR (400 MHz, DMSO-d) ppm 12.68-12.04 [br. S., 1H], 7.32-7.28 [d, 1H], 7.28-7.23 [dd, 1H], 7.21-7.16 [td, 1H], 7.13-7.08 [m, 2H], 7.13-7.08 [m, 2H], 7.04-6.99 [td, 1H], 6.96-6.89 [td, 1H], 6.90-6.85 [m, 1H], 4.26-4.16 [d, 1H], 4.04-3.93 [d, 1H], 3.40-3.16 [m, 1H], 3.28-3.16 [m, 2H], 3.15-2.99 [m, 2H], 2.60-2.51 [m, 2H], 2.31-2.24 [m, 2H], 2.11-1.98 [m, 2H], 1.99-1.70 [m, 4H].

### Compound 101: Ethyl 1-(2′-chloro-11′-oxo-11′H-spiro[cyclopentane-1,10′-dibenzo[b,f]oxepin]-3-yl)-4-piperidinecarboxylate

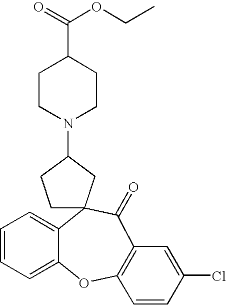

2′-Chloro-3H,11′H-spiro[cyclopentane-1,10′-dibenzo[b,f]oxepin]-3,11′-dione (Intermediate 19, 100 mg, 0.319 mmol), ethyl-4-piperidine carboxylate (100 mg, 0.638 mmol) and glacial acetic acid (150 μl, 2.2 mmol) were dissolved in 1,2 dichloroethane (20 ml) and stirred at room temperature for 1.5 hours. Sodium triacetoxyborohydride (270 mg, 1.2 mmol) was added. After 1 day 1 ml of reaction mixture was taken for another reaction. After 30 hours additional ethyl-4-piperidine carboxylate (50 mg) and sodium triacetoxyborohydride (135 mg) were added. Stirring was continued for another 2 h and then the reaction mixture was washed with NaHCO₃(sat.) and the organic layer was evaporated giving a crude oil (740 mg). The oil was purified using flash chromatography on Si cartridge (5 g). Washing with 8 ml nHex:EtOAc=4:1 gave the title compound (70 mg) as an oil.

HPLC-MS m/z: 454.0 [M+1]+; Rt: 12.97 min.

¹H NMR (CDCl3): δ 1.18-1.28 (t, 3H), 1.52-2.06 (m, 8H), 2.1-2.32 (m, 3H), 2.59-2.71 (m, 2H), 2.8-2.86 (m, 3H), 4.06-4.13 (q, 2H), 7.19-7.33 (m, 5H), 7.44-7.48 (dd, 1H), 7.96-7.97 (d, 1H);

### Compound 102: 1-(2′-Chloro-11′-oxo-11′H-spiro[cyclopentane-1,10′-dibenzo[b,f]oxepin]-3-yl)-4-piperidinecarboxylic acid

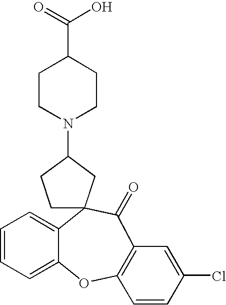

Ethyl 1-(2′-chloro-11′-oxo-11′H-spiro[cyclopentane-1,10′-dibenzo[b,f]oxepin]-3-yl)-4-piperidinecarboxylate (Compound 101, 40 mg, 0.088 mmol) and KOH (19 mg, 0.339 mmol) were dissolved in EtOH (4 ml) and water (1 ml). The reaction mixture was then heated in a microwave reactor for 1 hour at 70° C. and 250 W. The solvent was then evaporated and the residue was dissolved in 2N HCl. Some precipitate appeared. The suspension was purified by a HLB Oasis column (1 g) eluting first with water (10 ml) and then with MeOH (10 ml). The MeOH fraction was evaporated to give the title compound (26.3 mg) as an oil.

HPLC-MS m/z: 426.0 [M+1]+; Rt: 12.17 min.

### Compound 103: Ethyl 1-(2′-chloro-11′-oxo-11′H-spiro[cyclopentane-1,10′-dibenzo[b,f]oxepin]-3-yl)-3-piperidinecarboxylate

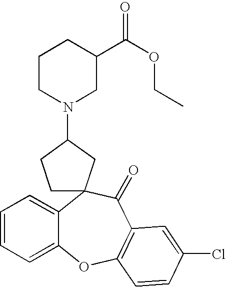

2′-Chloro-3H,11′H-spiro[cyclopentane-1,10′-dibenzo[b,f]oxepin]-3,11′-dione (Intermediate 19, 116 mg, 0.37 mmol) and ethyl-3-piperidine carboxylate (116 mg, 0.74 mmol) were dissolved in 1,2 dichloroethane (20 ml) and stirred at room temperature for 1 hour then sodium triacetoxyborohydride (310 mg, 1.4 mmol) was added and stirred for another 4 h. After that glacial acetic acid (150 μl, 2.5 mmol). After 72 hours additional ethyl-4-piperidine carboxylate (58 mg) and sodium triacetoxyborohydride (160 mg) were added. Stirring was continued for another 4 h. The mixture was then washed with NaHCO₃(sat.) and the organic layer was evaporated giving an oil. The oil was purified using flash chromatography on Supelco prepacked column (10 g). The oil was dissolved in the smallest amount possible of system DCM:MeOH:NH₃=99:1:0.1 and put on the column. It was then washed with DCM:MeOH:NH₃=99:1:0.1 (13 ml), DCM:MeOH:NH₃=97:3:0.1(2 ml), DCM:MeOH:NH₃=95:5:0.1 (5 ml) and DCM:MeOH:NH₃=93:7:0.1 (17 ml). The last two fractions were merged and evaporated giving the title compound (90 mg) as an oil.

HPLC-MS m/z: 454.2 [M+1]+; Rt: 13.18 min.

¹H NMR (CDCl3): δ 1.19-1.23 (q, 3H), 1.33-1.41 (m, 1H), 1.41-1.66 (m, 4H), 1.89-2.18 (m, 2H), 2.31 (t, 1H), 2.42-2.84 (m, 5H), 2.97 (s,1H) 7.20-7.33 (m, 5H), 7.45-7.49 (dd, 1H), 7.96-7.97 (m, 1H);

### Compound 104: 1-(2′-Chloro-11′-oxo-11′H-spiro[cyclopentane-1,10′-dibenzo[b,f]oxepin]-3-yl)-3-piperidinecarboxylic acid-hydrochloride

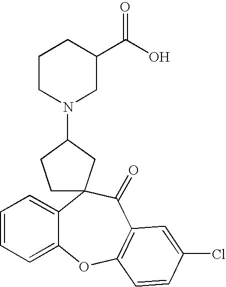

Ethyl 1-(2′-chloro-11′-oxo-11′H-spiro[cyclopentane-1,10′-dibenzo[b,f]oxepin]-3-yl)-3-piperidinecarboxylate (Compound 103, 40 mg, 0.088 mmol) and KOH (19 mg, 0.339 mmol) were dissolved in EtOH (4 ml) and water (1 ml). The reaction mixture was then heated in a microwave reactor for 1 hour at 70° C. and 250 W. The solvent was then evaporated and the residue was dissolved in 2N HCl. Amine precipitated in the form of HCl salt which was separated by centrifugation and washed with water (3×6 ml). Finally it was dried at 40° C. and low vacuum (1 mmHg) for 4 h. The title compound was obtained (21.3 mg) as a yellow powder.

HPLC-MS m/z: [426.2+1]+; Rt: 11.66 min.

### Compound 105: Ethyl 1-(11′H-spiro[cyclopentane-1,10′-dibenzo[b,f]oxepin]-3-yl)-4-piperidinecarboxylate

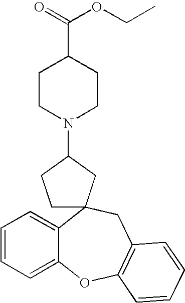

3H,11′H-spiro[cyclopentane-1,10′-dibenzo[b,f]oxepin]-3-one (Intermediate 20, 40 mg, 0.15 mmol), ethyl-4-piperidine carboxylate (47 mg, 0.3 mmol) and glacial acetic acid (100 μl, 1.5 mmol) were dissolved in 1,2 dichloroethane (5 ml) and stirred at room temperature for 1.5 hours then sodium triacetoxyborohydride (135 mg, 0.6 mmol) was added. After 24 h the reaction mixture was washed with NaHCO₃(sat.) and the organic layer was evaporated after drying over Na₂SO₄/MgSO₄ giving a crude oil (44.2 mg). The oil was dissolved in a small amount of system DCM:MeOH:NH₃=99:1:0.1 and put on a Supelco prepacked silica column (2 g). Separation was made with a solvent gradient which included DCM:MeOH:NH₃=99:1:0.1, DCM:MeOH:NH₃=98:2:0.1, DCM:MeOH:NH₃=97:3:0.1, DCM:MeOH:NH₃=95:5:0.1 and DCM:MeOH:NH₃=93:7:0.1 respectively. The title compound was obtained (12.5 mg) as an oil.

HPLC-MS m/z: [406.2+1]+; Rt: 12.63 min.

### Compound 106: 1-(11′H-Spiro[cyclopentane-1,10′-dibenzo[b,f]oxepin]-3-yl)-4-piperidinecarboxylic acid

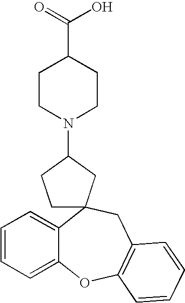

Ethyl 1-(11′H-spiro[cyclopentane-1,10′-dibenzo[b,f]oxepin]-3-yl)-4-piperidinecarboxylate (Compound 105, 12 mg, 0.030 mmol) and KOH (6.6 mg, 0.12 mmol) were dissolved in EtOH (4 ml) and water (1 ml). The reaction mixture was then heated in a microwave reactor for 1.5 hour at 70° C. and 250 W. After that the solvent was evaporated and the residue was dissolved in 2N HCl. Amine precipitated in the form of HCl salt which was separated by centrifugation and washed with water (3×6 ml). Finally it was dried at 40° C. and low vacuum (1 mmHg) for 4 h. The title compound was obtained (7 mg) as a yellow powder.

HPLC-MS m/z: 378.1 [M+1]+; Rt: 11.24 min.

### Compound 107: Ethyl (3R)-1-(11′H-spiro[cyclopentane-1,10′-dibenzo[b,f]oxepin]-3-yl)-3-piperidinecarboxylate

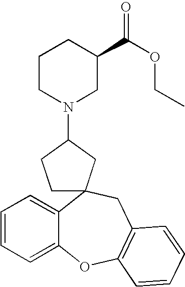

3H,11′H-spiro[cyclopentane-1,10′-dibenzo[b,f]oxepin]-3-one (Intermediate 20, 40 mg, 0.15 mmol), ethyl-(3R)-piperidine carboxylate-L-tartarate salt (70 mg, 0.3 mmol), diisopropylethylene amine (38.7 mg, 0.3 mmol) and glacial acetic acid (100 μl, 1.5 mmol) were dissolved in 1,2 dichloroethane (5 ml) and stirred at room temperature for 1.5 hours then sodium triacetoxyborohydride (135 mg, 0.6 mmol) was added. After 24 h the reaction mixture was washed with NaHCO₃(sat.) and the organic layer was evaporated after drying over Na₂SO₄/MgSO₄ giving to give a crude (44.2 mg) oil. The oil was dissolved in a small amount of system DCM:MeOH:NH₃=99:1:0.1 and put on a Supelco prepacked silica column (2 g). Separation was made with a solvent gradient which included DCM:MeOH:NH₃=99:1:0.1, DCM:MeOH:NH₃=98:2:0.1, DCM:MeOH:NH₃=97:3:0.1, DCM:MeOH:NH₃=95:5:0.1 and DCM:MeOH:NH₃=93:7:0.1 respectively. The title compound was obtained (15 mg) as an oil.

HPLC-MS m/z: [406.2+1]+; Rt: 12.13 min. purity: HPLC-UV: 84.55%; HPLC-MS: 81.04%;

### Compound 108: (3R)-1-(11′H-spiro[cyclopentane-1,10′-dibenzo[b,f]oxepine]-3-yl)-3-piperidinecarboxylic acid

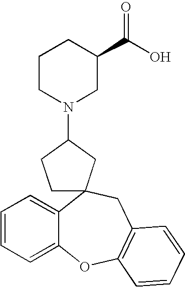

Ethyl (3R)-1-(11′H-spiro[cyclopentane-1,10′-dibenzo[b,f]oxepin]-3-yl)-3-piperidinecarboxylate (Compound 107, 15 mg, 0.036 mmol) and KOH (8 mg, 0.14 mmol) were dissolved in EtOH (4 ml) and water (1 ml). The reaction mixture was heated in a microwave reactor for 1.5 hour at 70° C. and 250 W. After that the solvent was evaporated and the residue was dissolved in 2N HCl. Amine precipitated in the form of HCl salt which was separated by centrifugation and washed with water (3×6 ml). Finally it was dried at 40° C. and low vacuum (1 mmHg) for 4 h. The title compound was obtained (10 mg) as a yellow powder.

HPLC-MS m/z: 378.2[M+1]+; Rt: 11.95 min.

### Compound 109: Methyl 1-(5′,11′-dihydrospiro[cyclopentane-1,10′-dibenzo[a,d]cyclohepten]-3-yl)-4-hydroxy-4-piperidinecarboxylate

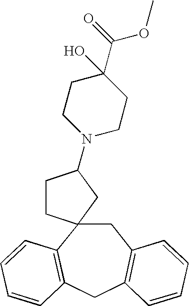

5′,11′-dihydro-3H-spiro[cyclopentane-1,10′-dibenzo[a,d]cyclohepten]-3-one (Intermediate 5, 150 mg, 0.572 mmol) and methyl 4-hydroxy-4-piperidinecarboxylate (190 mg, 1.194 mmol, available from Chemstep) was suspended in methanol (6 mL). Zinc chloride (39.0 mg, 0.286 mmol) was added to this suspension which was then stirred for 2 h. Sodium cyanoborohydride (144 mg, 2.287 mmol) was added and the reaction was left overnight. The reaction was quenched by addition of saturated Na₂CO₃ water solution. The compound was extracted from this mixture using DCM. The combined organic layers were separated and solvent was evaporated to obtain an oil which was treated with diethylether to obtain the title compound (220 mg) as a white solid as a mixture of two diastereoisomeric racemates.

m/z (ES): 406.1 [M+H]⁺

The stereoisomeric mixture was submitted for chiral HPLC purification (Preparative chromatographic conditions: Column=Chiralcel OJ-H (25.0×2.0 cm); Mobile phase=n-Hexane/Ethanol 80/20% v/v; Flow rate=14.0 mL/min; DAD=210-340 nm; CD=225 nm) to give:

### Compound 110: Methyl 1-(5′,11′-dihydrospiro[cyclopentane-1,10′-dibenzo[a,d]cyclohepten]-3-yl)-4-hydroxy-4-piperidinecarboxylate (isomer 1)

retention time=11.64 minutes (84 mg)

¹H NMR (400 MHz, CHLOROFORM-d) δ/ppm 7.31 (d, 1H, J=8 Hz) 7.27-7.10 (m, 6H) 7.05 (t, 1H, J=8 Hz) 4.28 (d, 1H, J=16 hz) 4.01 (d, 1H, J=16 Hz) 3.81 (s, 3H) 3.31 (d, 1H, J=16 Hz) 3.10 (d, 1H, J=16 Hz) 3.06-3.00 (m, 2H) 2.82(d, 1H, J=12 Hz) 2.47-2.39 (m, 2H) 2.25-2.11 (m, 5H) 1.97-1.87 (m, 3H) 1.74-1.66 (m, 2H).

### Compound 111: Methyl 1-(5′,11′-dihydrospiro[cyclopentane-1,10′-dibenzo[a,d]cyclohepten]-3-yl)-4-hydroxy-4-piperidinecarboxylate (isomer 2)

retention time=13.65 minutes (79 mg)

¹H NMR (400 MHz, CHLOROFORM-d) δ/ppm 7.31 (d, 1H, J=8 Hz) 7.26-7.12 (m, 6H) 7.05 (t, 1H, J=8 Hz) 4.28 (d, 1H, J=16 Hz) 4.01 (d, 1H, J=16 Hz) 3.79 (s, 3H) 3.30 (d, 1H, J=12 Hz) 3.14 (d, 1H, J=12 Hz) 3.08-3.00 (m, 2H) 2.82 (d, 1H, J=12 Hz) 2.50-2.42 (m, 2H) 2.25-2.11 (m, 5H) 1.97-1.87 (m, 3H) 1.75-1.66 (m, 2H).

### Compound 112: Methyl 1-(5′,11′-dihydrospiro[cyclopentane-1,10′-dibenzo[a,d]cyclohepten]-3-yl)-4-hydroxy-4-piperidinecarboxylate (isomer 3)

retention time=17.27 minutes (8.8 mg)

¹H NMR (400 MHz, CHLOROFORM-d) δ/ppm 7.47 (d, 1H, J=8 Hz) 7.23-7.12 (m, 6H) 7.05 (t, 1H, J=8 Hz) 4.29 (d, 1H, J=16 Hz) 4.02 (d, 1H, J=16 Hz) 3.81 (s, 3H) 3.51 (s, 1H) 3.24 (d, 1H, J=16 Hz) 3.05 (d, 1H, J=16 Hz) 3.05-2.98 (m, 2H) 2.82 (d, 1H, J=12 Hz) 2.45-2.37 (m, 2H) 2.24-2.13 (m, 5H) 2.05-1.90 (m, 2H) 1.84(m, 1H) 1.73-1.63 (m, 2H).

### Compound 113: Methyl 1-(5′,11′-dihydrospiro[cyclopentane-1,10′-dibenzo[a,d]cyclohepten]-3-yl)-4-hydroxy-4-piperidinecarboxylate (isomer 4)

retention time=18.70 minutes (17.2 mg)

¹H NMR (400 MHz, CHLOROFORM-d) δ/ppm 7.47 (d, 1H, J=8 Hz) 7.23 (d, 1H, J=8 Hz) 7.20-7.12 (m, 5H) 7.05 (t, 1H, J=8 Hz) 4.29 (d, 1H, J=16 Hz) 4.01 (d, 1H, J=16 Hz) 3.81 (s, 3H) 3.51 (s, 1H) 3.24 (d, 1H, J=16 Hz) 3.04 (d, 1H, J=16 Hz) 3.05-2.97 (m, 2H) 2.82 (d, 1H, J=12 Hz) 2.47-2.41 (m, 2H) 2.26-2.15 (m, 5H) 2.10-1.87 (m, 2H) 1.84(m, 1H) 1.72-1.63 (m, 2H).

### Compound 114: 1-(5′,11-dihydrospiro[cyclopentane-1,10′-dibenzo[a,d]cyclohepten]-3-yl)-4-hydroxy-4-piperidinecarboxylic acid (isomer 1)

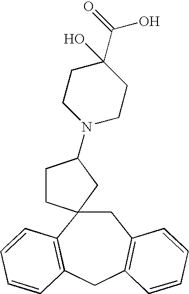

Methyl 1-(5′,11′-dihydrospiro[cyclopentane-1,10′-dibenzo[a,d]cyclohepten]-3-yl)-4-hydroxy-4-piperidinecarboxylate (isomer 1, Compound 110, 84 mg, 0.207 mmol) was dissolved in a mixture of methanol (4.5 ml) and water (2.5 ml). To this solution was added lithium hydroxide (50 mg, 2.088 mmol) and the mixture was stirred overnight (12 h) at room temperature. Reaction was quenched by solvent evaporation. This solid was suspended in water and the pH value was adjusted to 6-7. The resulting solid was washed with water and then triturated in Et₂O to give the title compound (62 mg).

m/z (ES): 392.3 [M+H]⁺

¹H NMR (400 MHz, CHLOROFORM-d) δ/ppm 7.38 (d, 1H, J=8 Hz) 7.28-7.23 (m, 2H) 7.21-7.02 (m, 5H) 4.15-4.10 (m, 2H) 3.75-3.70 (m, 1H) 3.60-3.50 (m, 1H) 3.40-3.20 (m, 2H) 3.20-3.00 (m, 2H) 2.75-2.50 (m, 3H) 2.35-2.33 (m, 1H) 2.25-2.20 (m, 2H) 2.05-1.75(m, 7H).

### Compound 115: 1-(5′,11′-Dihydrospiro[cyclopentane-1,10′-dibenzo[a,d]cyclohepten]-3-yl)-4-hydroxy-4-piperidinecarboxylic acid (isomer 2)

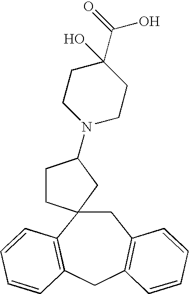

Methyl 1-(5′,11′-dihydrospiro[cyclopentane-1,10′-dibenzo[a,d]cyclohepten]-3-yl)-4-hydroxy-4-piperidinecarboxylate (isomer 2, Compound 111, 79 mg, 0.195 mmol) was dissolved in methanol (4.5 ml). In the solution were put water (2.5 ml) and lithium hydroxide (30 mg, 1.253 mmol). The reaction was left overnight on room temperature. Solvent was then evaporated to give a solid that was suspended in water; pH value was adjusted to 6-7. The resulting solid was washed with water and then triturated in Et₂O to give the title compound (15 mg).

m/z (ES): 392.3 [M+H]⁺

¹H NMR (400 MHz, CHLOROFORM-d) δ/ppm 7.36 (m, 1H) 7.30-7.24 (m, 1H) 7.21-7.02 (m, 6H) 4.25-4.05 (m, 2H) 3.70 (m, 1H) 3.60 (m, 1H) 3.30-3.24 (m, 2H) 3.20-2.95 (m, 2H) 2.80-2.50 (m, 4H) 2.35-2.33 (m, 1H) 2.35-2.30 (m, 1H) 2.00-1.60 (m, 7H).

### Compound 116: 1-(5′,11′-Dihydrospiro[cyclopentane-1,10′-dibenzo[a,d]cyclohepten]-3-yl)-3-piperidinecarboxamide (isomer 2)

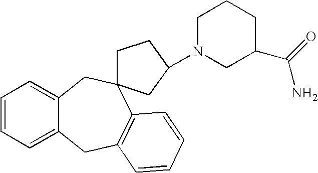

To a solution of 1-(5′,11′-dihydrospiro[cyclopentane-1,10′-dibenzo[a,d]cyclohepten]-3-yl)-3-piperidinecarboxylic acid (isomer 2, Compound 1230 mg, 0.080 mmol) in DMF (1 ml), under nitrogen and at room temperature, were added DIPEA (28 μl, 0.160 mmol) and TBTU (30.8 mg, 0.096 mmol). The reaction mixture was stirred for 30 min and HMDS (20.23 μl, 0.096 mmol) was then added. After 16 hrs the reaction was quenched with NaHCO₃ (sat solution), extracted with DCM, the combined organic layers were dried (Na₂SO₄) and concentrated in vacuo. The crude product was purified by flash chromatography on silica gel, eluting with a gradient of MeOH in DCM (from 0 to 5%) to afford the title compound (22 mg);

MS; m/z (ES): 375.2 [M+H]⁺; ¹H NMR (400 MHz, CHLOROFORM-d) d ppm 7.02-7.30 (m, 9H) 5.46 (bs, 1H) 4.20 (d, 1H) 4.08 (d, 1H) 3.26 (d, 1H) 3.10-3.20 (m, 2H) 2.60-2.95 (m, 4H) 1.70-2.30 (m, 11H).

### Compound 117: Ethyl-3-(5′,11′-dihydrospiro[cyclopentane-1,10′-dibenzo[a,d]cyclohepten]-3-yl)-3-azabicyclo[3.1.0]hexane-6-carboxylate (exo)

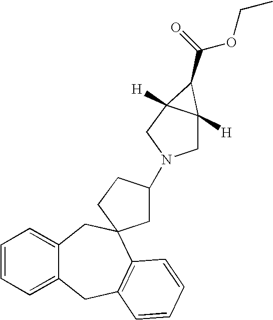

To a solution of ethyl-3-azabicyclo[3.1.0]hexane-6-carboxylate (for preparation see WO 2007/055093) (exo, 116 mg, 0.747 mmol) and 5′,11′-dihydro-3H-spiro[cyclopentane-1,10′-dibenzo[a,d]cyclohepten]-3-one (Intermediate 5, 196 mg, 0.747 mmol) in dry DCE (4 ml) under nitrogen, was added a drop of AcOH and the resulting mixture was stirred at room temperature for 30 min. Sodium triacetoxyborohydride (190 mg, 0.897 mmol) was then added and the resulting reaction mixture was stirred for 16 hrs, quenched with NaHCO₃ (saturated aqueous solution) and extracted with DCM. The organic layers were combined, dried (Na₂SO₄) and concentrated in vacuo. The crude product was purified by flash chromatography on silica gel (25 g) eluting with a gradient from DCM/methanol from 100/0 to 92/2 affording a mixture of two diastereoisomeric racemates of the title compound (266 mg) as a white solid.

The isomeric mixture was submitted for chiral HPLC purification (Preparative chromatographic conditions: Column: Chiralcel OJ-H; Mobile phase: n-Hexane/(Ethanol+0.1% isopropylamine) 80/20% v/v; Flow rate: 0.8 mL/min; UV: 215 nm) to give one single isomer (Compound 118) and a mixture.

### Compound 118: Ethyl-3-(5′,11′-dihydrospiro[cyclopentane-1,10′-dibenzo[a,d]cyclohepten]-3-yl-3-azabicyclo[3.1.0]hexane-6-carboxylate (exo) (isomer 1)

retention time=11.3 mins (69 mg).

¹H NMR (400 MHz, CHLOROFORM-d) d ppm 7.0-7.3 (m, 8H) 4.20 (d, 1H) 4.16 (q, 2H) 4.06 (d, 1H) 3.08-3.24 (m, 4H) 2.97 (m, 1H) 2.45 (m, 1H) 2.36 (m, 1H) 2.13 (m, 1H) 1.80-2.07 (m, 8H) 1.28 (t, 3H).

The mixture was submitted to further chiral HPLC purification (Preparative chromatographic conditions: Column: Chiralcel OJ-H; Mobile phase: n-Hexane/(Ethanol+0.1% isopropylamine) 80/20% v/v; Flow rate: 0.8 mL/min; UV: 215 nm) to give Compound 119 as single isomer.

### Compound 119: Ethyl-3-(5′,11′-dihydrospiro[cyclopentane-1,10′-dibenzo[a,d]cyclohepten]-3-yl)-3-azabicyclo[3.1.0]hexane-6-carboxylate (exo) (isomer 2)

retention time=13.2 mins (35 mg).

¹H NMR (400 MHz, CHLOROFORM-d) d ppm 7.0-7.3 (m, 8H) 4.20 (d, 1H) 4.16 (q, 2H) 4.06 (d, 1H) 3.08-3.24 (m, 4H) 2.97 (m, 1H) 2.45 (m, 1H) 2.36 (m, 1H) 2.13 (m, 1H) 1.80-2.07 (m, 8H) 1.28 (t, 3H).

### Compound 120: 3-(5′,11′-Dihydrospiro[cyclopentane-1,10′-dibenzo[a,d]cyclohepten]-3-yl)-3-azabicyclo[3.1.0]hexane-6-carboxylic acid (exo) (isomer 1)

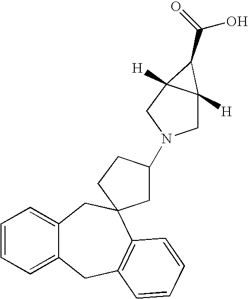

To a solution of ethyl-3-(5′,11′-dihydrospiro[cyclopentane-1,10′-dibenzo[a,d]cyclohepten]-3-yl)-3-azabicyclo[3.1.0]hexane-6-carboxylate (isomer 1, Compound 118, 69 mg, 0.172 mmol) in methanol (3 ml) and water (1 ml), was added LiOH (20.58 mg, 0.859 mmol) and the mixture was refluxed for 4 hours. The solvent was concentrated at reduced pressure and the residue was dissolved in water and neutralized with HCl (1M). This mixture was purified by C18 column (10 g) to give the title compound (25 mg) as a white solid.

MS; m/z (ES): 374.0 [M+H]⁺;

¹H NMR (400 MHz, CHLOROFORM-d) d ppm 7.0-7.3 (m, 8H) 4.18 (d, 1H) 4.07 (d, 1H) 2.95-3.35 (m, 5H) 2.56 (m, 1H) 2.47 (m, 1H) 2.20 (m, 1H) 1.75-2.13 (m, 8H).

### Compound 121: 3-(5′,11′-Dihydrospiro[cyclopentane-1,10′-dibenzo[a,d]cyclohepten]-3-yl)-3-azabicyclo[3.1.0]hexane-6-carboxylic acid hydrochloride (exo) (isomer 2)

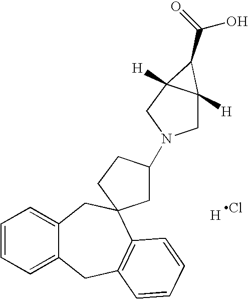

To a solution of ethyl-3-(5′,11′-dihydrospiro[cyclopentane-1,10′-dibenzo[a,d]cyclohepten]-3-yl)-3-azabicyclo[3.1.0]hexane-6-carboxylate (isomer 2, Compound 119, 35 mg, 0.087 mmol) in methanol (3 ml) and water (1 ml), was added LiOH (10.44 mg, 0.436 mmol) and the mixture was refluxed for 4 hours. The solvent was concentrated at reduced pressure; the residue was dissolved in water and acidified with HCl (1M). This mixture was purified by C18 column (10 g) to give the title compound (25 mg) as a white solid.

MS; m/z (ES): 374.1 [M+H]⁺;

¹H NMR (400 MHz, DMSO-d₆) d ppm 12.51 (bs, 1H) 10.80 (bs, 1H) 7.01-7.50 (m, 8H) 3.95-4.25 (m, 3H) 3.77 (m, 1H) 3.59 (m, 1H) 3.48 (m, 2H) 3.15-3.40 (m, 2H) 2.45-2.65 (m, 1H) 1.96-2.33 (m, 7H) 1.81 (m, 1H).

### Compound 122: [1-(5′,11′-Dihydrospiro[cyclopentane-1,10′-dibenzo[a,d]cyclohepten]-3-yl)-3-pyrrolidinyl]acetic acid (diastereomeric mixture 3)

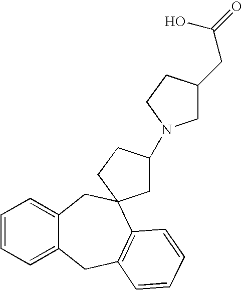

To a solution of methyl[1-(5′,11′-dihydrospiro[cyclopentane-1,10′-dibenzo[a,d]cyclohepten]-3-yl)-3-pyrrolidinyl]acetate (diastereomeric mixture 3, Intermediate 44, 24 mg, 0.062 mmol) in methanol (3 ml) and water (1.0 ml), was added LiOH (7.38 mg, 0.308 mmol) and the mixture was refluxed for 4 hours. The solvent was concentrated at reduced pressure and the residue was dissolved in water and neutralized with HCl (1M). This mixture was purified by C18 column (5 g) to give the title compound (20 mg) as beige solid;

MS; m/z (ES): 376.1 [M+H]⁺; ¹H NMR (400 MHz, DMSO-d₆) d ppm 6.99-7.29 (m, 8H) 4.18 (d, 1H) 4.02 (d, 1H) 3.21 (d, 1H) 3.08 (d, 1H) 2.93 (m, 1H) 2.78 (m, 1H) 2.15-2.60 (m, 7H) 1.70-2.05 (m, 6H) 1.37 (m, 1H).

### Compound 123: [1-(5′,11′-Dihydrospiro[cyclopentane-1,10′-dibenzo[a,d]cyclohepten]-3-yl)-3-pyrrolidinyl]acetic acid (isomer 2)

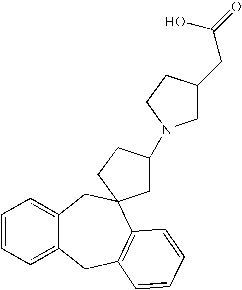

To a solution of methyl[1-(5′,11′-dihydrospiro[cyclopentane-1,10′-dibenzo[a,d]cyclohepten]-3-yl)-3-pyrrolidinyl]acetate (isomer 2, Intermediate 45, 28 mg, 0.072 mmol) in methanol (3 ml) and water (1.000 ml), was added LiOH (8.61 mg, 0.359 mmol) and the mixture was refluxed for 4 hours. The solvent was concentrated at reduced pressure; the residue was dissolved in water and neutralized with HCl (1M). This mixture was purified by C18 column (5 g) to give the title compound (23 mg) as beige solid.

MS; m/z (ES): 376.1 [M+H]⁺;

¹H NMR (400 MHz, DMSO-d₆) d ppm 6.99-7.29 (m, 8H) 4.18 (d, 1H) 4.02 (d, 1H) 3.21 (d, 1H) 3.08 (d, 1H) 2.93 (m, 1H) 2.78 (m, 1H) 2.32-2.60 (m, 6H) 2.20 (m, 1H) 1.70-2.05 (m, 6H) 1.37 (m, 1H).

### Compound 124: Ethyl 1-(5′,11′-dihydrospiro[cyclopentane-1,10′-dibenzo[a,d]cyclohepten]-3-yl)-4-methyl-4-piperidinecarboxylate

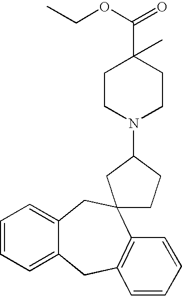

To a mixture of 5′,11′-dihydro-3H-spiro[cyclopentane-1,10′-dibenzo[a,d]cyclohepten]-3-one (Intermediate 5, 150 mg, 0.572 mmol) and ethyl 4-methyl-4-piperidinecarboxylate (see U.S. Pat. No. 6,720,338 example 532C) (147 mg, 0.858 mmol) in dry DCE (3 ml) under nitrogen, was added a drop of AcOH and stirred at room temperature for 30 min. Sodium triacetoxyborohydride (242 mg, 1.144 mmol) was then added and the resulting reaction mixture was stirred for 3 days and further sodium triacetoxyborohydride (140 mg) was added. The mixture was left to stir at room temperature for 24 hrs, quenched with NaHCO₃ (saturated aqueous solution) and extracted with dichloromethane. The organic layers were combined, dried (Na₂SO₄) and concentrated in vacuo. The crude product was purified by flash chromatography on silica gel (25 g) eluting with a gradient from DCM/methanol 100 to 98/2 affording a mixture of two diastereoisomeric racemates of the title compound (166 mg).

The isomeric mixture was submitted for chiral HPLC purification (Preparative chromatographic conditions. Column: Chiralcel OD-H; Mobile phase: n-Hexane/Ethanol 96/4% v/v; Flow rate: 0.8 mL/min; UV: 225 nm), to give a mixture of two isomers (retention time=6.8 min) and a single isomer (retention time=8.6, Compound 126)

The mixture of isomers was submitted to further chiral HPLC purification (Preparative chromatographic conditions. Column: Whelk O1 (R,R); Mobile phase: n-Hexane/2-Propanol 95/5% v/v; Flow rate: 1.0 mL/min; UV: 225 nm), to give Compound 125 as single isomer.

### Compound 125: Ethyl 1-(5′,11′-dihydrospiro[cyclopentane-1,10′-dibenzo[a,d]cyclohepten]-3-yl)-4-methyl-4-piperidinecarboxylate (isomer 1)

retention time (second run)=12.6 mins (55 mg).

MS; m/z (ES): 418.1 [M+H]⁺

¹H NMR (400 MHz, CHLOROFORM-d) d ppm 7.02-7.33 (m, 8H) 4.30 (d, 1H) 4.19 (q, 2H) 3.98 (d, 1H) 3.30 (d, 1H) 3.10 (d, 1H) 2.89 (m, 2H) 2.73 (m, 1H) 2.08-2.23 (m, 6H) 1.75-1.92 (m, 3H) 1.50-1.65 (m, 3H) 1.31 (t, 3H) 1.22 (s, 3H).

### Compound 126: Ethyl 1-(5′,11′-dihydrospiro[cyclopentane-1,10′-dibenzo[a,d]cyclohepten]-3-yl)-4-methyl-4-piperidinecarboxylate (isomer 2)

retention time=8.6 mins_(60 mg).

MS; m/z (ES): 418.15 [M+H]⁺

¹H NMR (400 MHz, CHLOROFORM-d) d ppm 7.02-7.33 (m, 8H) 4.30 (d, 1H) 4.19 (q, 2H) 3.98 (d, 1H) 3.30 (d, 1H) 3.10 (d, 1H) 2.89 (m, 2H) 2.73 (m, 1H) 2.08-2.23 (m, 6H) 1.75-1.92 (m, 3H) 1.50-1.65 (m, 3H) 1.31 (t, 3H) 1.22 (s, 3H).

### Compound 127: 1-(5′,11′-Dihydrospiro[cyclopentane-1,10′-dibenzo[a,d]cyclohepten]-3-yl)-4-methyl-4-piperidinecarboxylic acid (isomer 1)

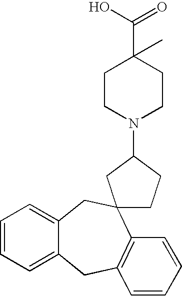

To a solution of ethyl 1-(5′,11′-dihydrospiro[cyclopentane-1,10′-dibenzo[a,d]cyclohepten]-3-yl)-4-methyl-4-piperidinecarboxylate (isomer 1, Compound 125, 55 mg, 0.132 mmol) in methanol (4 ml) and water (1.3 ml), was added LiOH (15.77 mg, 0.659 mmol) and the mixture was refluxed for 4 hours. The solvent was concentrated at reduced pressure, the residue was dissolved in water and neutralized with HCl (1M). This mixture was purified by C18 column (10 g) to give the title compound (30 mg) as a white solid.

MS; m/z (ES): 390.2 [M+H]⁺

¹H NMR (400 MHz, METHANOL-d₄) d ppm 7.04-7.30 (m, 8H) 4.27 (d, 1H) 4.05 (d, 1H) 3.95-4.07 (m, 1H) 3.55-3.65 (m, 1H) 3.38-3.54 (m, 1H) 3.35 (d, 1H) 3.18 (d, 1H) 2.95-3.20 (m, 2H) 1.85-2.50 (m, 8H) 1.55-1.80 (m, 2H) 1.29 (s, 3H).

### Compound 128: 1-(5′,11′-Dihydrospiro[cyclopentane-1,10′-dibenzo[a,d]cyclohepten]-3-yl)-4-methyl-4-piperidinecarboxylic acid (isomer 2)

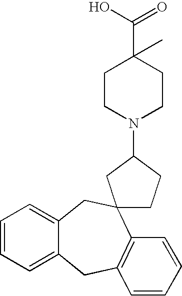

To a solution of ethyl 1-(5′,11′-dihydrospiro[cyclopentane-1,10′-dibenzo[a,d]cyclohepten]-3-yl)-4-methyl-4-piperidinecarboxylate (isomer 2, Compound 126, 60 mg, 0.144 mmol) in methanol (4 ml) and water (1.3 ml), was added LiOH (17.20 mg, 0.718 mmol) and the mixture was refluxed for 4 hours. The solvent was concentrated at reduced pressure, the residue was dissolved in water and neutralized with HCl (1M). This mixture was purified by C18 column (10 g) to give the title compound (51 mg) as a white solid.

MS; m/z (ES): 390.2 [M+H]⁺

¹H NMR (400 MHz, METHANOL-d₄) d ppm 7.04-7.30 (m, 8H) 4.27 (d, 1H) 4.05 (d, 1H) 3.95-4.07 (m, 1H) 3.55-3.65 (m, 1H) 3.38-3.54 (m, 1H) 3.35 (d, 1H) 3.18 (d, 1H) 2.95-3.20 (m, 2H) 1.85-2.50 (m, 8H) 1.55-1.80 (m, 2H) 1.29 (s, 3H).

### Compound 129: Methyl 1-(11′H-spiro[cyclopentane-1,10′-dibenzo[b,f]oxepin]-3-yl)-3-azetidinecarboxylate

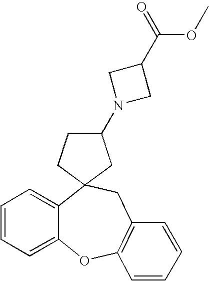

3H,11′H-Spiro[cyclopentane-1,10′-dibenzo[b,f]oxepin]-3-one (Intermediate 20, 70 mg, 0.265 mmol) and methyl 3-azetidinecarboxylate hydrochloride (Intermediate 22, 48.2 mg, 0.318 mmol) in acetonitrile (4 ml) were stirred under nitrogen to give a colorless solution. After stirring for 30 min at room temperature NaBH(OAC)₃ (84 mg, 0.397 mmol) was added and the reaction stirred overnight. Water was added, the solution concentrated under reduced pressure and the aqueous extracted with DCM. The phases were separated on a hydrophobic frit and the combined organic solvent was evaporated. The product was purified using a —NH2 5 g column, eluting with EtOAc/cyclohexane 1:9 to give the title compound (85 mg) as a mixture of two diastereoisomeric racemates.

UPLC/MS Rf=0.65; m/z (ES): 364.06 [M+H]⁺

The diastereomeric mixture was submitted for chiral HPLC purification (Preparative chromatographic conditions: Column=Chiralcel OJ-H; Mobile phase=n-Hexane/Ethanol 95/5% v/v; Flow rate=13 mL/min; DAD=215 nm) to give 1 single isomer and a mixture of other two:

### Compound 130: Methyl 1-(11′H-spiro[cyclopentane-1,10′-dibenzo[b,f]oxepin]-3-yl)-3-azetidinecarboxylate (isomer 1)

retention time=12.97 mins (21 mg); ¹H NMR (400 MHz, CHLOROFORM-d) d ppm 7.30-7.0 (m, 8H); 3.73 (s, 3H); 3.59-2.95 (m, 8H); 2.22-1.59 (m, 6H).

The mixture of the two isomers: retention time=14.95 mins (28 mg) was submitted to further chiral HPLC purification (Preparative chromatographic conditions: Column=Chiralcel AD-H (25×2 cm); Mobile phase=n-Hexane/2-Ethanol 90/10% v/v; Flow rate=13 mL/min; DAD=225 nm) to obtain:

### Compound 131: Methyl 1-(11′H-spiro[cyclopentane-1,10′-dibenzo[b,f]oxepin]-3-yl)-3-azetidinecarboxylate (isomer 3)

retention time=7.5 mins (3 mg); m/z (ES): 364.06 [M+H]⁺

### Compound 132: Methyl 1-(11′H-spiro[cyclopentane-1,10′-dibenzo[b,f]oxepin]-3-yl)-3-azetidinecarboxylate (isomer 2)

retention time=8.82 mins (19 mg); ¹H NMR (400 MHz, CHLOROFORM-d) d ppm 7.29-7.00 (m, 8H); 3.73 (s, 3H); 3.58-2.94 (m, 8H); 2.21-1.58 (m, 6H).

### Compound 133: 1-(11′H-spiro[cyclopentane-1,10′-dibenzo[b,f]oxepin]-3-yl)-3-azetidinecarboxylic acid formic acid salt (isomer 1)

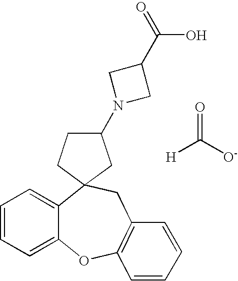

To a colourless solution of methyl 1-(11′H-spiro[cyclopentane-1,10′-dibenzo[b,t]oxepin]-3-yl)-3-azetidinecarboxylate (isomer 1, Compound 130, 21 mg, 0.058 mmol) in methanol (2 ml) and water (1 ml), was added KOH (12.97 mg, 0.231 mmol) and the reaction was stirred at room temperature overnight. MS monitor showed that the reaction was complete. The solvent was removed and the residue taken up with HCl 1M and passed through a HLB 6 g column (water and MeOH to elute) to give the product which was purified by Fraction Lynx acid method to give the title compound as white solid (20 mg);

UPLC/MS Rf=0.57; m/z (ES): 350.04 [M+H]⁺; ¹H NMR (400 MHz, CHLOROFORM-d) d pm 8.36 (bs,1H); 7.25-6.93 (m, 8H); 4.37-3.05 (m, 8H); 2.72-2.58 (m, 6H); 2.37-1.90 (m, 6H).

### Compound 134: 1-(11′H-Spiro[cyclopentane-1,10′-dibenzo[b,f]oxepin]-3-yl)-3-azetidinecarboxylic acid formic acid salt (isomer 2)

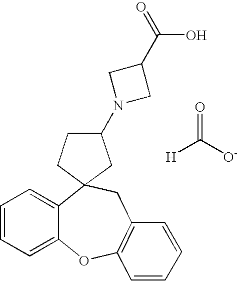

To a colourless solution of methyl 1-(11′H-spiro[cyclopentane-1,10′-dibenzo[b,f]oxepin]-3-yl)-3-azetidinecarboxylate (isomer 2, Compound 132, 19 mg, 0.052 mmol) in methanol (2 ml) and water (1 ml), was added KOH (11.73 mg, 0.209 mmol)and the reaction was stirred at room temperature overnight. UPLC-MS monitor showed that the reaction was complete. The solvent was removed and the product was purified by Fraction Lynx acid method to give the title compound as white solid (20 mg);

UPLC/MS Rf=0.57; m/z (ES): 350.04 [M+H]⁺; ¹H NMR (400 MHz, CHLOROFORM-d) d ppm 8.40 (bs, 1H); 7.22-6.90 (m, 8H); 4.34-2.95 (m, 8H); 2.75-1.84 (m, 6H).

### Compound 135: Methyl[1-(5′,11′-dihydrospiro[cyclopentane-1,10′-dibenzo[a,d]cyclohepten]-3-yl)-3-azetidinyl]acetate

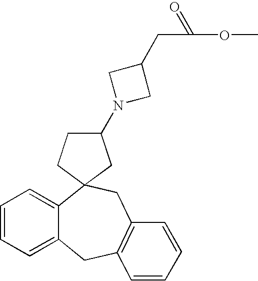

5′,11′-Dihydro-3H-spiro[cyclopentane-1,10′-dibenzo[a,d]cyclohepten]-3-one (Intermediate 5, 110 mg, 0.419 mmol) and methyl 3-azetidinylacetate (Biochemistry, 2006, 45(19) pp 5964-5973, 130 mg, 1.007 mmol) in dry acetonitrile (4 ml) was stirred under nitrogen to give a colourless solution. After stirring for 30 min at room temperature NaBH(OAC)₃ (133 mg, 0.629 mmol) was added and the reaction stirred for 3 hrs. UPLC-MS monitor showed that the reaction was complete. Water was added, the solution concentrated under reduced pressure and the aqueous extracted with DCM. The phases were separated on a hydrophobic frit and the combined organic were evaporated. The product was purified by SCX 5 g column (DCM, MeOH and NH3 0.5 M in MeOH to elute) and the solvent removed to give the title compound (60 mg) as a mixture of two diastereoisomeric racemates;

UPLC/MS Rf=0.56; m/z (ES): 376.13 [M+H]⁺

The diastereomeric mixture was submitted for chiral HPLC purification (Preparative chromatographic conditions: Column=Chiralcel OD-H (25×2.0 cm); Mobile phase=n-Hexane/2-Propanol 92/8% v/v; Flow rate=14 mL/min; DAD=225 nm) to give 2 single isomers:

### Compound 136: Methyl[1-(5′,11′-dihydrospiro[cyclopentane-1,10′-dibenzo[a,d]cyclohepten]-3-yl)-3-azetidinyl]acetate (isomer 1)

retention time=9.73 mins (22 mg); ¹H NMR (400 MHz, CHLOROFORM-d) d ppm 7.31-6.95 (m, 8H); 4.26-4.00 (dd, 2H); 3.70 (s, 3H); 3.58-3.43 (m, 2H); 3.32-3.15 (m, 2H); 3.12-3.00 (m, 1H); 2.94-2.80 (m, 3H); 2.69-2.59 (m, 2H); 2.12-1.65 (m, 6H).

### Compound 137: Methyl[1-(5′,11′-dihydrospiro[cyclopentane-1,10′-dibenzo[a,d]cyclohepten]-3-yl)-3-azetidinyl]acetate (isomer 2)

retention time=10.71 mins (20 mg); ¹H NMR (400 MHz, CHLOROFORM-d) d ppm 7.31-6.98 (m, 8H); 4.22-4.04 (dd, 2H); 3.70 (s, 3H); 3.58-3.43 (m, 2H); 3.32-3.15 (m, 2H); 3.12-3.00 (m, 1H); 2.94-2.80 (m, 3H); 2.69-2.59 (m, 2H); 2.12-1.65 (m, 6H).

### Compound 138: [1-(5′,11′-Dihydrospiro[cyclopentane-1,10′-dibenzo[a,d]cyclohepten]-3-yl)-3-azetidinyl]acetic acid formic acid salt (isomer 1)

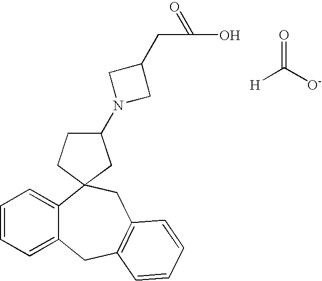

Methyl[1-(5′,11′-dihydrospiro[cyclopentane-1,10′-dibenzo[a,d]cyclohepten]-3-yl)-3-azetidinyl]acetate (isomer 1, Compound 136, 22 mg, 0.059 mmol), KOH (13.15 mg, 0.234 mmol) in water (1 ml) and methanol (4 ml) were stirred at room temperature overnight. MS monitor showed that the reaction was complete. The solvents were removed and the product purified by Fraction Lynx acid method to give the title compound (22 mg);

UPLC/MS Rf=0.63; m/z (ES): 362.11 [M+H]⁺; ¹H NMR (400 MHz, CHLOROFORM-d) d ppm 8.52 (bs, 1H); 7.31-7.22 (m, 1H); 7.21-6.95 (m, 7H); 4.55-3.75 (m, 6H); 3.32-2.64 (m, 6H); 2.54-1.84 (m, 6H).

### Compound 139: [1-(5′,11′-Dihydrospiro[cyclopentane-1,10′-dibenzo[a,d]cyclohepten]-3-yl)-3-azetidinyl]acetic acid formic acid salt (isomer 2)

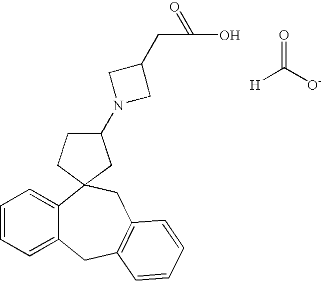

Methyl[1-(5′,11′-dihydrospiro[cyclopentane-1,10′-dibenzo[a,d]cyclohepten]-3-yl)-3-azetidinyl]acetate (isomer 2, Compound 137, 20 mg, 0.053 mmol), KOH (11.95 mg, 0.213 mmol) in water (1 ml) and methanol (4 ml) were stirred at room temperature overnight. MS monitor showed that the reaction was complete. The solvents were removed and the product purified by Fraction Lynx acid method to give the title compound (25 mg).

UPLC/MS Rf=0.63; m/z (ES): 362.11 [M+H]⁺; ¹H NMR (400 MHz, CHLOROFORM-d) d ppm 8.49 (bs, 1H); 7.31-7.22 (m, 1H); 7.21-6.95 (m, 7H); 4.55-3.75 (m, 6H); 3.32-2.64 (m, 6H); 2.54-1.84 (m, 6H).

### Compound 140: Methyl[1-(5′,11′-dihydrospiro[cyclopentane-1,10′-dibenzo[a,d]cyclohepten]-3-yl)-4-piperidinyl]acetate

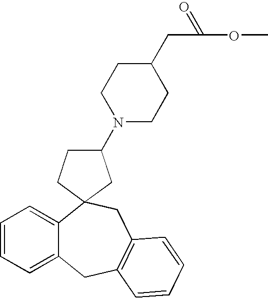

5′,11′-dihydro-3H-spiro[cyclopentane-1,10′-dibenzo[a,d]cyclohepten]-3-one (Intermediate 5, 120 mg, 0.457 mmol) and methyl 4-piperidinylacetate hydrochloride (89 mg, 0.457 mmol) in acetonitrile (4 ml) were stirred under nitrogen at room temperature for 30 mins to give a colourless solution. NaBH(OAC)₃ (194 mg, 0.915 mmol) was added and the reaction stirred for 3 hr. UPLC-MS monitor showed that the reaction was not complete. DIPEA (0.160 ml, 0.915 mmol) and AcOH (0.131 ml, 2.287 mmol) were added and the reaction stirred overnight. UPLC-MS monitor showed that the reaction was not complete so methyl 4-piperidinylacetate hydrochloride 0.5 eq. and after 30 min NaBH(OAC)₃ 70 mg were added and the reaction mixture stirred till the day after. Water was added and the solution was concentrated under reduced pressure then the aqueous extracted with DCM. The phases were separated on a hydrophobic frit and the combined organic solvent was evaporated. The product was purified using a —NH₂ 10 g column (EtOAc/cyclohexane 1:9 to 1:1 to elute), the solvent removed to give the title compound (180 mg) as a mixture of two diastereoisomeric racemates;

UPLC/MS Rf=0.63; m/z (ES): 404.12 [M+H]⁺

The diastereomeric mixture was submitted for chiral HPLC purification (Preparative chromatographic conditions: Column=Chiralcel AD-H (25×0.46 cm); Mobile phase=n-Hexane/Ethanol+0.1% isopropylammina 93/7 v/v; Flow rate=14 mL/min; DAD=220 nm) to give: diastereoisomeric mixture 1 (retention time=6.25 mins, 77 mg) and diastereoisomeric mixture 2 (retention time=7.49 mins, 75 mg).

The two mixtures were submitted to further chiral HPLC purification (Preparative chromatographic conditions: Column=Chiralcel OJ-H; Mobile phase=n-Hexane/2-Ethanol+0.1% isopropylammina 70/30 v/v and 75/25 v/v; Flow rate=13 mL/min; DAD=220 nm) to give:

### Compound 141: Methyl[1-(5′,11′-dihydrospiro[cyclopentane-1,10′-dibenzo[a,d]cyclohepten]-3-yl)-4-piperidinyl]acetate (isomer 1)

retention time=10.18 mins (62 mg); ¹H NMR (400 MHz, CHLOROFORM-d) d ppm 7.35-6.98 (m, 8H); 4.32-3.97 (dd, 2H); 3.71 (s, 3H); 3.33-2.85 (m, 5H); 2.30-1.30 (m, 15H).

### Compound 142: Methyl[1-(5′,11′-dihydrospiro[cyclopentane-1,10′-dibenzo[a,d]cyclohepten]-3-yl)-4-piperidinyl]acetate (isomer 3)

retention time=8.64 mins (6.6 mg); UPLC/MS Rf=0.65; m/z (ES): 404.19 [M+H]⁺

### Compound 143: Methyl[1-(5′,11′-dihydrospiro[cyclopentane-1,10′-dibenzo[a,d]cyclohepten]-3-yl)-4-piperidinyl]acetate (isomer 2)

retention time=11.74 mins (58 mg); ¹H NMR (400 MHz, CHLOROFORM-d) d ppm 7.35-6.98 (m, 8H); 4.32-3.97 (dd, 2H); 3.71 (s, 3H); 3.33-2.85 (m, 5H); 2.30-1.30 (m, 15H).

### Compound 144: Methyl[1-(5′,11′-dihydrospiro[cyclopentane-1,10′-dibenzo[a,d]cyclohepten]-3-yl)-4-piperidinyl]acetate (isomer 4)

retention time=13.16 mins (4.5 mg); UPLC/MS Rf=0.65; m/z (ES): 404.19 [M+H]⁺

### Compound 145: [1-(5′,11′-Dihydrospiro[cyclopentane-1,10′-dibenzo[a,d]cyclohepten]-3-yl)-4-piperidinyl]acetic acid (isomer 1)

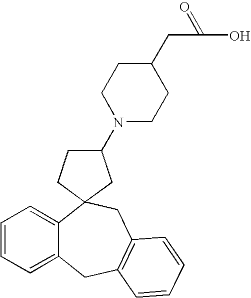

Methyl[1-(5′,11′-dihydrospiro[cyclopentane-1,10′-dibenzo[a,d]cyclohepten]-3-yl)-4-piperidinyl]acetate (isomer 1, Compound 141, 62 mg, 0.154 mmol) and KOH (34.5 mg, 0.615 mmol) in methanol (3 ml) and water (1 ml) were stirred overnight. UPLC-MS monitor showed the reaction was complete. The solvent was removed and the residue taken up with HCl 1M and passed through a HLB 6 g column (water and MeOH to elute) to give the title compound (60 mg);

UPLC/MS Rf=0.58; m/z (ES): 390.09 [M+H]⁺; ¹H NMR (400 MHz, CHLOROFORM-d) d ppm 7.55-7.50 (m, 1H); 7.24-6.98 (m, 7H); 4.15-4.05 (dd, 2H); 3.86-3.25 (m, 5H); 2.92-1.73 (m, 15H).

### Compound 146: [1-(5′,11′-Dihydrospiro[cyclopentane-1,10′-dibenzo[a,d]cyclohepten]-3-yl)-4-piperidinyl]acetic acid (isomer 2)

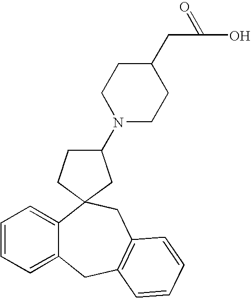

Methyl[1-(5′,11′-dihydrospiro[cyclopentane-1,10′-dibenzo[a,d]cyclohepten]-3-yl)-4-piperidinyl]acetate (isomer 2, Compound 143, 58 mg, 0.144 mmol) and KOH (32.3 mg, 0.575 mmol) in methanol (3 ml) and water (1 ml) were stirred overnight. UPLC-MS monitor showed the reaction was complete. The solvent was removed and the residue taken up with HCl 1M and passed through a HLB 6 g column (water and MeOH to elute) to give the title compound (55 mg);

UPLC/MS Rf=0.58; m/z (ES): 390.12 [M+H]⁺; ¹H NMR (400 MHz, CHLOROFORM-d) d ppm 7.59-7.47 (m, 1H); 7.24-6.96 (m, 7H); 4.21-4.01 (dd, 2H); 3.91-3.24 (m, 5H); 2.85-1.81 (m, 15H).

### Compound 147: Methyl 1-(5′,11′-dihydrospiro[cyclopentane-1,10′-dibenzo[a,d]cyclohepten]-3-yl)-3-fluoro-3-piperidinecarboxylate (diastereomeric mixture 2)

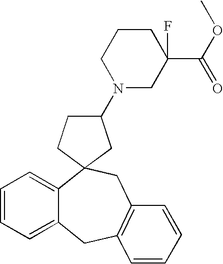

To a solution of (+)5′,11′-dihydro-3H-spiro[cyclopentane-1,10′-dibenzo[a,d]cyclohepten]-3-one (Intermediate 7, 22.5 mg, 0.09 mmol) in methanol (2 ml), ethyl 3-fluoro-3-piperidinecarboxylate (enantiomer 1, Intermediate 27A, 15 mg, 0.09 mmol) and zinc chloride (5.83 mg, 0.04 mmol) were added to give suspension. The reaction mixture was stirred for 1.5 hour and then sodium cyanoborohydride (21.5 mg, 0.34 mmol) was added. After 84 hours solvent was evaporated and the crude product was purified by SCX to give a mixture of methyl ester and the corresponding acid (26 mg) (transesterification and partial hydrolysis occurred in the reaction). This mixture was dissolved in DCM (2 ml) and MeOH (0.5 ml) and then trimethylsilyldiazomethane (0.15 ml) was added and the reaction mixture was stirred at room temperature for 16 hours. After evaporation of volatiles the crude product was purified by flash chromatography on silica gel (5 g) eluting with a gradient of MeOH in DCM (from 1 to 3%) to give the title compound (22 mg) as mixture of two diastereoisomers.

The diastereomeric mixture was submitted for chiral HPLC purification (Preparative chromatographic conditions: Column=Chiralcel OD-H (25×0.46 cm); Mobile phase=n-Hexane/Ethanol 70/30 v/v; Flow rate=1 mL/min; DAD=225 nm) to give:

### Compound 148: Methyl 1-(5′,11′-dihydrospiro[cyclopentane-1,10′-dibenzo[a,d]cyclohepten]-3-yl)-3-fluoro-3-piperidinecarboxylate (isomer 2)

retention time=5.7 mins (13.2 mg); m/z (ES): 408.16 [M+H]⁺;

1H NMR (400 MHz, CHLOROFORM-d) d ppm 6.99-7.30 (m, 8H) 4.25 (d, 1H) 4.03 (d, 1H) 3.81 (s, 3H) 3.27 (d, 1H) 3.13 (d, 1H) 3.06 (m, 3H) 2.51-2.67 (m, 1H) 1.57-2.26 (m, 11H)

### Compound 149: Methyl 1-(5′,11′-dihydrospiro[cyclopentane-1,10′-dibenzo[a,d]cyclohepten]-3-yl)-3-fluoro-3-piperidinecarboxylate (isomer 4)

retention time=6.9 mins (3.2 mg); m/z (ES): 408.20 [M+H]⁺;

### Compound 150: 1-(5′,11′-Dihydrospiro[cyclopentane-1,10′-dibenzo[a,d]cyclohepten]-3-yl)-3-fluoro-3-piperidinecarboxylic acid (isomer 2)

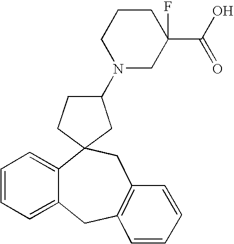

Methyl 1-(5′,11′-dihydrospiro[cyclopentane-1,10′-dibenzo[a,d]cyclohepten]-3-yl)-3-fluoro-3-piperidinecarboxylate (isomer 2, Compound 148, 13.2 mg, 0.032 mmol) was dissolved in Tetrahydrofuran (1 ml) and Water (0.5 ml). LiOH (3.88 mg, 0.162 mmol) was added and the mixture was stirred at reflux for 3 hours. Solvents were evaporated, water (2 ml) was added. pH of suspension was adjusted to 6-7 using 1N HCl solution. White emulsion was obtained. This emulsion was purified on C 18 column to give, after trituration with diethyl ether, the title compound (6 mg) as white solid.

m/z (ES): 394.0 [M+H]⁺; 1H NMR (400 MHz, CHLOROFORM-d) d ppm 7.04-7.35 (m, 8H) 4.04-4.29 (m, 2H) 3.53-3.67 (m, 1H) 3.37-3.51 (m, 1H) 3.05-3.29 (m, 3H) 1.29-2.66 (m, 12H)

Biological Assay

a) H₁ Antagonist Assay

Adherent Chinese Hamster Ovary (CHO) cells stably expressing the recombinant human H₁ receptor were maintained in culture at 37° C. under 5% CO₂ in Alpha Minimum Essential Medium without ribonucleosides (Gibco Invitrogen), supplemented with 10% dialysed foetal calf serum and 200 mM Glutamine. These cells, expressing the human H₁ receptor, were snap frozen and stored ready for assay. 24 or 72 hours prior to assay the cells were seeded into black-walled, clear-based 384-well plates at a density of 12,000 or 4 000 cells per well (respectively) and cultured at 37° C. under 5% CO₂. Cell seeding densities resulted in a confluent monolayer of cells at a time point of approximately 24 hours for 1200 cells or 72 hours for 4 000 cells. The media was removed by aspiration and the cells were then incubated with HBSS medium (CaCl₂.2H₂O 1.26 Mm, Glucose 5.55 mM, KCl 5.36 mM MgSO₄(anhyd) 0.81 mM, NaCl 136.89 mM, KH₂PO₄(anhyd) 0.41 mM, HEPES 20 mM, NaHCO₃ 4.16 mM) containing the cytoplasmic calcium indicator, Fluo-4 in the acetylmethyl form (4 mM), 2.5 mM Probenecid and 250 uM Brilliant Black (Molecular Devices) at 37° C. for 60 min. The loaded cells were then incubated with test compound for 30 min at 37° C. The plates were then placed into a FLIPR (Molecular Devices, UK) for testing in antagonist mode, where a pre-determined concentration of histamine (approximately 4×EC50) was added while cell fluorescence (λex 488 nm, λem 540 nm) was monitored.

Supporting compounds 1-20, 31, 32, 53, 54, 58, 59, 63, 64, 68, 69, 75, 76, 80, 81, 87-89, 93, 94, 99, 100, 102, 104, 106, 108, 114-116, 120-123, 127, 128, 133, 134, 138, 139-145, 146 and 150 gave an fpki against H₁ in the range 6.0-9.2.

b) 5HT_{2A} Antagonist Assay

Adherent SH-SY5Y cells stably expressing recombinant human 5-HT_{2A} were maintained in culture at 37° C. under 5% CO₂ in Alpha Minimum Essential Medium +ribonucleosides (Gibco Invitrogen,) supplemented with 10% dialysed foetal calf serum and 400 micrograms geneticin. SH-SY5Y cells are neuroblastoma and are commercially available from the American Type Culture Collection (ATCC). The SH-SY5Y cells, expressing 5-HT_{2A} receptors, were seeded into black-walled clear-based 384-well plates at a density of 16,000 cells per well and cultured overnight at 37° C. under 5% CO₂. The media was removed by aspiration and the cells were then incubated with HBSS medium (CaCl₂.2H₂O 1.26 Mm, Glucose 5.55 mM, KCl 5.36 mM MgSO₄(anhyd) 0.81 mM, NaCl 136.89 mM, KH₂PO₄(anhyd) 0.41 mM, HEPES 20 mM, NaHCO₃ 4.16 mM) containing cytoplasmic calcium indicator, Fluo-4 in the acetylmethyl form (4 mM), 2.5 mM Probenecid and 250 μM Brilliant Black (Molecular Devices) at 37° C. for 60 min. The loaded cells were then incubated with test compound for 30 min at 37° C. The plates were then placed into a FLIPR (Molecular Devices, UK) for testing in antagonist mode, where a pre-determined concentration of 5-HT (approximately 4×EC50) was added while cell fluorescence (λex 488 nm, λem 540 nm) was monitored.

Supporting compounds 1-7, 9-18, 20, 31, 32, 59, 69, 75, 88, 94, 100, 102, 106, 108, 122 and 123 gave an fpki against 5HT_{2A} in the range 5.8-8.8.

Alternatively, some of the supporting compounds were tested in the following 5HT_{2A} antagonist assay.

Frozen Human Embryonic Kidney (HEK) cells stably expressing the human 5-HT_{2A} serotonin receptor and aequorin apo-protein were thawed and added drop wise to an appropriate volume of warm DMEM media (Gibco Invitrogen 41965-039) containing 10% dialysed foetal bovine serum (FBS) (Invitrogen; 05-4011DK). Cells were then spun down at 1000 rpm for 5 minutes at room temperature. The supernatant was poured off and the pellet re-suspended in HBSS buffer (Sigma kit H1387) supplemented with HEPES (Sigma H0887), NaHCO₃ (Sigma S8761)) containing 0.1% Pluronic Acid F68 solution (Gibco Invitrogen; 24040-032) and 0.1% Bovine Serum Albumin (CalBiochem; 126609)). A sample was taken and a cell count performed. Cells were diluted down to 2.5e6 cells/mi in loading buffer, Coelenterazine [5 uM] (Invitrogen; C6780) was added to the cell suspension and cell vessel wrapped in foil. Cell vessel was put on windmill rotator (Bibby Stuart) and left overnight at room temperature. Before assay, a sample was taken and a cell count performed. Cells were diluted to an appropriate final density immediately prior to assay. Plates containing compounds (0.5 ul) were placed in a Lumilux, where they were diluted in buffer (20 ul), before additions of cells (20 ul) and a pre-determined sub-maximal concentration of 5-HT (20 ul) whilst luminescence was monitored. Data was analysed using area under curve for the entire timecourse, normalised to in-plate nominal high and low controls and fitted to a four parameter logistic equation.

Supporting compounds 1, 3-5, 7, 10-15, 18, 20, 53, 54, 58, 59, 64, 69, 75, 76, 80, 81, 87-89, 93, 94, 100, 102, 106, 108, 115, 116, 121-123, 127, 128, 133, 134, 138, 139, 146 and 150 gave an fpki against 5HT_{2A} in the range 5.6-9.0.

1. A compound of formula (I) or a pharmaceutically acceptable salt thereof: 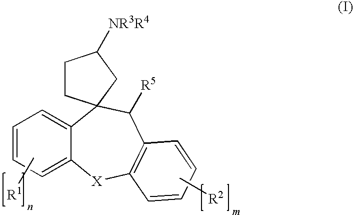
wherein:
X is CH₂, C═O, O, or S;
n is 0, 1 or 2;
m is 0, 1 or 2;
when present, R¹ is independently selected from the list consisting of C₁₋₄alkyl, C₁₋₄alkoxy and halogen;
when present, R² is independently selected from the list consisting of C₁₋₄alkyl, C₁₋₄alkoxy and halogen;
R³ and R⁴ are independently selected from the list consisting of hydrogen, C₁₋₆alkyl, and carboxyC₁₋₆alkyl; or
R³ and R⁴, together with the nitrogen to which they are attached, form a 4-7 membered saturated or partially unsaturated ring optionally containing one or more additional heteroatoms independently selected from N, S and O, the ring being optionally substituted by one or more groups independently selected from halogen, C₁₋₃alkoxycarbonyl, carboxy, carboxyC₁₋₆alkyl, hydroxy and —C(O)NR^{a}R^{b}; or
R³ and R⁴, together with the nitrogen to which they are attached, form a 6 membered azabicyclic ring optionally substituted by one or more groups independently selected from halogen, C₁₋₃alkoxycarbonyl, carboxy, C₁₋₆alkyl, carboxyC₁₋₆alkyl, hydroxy and —C(O)NR^{a}R^{b};
R^{a} and R^{b} are independently selected from the list consisting of hydrogen and C₁₋₃alkyl; and
R⁵ is hydrogen or oxo.

2. The compound according to claim 1 or a pharmaceutically acceptable salt thereof, wherein X is CH₂ or O.

3. The compound according to claim 1 or a pharmaceutically acceptable salt thereof, wherein R³ and R⁴, together with the nitrogen to which they are attached, form a 5-6 membered saturated or partially unsaturated ring optionally containing one additional heteroatom selected from N, S and O, the ring being optionally substituted by one or more groups selected independently from halogen, C₁₋₃alkoxycarbonyl, carboxy and C₁₋₆alkyl.

4. The compound according to claim 1 or a pharmaceutically acceptable salt thereof, wherein R³ and R⁴, together with the nitrogen to which they are attached, form 6 membered saturated or partially unsaturated ring, the ring being optionally substituted by one or more groups selected independently from halogen, C₁₋₃alkoxycarbonyl, carboxy and C₁₋₆alkyl.

5. The compound according to claim 1 or a pharmaceutically acceptable salt thereof, wherein R³ and R⁴, together with the nitrogen to which they are attached, form a 6 membered azabicyclic ring optionally substituted by one or more groups independently selected from halogen, carboxy and C₁₋₆alkyl.

6. The compound according to claim 1 or a pharmaceutically acceptable salt thereof, wherein R³ and R⁴, together with the nitrogen to which they are attached, form an azetidinyl, the ring being optionally substituted by one or more groups selected independently from halogen, C₁₋₃alkoxycarbonyl, carboxy and C₁₋₆alkyl.

7. The compound according to claim 1 wherein, the compound of formula (I) is selected from the list consisting of:
(−)1-(5′,11′-Dihydrospiro[cyclopentane-1,10′-dibenzo[a,d]cyclohepten]-3-yl)-4-piperidinecarboxylic acid;
3-(5′,11′-Dihydrospiro[cyclopentane-1,10′-dibenzo[a,d]cyclohepten]-3-yl)-3-azabicyclo[3.1.0]hexane-1-carboxylic acid, isomer 2;
1-(5′,11′-Dihydrospiro[cyclopentane-1,10′-dibenzo[a,d]cyclohepten]-3-yl)-3-azetidinecarboxylic acid formate salt, isomer 1;
(−)1-(5′,11′-Dihydrospiro[cyclopentane-1,10′-dibenzo[a,d]cyclohepten]-3-yl)-3-azetidinecarboxylic acid hydrochloride salt;
(−)1-(5′,11′-Dihydrospiro[cyclopentane-1,10′-dibenzo[a,d]cyclohepten]-3-yl)-3-azetidinecarboxylic acid; and
1-(11′H-Spiro[cyclopentane-1,10′-dibenzo[b,f]oxepin]-3-yl)-3-azetidinecarboxylic acid, isomer;
or a pharmaceutically acceptable salt thereof.

8. 1-(5′,11′-Dihydrospiro[cyclopentane-1,10′-dibenzo[a,d]cyclohepten]-3-yl)-3-azetidinecarboxylic acid or a pharmaceutically acceptable salt thereof.

9. (−)1-(5′,11′-Dihydrospiro[cyclopentane-1,10′-dibenzo[a,d]cyclohepten]-3-yl)-3-azetidinecarboxylic acid or a pharmaceutically acceptable salt thereof.

10-12. (canceled)

13. A method of treatment of a disease or condition mediated by antagonism of the H₁ receptor in a human, which comprises administering to said human a therapeutically effective amount of the compound or a pharmaceutically acceptable salt thereof, as claimed in claim 1.

14. The method as claimed in claim 13, wherein the disease or condition is a sleep disorder.

15-16. (canceled)

17. A pharmaceutical composition comprising the compound as defined in claim 1 or a pharmaceutically acceptable salt thereof, and a pharmaceutically acceptable carrier or excipient.

18. A process for preparing the pharmaceutical composition as defined in claim 17, the process comprising mixing the compound as defined in claim 1 or a pharmaceutically acceptable salt thereof and a pharmaceutically acceptable carrier or excipient.
